(19) United States
(12) Patent Application Publication
(10) Pub. No.: US 2015/0343049 A1
(43) Pub. Date: Dec. 3, 2015

(54) Vaccine

(71) Applicant: Simon Michael Cutting, Egham-Surrey (GB)

(72) Inventor: Simon Michael Cutting, Egham-Surrey (GB)

(73) Assignee: Royal Holloway and Bedford New College, University of London, Surrey (GB)

(21) Appl. No.: 14/685,746

(22) Filed: Apr. 14, 2015

(30) Foreign Application Priority Data
Apr. 14, 2014 (GB) 1406628.6

(51) Int. Cl. A61K 39/08; G01N 33/569

(52) U.S. Cl. CPC A61K 39/08; G01N 33/56911; G01N 2333/33; G01N 2469/10; A61K 2039/58

(57) ABSTRACT

The invention relates to vaccines, and particularly to vaccines active against pathogenic bacteria including Clostridia species, such as C. difficile, and Bacillus species, such as B. anthracis and B. cereus. The invention is concerned with the use of nucleic acids and proteins as antigens for use in vaccine design and construction, and to the vaccines per se. The nucleic acids and proteins are also useful in diagnostic test kits and methods for the detection of Clostridium spp. and Bacillus spp. infections.

The present invention relates to vaccines, and particularly to vaccines active against pathogenic bacteria including Clostridia species, such as C. difficile, and Bacillus species, such as B. anthracis and B. cereus. The invention is particularly concerned with the use of nucleic acids and proteins as antigens for use in vaccine design and construction, and to the vaccines per se. The nucleic acids and proteins are also useful in diagnostic test kits and methods for the detection of Clostridium spp. and Bacillus spp. infections.

Clostridium difficile is a leading cause of nosocomial antibiotic-associated diarrhea in industrialized countries (Rupnik et al., 2009). This spore forming bacterium is able to colonize the gastro-intestinal (GI) tract of infected patients and, during antibiotic therapy, the resulting disturbance to the natural gut microflora promotes germination of C. difficile spores, outgrowth and proliferation of live cells (Songer & Anderson, 2006) followed by shedding of large numbers of spores in the feces (Lawley et al., 2009a). Disease is caused mainly by the production of two toxins, A (TcdA) and B (TcdB), which leads to diarrhoea and in more severe cases, pseudomembrane colitis (Rupnik et al., 2009). The spore of C. difficile is the dormant state of this organism and the primary agent of transmission (Gerding et al., 2008). This has been supported by recent studies where a mutant strain of C. difficile, unable to produce the Sp000A protein (a transcriptional regulatory protein essential for the initiation of sporulation) fails to persist and transmit the disease (Deakin et al., 2012). Interestingly, mice infected with C. difficile can exist in two physiological states, a carrier state, where low levels of C. difficile spores are shed in the feces and a ‘supershedder’ state where large numbers of spores are shed (Lawley et al., 2009a). This ‘supershedder’ state is induced following antibiotic treatment and most closely resembles the clinical situation where patients contract C. difficile infection. Capable of withstanding heat, desiccation and noxious chemicals, spores transmit C. difficile outside of the host and therefore present a major burden to hospitals in containment and disinfection (Gerding et al., 2008).

Strains producing neither toxin are completely attenuated in the hamster model of infection (Kuehne et al., 2010) yet non-toxigenic strains have been found to be endowed with vaccine-strain attributes. Although toxin A and toxin B are considered the two main virulence factors, others cannot be excluded; for example, it has been shown that hamsters challenged with spores of the non-toxigenic strain CD1342 showed mild caecal pathology characterized by local acute epithelial cell loss, hemorrhagic congestion and neutrophil infiltration (Buckley et al., 2013). Hamsters colonized with non-toxigenic strains, M₃ and T₇, were protected against challenge with toxigenic B1 group strains (Nagaro et al., 2013), suggesting non-toxic strains can exclude toxigenic strains from colonization. However, the mechanism for how these non-toxigenic strains confer protection remains both intriguing and unclear.

The role of the spore in transmission of the disease suggests that this dormant life form may play a key role in colonization, a process better divided into three stages: establishment of infection, maintenance of infection (persistence) and spore shedding. Spores of C. difficile resemble those of other Gram-positive spore-formers but differ somewhat in the abundance of enzymes they carry on their surface layers including three catalases and a bifunctional peroxiredoxin-chitinase (Permpoonpattana et al., 2011b, Permpoonpattana et al., 2013). C. difficile spores also carry a poorly defined outer surface layer whose function has been linked to germination, adhesion and resistance properties of the spore (Henriques & Moran, 2007, Lawley et al., 2009b, Escobar-Cortes et al., 2013). This outermost layer of C. difficile spores has some similarities to the exosporium of some spore formers but conflicting published data has delayed a definitive assignment. The BclA (bacillus collagen-like protein of anthracis) glycoprotein is a major component of the exosporium in some spore formers that can form hair-like filaments and carries collagen-like repeats of the amino-acid triplet GPT used for attachment to oligosaccharides (Steichen et al., 2003, Sylvestre et al., 2002, Sylvestre et al., 2003). A second collagen-like protein, BclB has also been identified in B. anthracis and has been linked to exosporium assembly (Thompson & Stewart, 2008, Waller et al., 2005).

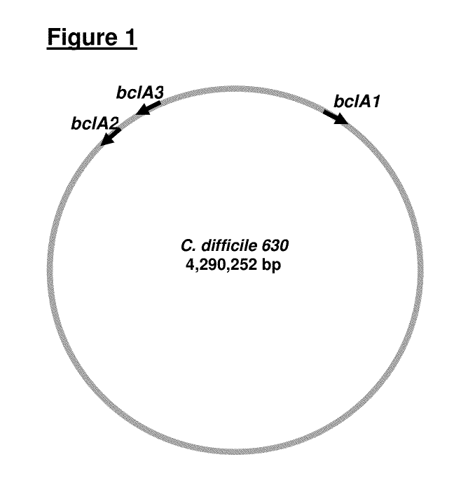

The BclA protein is a major component of the outermost layer of spores of a number of bacterial species and Clostridium difficile carries three bclA genes (see FIG. 1). As described in the Examples, using insertional mutagenesis, the inventors have characterized each bclA gene, and found that spores devoid of these BclA proteins had surface aberrations, reduced hydrophobicity and germinated faster than wild-type spores. Analysis of infection and colonization in mice and hamsters revealed that the 50% infectious dose (ID₅₀) of spores was surprisingly higher (i.e., 2 logs) in the bclA1⁻ mutant compared to the isogenic wild-type control, but that levels of toxins (A and B) were indistinguishable from animals dosed with wild-type spores. Moreover, bclA1⁻ spores germinated surprisingly faster than wild-type spores, yet mice were less susceptible to infection suggesting that BclA1 must play a key role in the initial (i.e., pre-spore germination) stages of infection. The inventors have therefore convincingly established that the BclA protein can be used as an effective antigen in vaccine development to create a novel vaccine.

Thus, in a first aspect of the invention, there is provided a vaccine comprising a C. difficile BclA polypeptide, or a fragment or variant thereof.

According to a second aspect of the invention, there is provided the use of a C. difficile BclA polypeptide, or a fragment or variant thereof, for the development of a vaccine.

The genome of C. difficile strain 630 has three genes encoding BclA-like proteins, annotated as bclA1, bclA2 and bclA3, which encode proteins with predicted masses of 67.8, 49.0 and 58.2 kDa, respectively. It will be appreciated that the term “BclA” refers to “bacillus collagen-like protein of anthracis”. However, other species, such as B. cereus also comprise functional homologues, and embodiments of this invention refer to the C. difficile homologues of BclA proteins and bclA genes.

For example, the DNA sequence of C. difficile bclA1 (Locus tag=CD0332 as described in Sebaihia, M at al. (2006) Nat Genet 38: 779-786. [bclA1 C. difficile 630 nt gi|126697566:399494-402199]) is provided herein as SEQ ID No:1, as follows:

[SEQ ID No: 1]

ATGAGAAATATTATACTTTATTTAAATGATGATACTTTTATATCTAAAAA
ATATCCAGATAAAAACTTTAGTAATTTAGATTATTGCTTAATAGGAAGTA
AATGTTCAAATAGTTTTGTAAAAGAAAAGTTGATTACTTTTTTTAAAGTG
AGAATACCAGATATATTAAAAGACAAAAGTATATTAAAAGCAGAGTTATT
TATTCATATTGATTCAAATAAGAATCATATTTTTAAAGAAAAAGTAGATA
TTGAAATTAAAAGAATAAGTGAATATTATAATTTACGAACTATAACATGG
AATGATAGAGTGTCTATGGAAAATATCAGGGGATATTTACCAATTGGGAT
AAGTGATACATCCAACTATATTTGTTTAAATATTACGGGAACTATAAAAG
CATGGGCAATGAATAAATATCCTAATTATGGGTTAGCTTTATCTTTAAAT
TACCCTTATCAGATTCTTGAATTTACATCTAGTAGAGGTTGTAACAAACC
GTATATACTTGTAACATTTGAAGATAGAATTATAGATAATTGTTATCCTA
AATGTGAGTGTCCTCCAATTAGAATTACAGGTCCAATGGGACCAAGAGGA
GCGACAGGAAGTACAGGACCAATGGGAGTAACAGGCCCAACCGGAAGTAC
AGGAGCGACAGGAAGCATAGGACCAACAGGCCCAACCGGAAATACAGGAG
CAACAGGAAGTATAGGGCCAACGGGAGTAACAGGCCCAACCGGAAGTACA
GGAGCGACAGGAAGTATAGGACCAACAGGAGTAACAGGTCCGACAGGAAA
TACGGGAGTGACAGGAAGTATAGGACCAACGGGAGCAACAGGCCCGACAG
GAAATACGGGAGTGACAGGAAGTATAGGACCAACAGGAGTAACAGGCCCA
ACAGGAAATACAGGAGAAATAGGACCAACGGGAGCAACAGGTCCAACAGG
AGTGACAGGAAGTATAGGACCAACAGGAGCAACAGGACCAACAGGAGAAA
TAGGACCAACGGGAGCAACAGGAGCGACAGGAAGTATAGGACCAACAGGA
GCAACAGGTCCAACAGGAGCGACAGGAGTGACAGGAGAAATAGGGCCAAC
AGGAGAAATAGGACCAACGGGAGCAACAGGCCCAACAGGAGTGACAGGAA
GTATAGGACCAACGGGAGCAACAGGCCCAACAGGAGCGACAGGAGAAATA
GGACCAACAGGAGCAACAGGCCCAACAGGAGTGACAGGAAGTATAGGACC
AACGGGAGCAACAGGCCCAACAGGAGCGACAGGAGAAATAGGACCAACGG
GAGCAACAGGCCCAACAGGAGTAACAGGAGAAATAGGACCAACGGGAGCA
ACAGGCCCAACAGGAAATACAGGAGTAACAGGAGAAATAGGACCAACGGG
AGCAACGGGTCCGACAGGAAATACAGGAGTGACAGGAGAAATAGGACCAA
CGGGAGCAACAGGACCAACAGGAGTGACAGGAGAAATAGGGCCAACAGGA
AATACAGGAGCGACAGGAAGTATAGGGCCAACGGGAGTAACAGGTCCAAC
AGGAGCGACAGGAAGTATAGGACCAACGGGAGCAACAGGAGCGACAGGAG
TAACAGGACCAACAGGTCCAACAGGAGCAACAGGCAATTCCTCTCAGCCA
GTTGCTAACTTCCTCGTAAATGCACCATCTCCACAAACACTAAATAATGG
AGATGCTATAACAGGTTGGCAAACAATAATAGGAAATAGTTCAAGTATAA
CAGTAGATACAAATGGTACGTTTACAGTACAAGAAAATGGTGTGTATTAT
ATATCAGTTTCAGTAGCATTACAACCAGGTTCATCAAGTATAAATCAATA
TTCTTTCGCTATCCTATTCCCAATTTTAGGAGGAAAAGATTTGGCAGGGC
TTACTACTGAGCCAGGAGGCGGAGGAGTACTTTCTGGATATTTTGCTGGT
TTTTTATTTGGTGGGACTACTTTTACAATAAATAATTTTTCATCTACAAC
AGTAGGGATACGAAATGGGCAATCAGCAGGAACTGCGGCTACTTTGACGA
TATTTAGAATAGCTGATACTGTTATGACTTAA

The DNA sequence of C. difficile bclA2 (Locus tag=CD3230 as described in Sebaihia, M at al. (2006) Nat Genet 38: 779-786 [lcl∥CD3230|bclA2|74723015 exosporium glycoprotein]) is provided herein as SEQ ID No:2, as follows:

[SEQ ID No: 2]

ATGAGTGATATTTCAGGTCCAAGTTTATATCAAGATGTAGGTCCAACAGG
GCCAACAGGTGCTACTGGTCCAACAGGACCGACGGGGCCTAGAGGTGCAA
CTGGAGCGACCGGAGCAAATGGAATAACAGGACCAACAGGAAATACAGGA
GCAACTGGAGCGAATGGAATAACGGGTCCAACAGGAAATATGGGAGCGAC
TGGACCAAATGGAACAACAGGTTCTACAGGACCAACAGGAAATACAGGAG
CGACTGGAGCGAATGGAATAACAGGTCCGACAGGGAATACAGGAGCAACC
GGAGCAAATGGAATAACAGGACCAACAGGAAACAAAGGAGCAACCGGAGC
AAATGGAATAACAGGTTCTACAGGACCAACAGGAAATACAGGAGCGACTG
GAGCAAATGGAATAACAGGTCCAACAGGGAATACAGGAGCAACAGGAGCA
ACAGGTCCAACCGGACTAACAGGAGCAACAGGAGCAACCGGAGCAAATGG
AATAACAGGACCAACAGGAAATACAGGAGCAACCGGAGCAAATGGAGTAA
CAGGTGCTACAGGCCCAACAGGAAATACAGGAGCAACAGGTCCAACAGGA
AGTATAGGAGCGACTGGAGCAACAGGAACAACTGGGGCAACAGGCCCAAT
AGGAGCAACAGGAGCAACAGGAGCAGATGGAGAGGTAGGTCCAACAGGAG
CAGTAGGAGCAACAGGTCCAGATGGTTTGGTAGGTCCAACAGGCCCAACA
GGCCCAACCGGAGCAACCGGAGCAAATGGTTTGGTAGGCCCAACAGGCCC
AACCGGAGCAACCGGAGCAAATGGTTTGGTAGGTCCAACAGGAGCGACAG
GAGCAACAGGAGTAGCTGGGGCAATAGGTCCAACGGGAGCAGTAGGAGCA
ACAGGCCCAACGGGAGCAGATGGAGCAGTAGGTCCAACAGGAGCGACAGG
AGCAACAGGGGCAAATGGAGCAACAGGCCCAACGGGAGCAGTAGGAGCAA
CGGGAGCGAATGGAGTAGCAGGTCCAATAGGTCCAACAGGTCCAACGGGA
GAAAATGGAGTAGCAGGAGCAACAGGAGCGACAGGAGCAACAGGGGCAAA
TGGAGCAACAGGCCCAACAGGAGCAGTAGGAGCAACGGGAGCAAATGGAG
TAGCAGGAGCGATAGGACCAACAGGCCCAACCGGAGCAAATGGAGCGACA
GGAGCAACAGGGGCGACAGGAGCAACAGGAGCAAATGGAGCAACAGGTCC
AACCGGAGCGACAGGAGCAACAGGAGTGTTAGCAGCAAACAATGCACAAT
TTACAGTGTCCTCTTCAAGTTTAGTGAATAATACATTAGTGACATTTAAT
TCATCATTTATAAATGGAACTAATATAACTTTTCCAACAAGTAGTACTAT
AAATCTTGCAGTTGGAGGGATATACAATGTATCTTTCGGTATACGTGCCA
CACTTTCACTTGCAGGATTTATGTCAATTACTACTAACTTTAATGGAGTA
ACTCAAAATAACTTTATTGCAAAAGCAGTAAATACACTTACTTCATCAGA
TGTAAGTGTAAGTTTAAGCTTTTTAGTTGATGCTAGAGCAGCAGCTGTTA
CTTTAAGTTTTACATTTGGTTCAGGCACGACAGGTACTTCTGCAGCTGGA
TATGTATCAGTTTATAGAATACAATAG

The DNA sequence of C. difficile bclA3 (Locus tag=CD3349 as described in Sebaihia, M at al. (2006) Nat Genet 38: 779-786 [lcl∥CD3349|bclA3|74729191 putative exosporium glycoprotein]) is provided herein as SEQ ID No:3, as follows:

[SEQ ID No: 3]

GTGCTTTTAATAATGAGTAGAAATAAATATTTTGGACCATTTGATGATAA
TGATTACAACAATGGCTATGATAAATATGATGATTGTAACAATGGTCGTG
ATGATTATAATAGCTGTGATTGCCATCATTGCTGTCCACCATCATGTGTA
GGTCCAACAGGCCCAATGGGTCCAAGAGGTAGAACCGGCCCAACAGGTCC
AACGGGTCCAACAGGTCCAGGAGTAGGGGCAACAGGCCCAACAGGACCAA
CCGGTCCGACTGGTCCAACAGGAAATACAGGGAATACAGGAGCAACAGGA
TTAAGAGGTCCAACAGGAGCAACAGGGGCAACAGGCCCAACAGGAGCGAC
AGGAGCTATAGGGTTTGGAGTAACAGGTCCAACAGGCCCAACAGGAGCGA
CAGGAGCAACAGGAGCAGATGGAGTAACAGGTCCAACAGGTCCAACGGGA
GCAACAGGAGCAGATGGAATAACAGGTCCAACAGGAGCAACAGGGGCAAC
AGGATTTGGAGTAACAGGTCCAACAGGCCCAACAGGAGCAACAGGAGTAG
GAGTAACAGGAGCAACAGGATTAATAGGTCCAACAGGAGCGACAGGAACA
CCTGGAGCAACAGGTCCAACAGGGGCAATAGGAGCAACAGGAATAGGAAT
AACAGGTCCAACAGGAGCAACAGGAGCAACAGGGGCAGATGGAGCAACAG
GAGTAACAGGCCCAACAGGCCCAACAGGGGCAACAGGAGCAGATGGAGTA
ACAGGCCCAACAGGAGCAACAGGAGCAACAGGAATAGGAATAACAGGCCC
AACAGGTCCAACAGGAGCAACAGGAATAGGGATAACAGGGGCAACAGGAT
TAATAGGTCCAACAGGGGCAACAGGAACACCTGGAGCAACAGGTCCAACA
GGAGCAACAGGCCCAACAGGAGTAGGAGTAACAGGAGCAACAGGAGCAAC
AGGAGCAACAGGAGCAGACGGAGCAACAGGAGTAACAGGTCCAACAGGAG
CAACAGGGGCAACAGGAGCAAATGGATTAGTAGGCCCAACAGGAGCCACA
GGAGCAGCAGGAACACCTGGAGCAACAGGTCCAACAGGAGCAACAGGCCC
AACAGGAGTAGGAATAACAGGAGCAACAGGGGCAACAGGAGCGACAGGTC
CAACAGGAGCAGATGGAGCAACAGGTCCAACAGGAGCAACAGGAAATACA
GGAGCAGATGGAGTAGCAGGTCCAACAGGAGCAACAGGAAATACAGGAGC
AGATGGAGCAACAGGTCCAACAGGAGCAACAGGGGCAACAGGAGCAGATG
GAGCAACAGGTCCAACAGGAGCAACAGGAGCAACAGGAGTGGCAGGAGCA
ACAGGAGCAACAGGTCCAACAGGAGCAACAGGAGCAGATGGAGCAACAGG
TCCAACAGGAGCAACAGGAGCAACAGGGGCAGATGGAGCAACAGGTCCAA
CAGGAGCAACAGGGGCAACAGGAGTTACAGGAGCAACAGGCCCAACAGGC
CCAACAGGAGCAACAGGAGCAACAGGAGCAACAGGTGCTAGTGCAATAAT
ACCTTTTGCATCAGGTATACCACTATCACTTACAACTATAGCTGGAGGAT
TAGTAGGTACACCTGGATTTGTTGGATTTGGTAGTTCGGCTCCAGGATTA
AGTATAGTTGGTGGAGTAATAGACCTTACAAACGCAGCAGGAACATTGAC
TAACTTTGCATTTTCAATGCCAAGAGATGGAACAATAACATCTATTTCAG
CATACTTCAGTACAACAGCAGCACTTTCACTTGTTGGTTCAACAATTACA
ATTACAGCAACACTTTACCAATCTACTGCACCAAATAACTCATTTACAGC
TGTACCAGGAGCGACAGTTACACTAGCTCCACCACTTACAGGTATATTAT
CAGTTGGTTCAATTTCTAGTGGAATTGTAACAGGATTAAATATAGCAGCA
ACAGCACAAACTCCAGACAGACAGTATGCCATATAA

Furthermore, the polypeptide sequence of C. difficile bclA1 [gi|126697904|ref|YP_{—}001086801.1| exosporium glycoprotein [Clostridium difficile 630]] is provided herein as SEQ ID No:4, as follows:

[SEQ ID No: 4]

MRNIILYLNDDTFISKKYPDKNFSNLDYCLIGSKCSNSFVKEKLITFFKV
RIPDILKDKSILKAELFIHIDSNKNHIFKEKVDIEIKRISEYYNLRTITW
NDRVSMENIRGYLPIGISDTSNYICLNITGTIKAWAMNKYPNYGLALSLN
YPYQILEFTSSRGCNKPYILVTFEDRIIDNCYPKCECPPIRITGPMGPRG
ATGSTGPMGVTGPTGSTGATGSIGPTGPTGNTGATGSIGPTGVTGPTGST
GATGSIGPTGVTGPTGNTGVTGSIGPTGATGPTGNTGVTGSIGPTGVTGP
TGNTGEIGPTGATGPTGVTGSIGPTGATGPTGEIGPTGATGATGSIGPTG
ATGPTGATGVTGEIGPTGEIGPTGATGPTGVTGSIGPTGATGPTGATGEI
GPTGATGPTGVTGSIGPTGATGPTGATGEIGPTGATGPTGVTGEIGPTGA
TGPTGNTGVTGEIGPTGATGPTGNTGVTGEIGPTGATGPTGVTGEIGPTG
NTGATGSIGPTGVTGPTGATGSIGPTGATGATGVTGPTGPTGATGNSSQP
VANFLVNAPSPQTLNNGDAITGWQTIIGNSSSITVDTNGTFTVQENGVYY
ISVSVALQPGSSSINQYSFAILFPILGGKDLAGLTTEPGGGGVLSGYFAG
FLFGGTTFTINNFSSTTVGIRNGQSAGTAATLTIFRIADTVMT

The polypeptide sequence of C. difficile bclA2 [gi|126700850|ref|YP_{—}001089747.1| exosporium glycoprotein [Clostridium difficile 630]] is provided herein as SEQ ID No: 5 as follows:

[SEQ ID No: 5]

MSDISGPSLYQDVGPTGPTGATGPTGPTGPRGATGATGANGITGPTGNTG
ATGANGITGPTGNMGATGPNGTTGSTGPTGNTGATGANGITGPTGNTGAT
GANGITGPTGNKGATGANGITGSTGPTGNTGATGANGITGPTGNTGATGA
TGPTGLTGATGATGANGITGPTGNTGATGANGVTGATGPTGNTGATGPTG
SIGATGATGTTGATGPIGATGATGADGEVGPTGAVGATGPDGLVGPTGPT
GPTGATGANGLVGPTGPTGATGANGLVGPTGATGATGVAGAIGPTGAVGA
TGPTGADGAVGPTGATGATGANGATGPTGAVGATGANGVAGPIGPTGPTG
ENGVAGATGATGATGANGATGPTGAVGATGANGVAGAIGPTGPTGANGAT
GATGATGATGANGATGPTGATGATGVLAANNAQFTVSSSSLVNNTLVTFN
SSFINGTNITFPTSSTINLAVGGIYNVSFGIRATLSLAGFMSITTNFNGV
TQNNFIAKAVNTLTSSDVSVSLSFLVDARAAAVTLSFTFGSGTTGTSAAG
YVSVYRIQ

The polypeptide sequence of C. difficile bclA3 [lcl∥CD3349|bclA3|74729191 putative exosporium glycoprotein] is provided herein as SEQ ID No:6, as follows:

[SEQ ID No: 6]

MLLIMSRNKYFGPFDDNDYNNGYDKYDDCNNGRDDYNSCDCHHCCPPSCV
GPTGPMGPRGRTGPTGPTGPTGPGVGATGPTGPTGPTGPTGNTGNTGATG
LRGPTGATGATGPTGATGAIGFGVTGPTGPTGATGATGADGVTGPTGPTG
ATGADGITGPTGATGATGFGVTGPTGPTGATGVGVTGATGLIGPTGATGT
PGATGPTGAIGATGIGITGPTGATGATGADGATGVTGPTGPTGATGADGV
TGPTGATGATGIGITGPTGPTGATGIGITGATGLIGPTGATGTPGATGPT
GATGPTGVGVTGATGATGATGADGATGVTGPTGATGATGANGLVGPTGAT
GAAGTPGATGPTGATGPTGVGITGATGATGATGPTGADGATGPTGATGNT
GADGVAGPTGATGNTGADGATGPTGATGATGADGATGPTGATGATGVAGA
TGATGPTGATGADGATGPTGATGATGADGATGPTGATGATGVTGATGPTG
PTGATGATGATGASAIIPFASGIPLSLTTIAGGLVGTPGFVGFGSSAPGL
SIVGGVIDLTNAAGTLTNFAFSMPRDGTITSISAYFSTTAALSLVGSTIT
ITATLYQSTAPNNSFTAVPGATVTLAPPLTGILSVGSISSGIVTGLNIAA
TAQTPDRQYAI

Thus, preferably the BclA polypeptide used in the vaccine comprises an amino acid sequence substantially as set out in any one of SEQ ID No:4-6, or a fragment or variant thereof, or is encoded by a nucleic acid sequence substantially as set out in any one of SEQ ID No:1-3, or a fragment or variant thereof.

The inventors have found that C. difficile BclA1 protein produces the optimum results. Accordingly, most preferably the BclA polypeptide used to create the vaccine of the invention comprises C. difficile BclA1. Hence, it is preferred that the BclA polypeptide comprises an amino acid sequence substantially as set out in SEQ ID No:4, or a fragment or variant thereof, or is encoded by a nucleic acid sequence substantially as set out in SEQ ID No:1, or a fragment or variant thereof.

As described in the Examples, the inventors analysed the bclA1 genes in the genome sequences of two ribotype 027 strains, R20291 and CD196, which have a stop codon at position 49, i.e. only the N-terminal 48-50 amino acids of SEQ ID No:1. They have surprisingly shown that the 50% infectious dose (ID₅₀) was higher in mice infected with R20291, a hypervirulent “027” strain. Accordingly, in a preferred embodiment, the vaccine comprises only the N-terminus of the C. difficile BclA polypeptide, which may be represented by SEQ ID No:4-6 or encoded by SEQ ID No:1-3.

It will be appreciated that the full length BclA protein is 693 amino acids in length. The N-terminus, therefore, is described as being amino acids 1-346 of the full length BclA protein. Accordingly, it is preferred that the vaccine comprises only the first 346 amino acids forming the N-terminus of the C. difficile BclA polypeptide. More preferably, the vaccine comprises only the first 300, 200 or 150 amino acids forming the N-terminus of the C. difficile BclA polypeptide. Even more preferably, the vaccine comprises only the first 100 or 50 amino acids forming the N-terminus of the C. difficile BclA polypeptide. As such, the term “fragments” of the BclA polypeptide as used herein can refer to stretches of only the N-terminal amino acids of the protein.

As discussed above, preferably the vaccine comprises only the N-terminus of the C. difficile BclA1 polypeptide, which may be represented by SEQ ID No:4 or is encoded by SEQ ID No:1.

In a preferred embodiment, the BclA1 polypeptide used in the vaccine of the invention comprises amino acid residues 1-48, as set out in SEQ ID No:4.

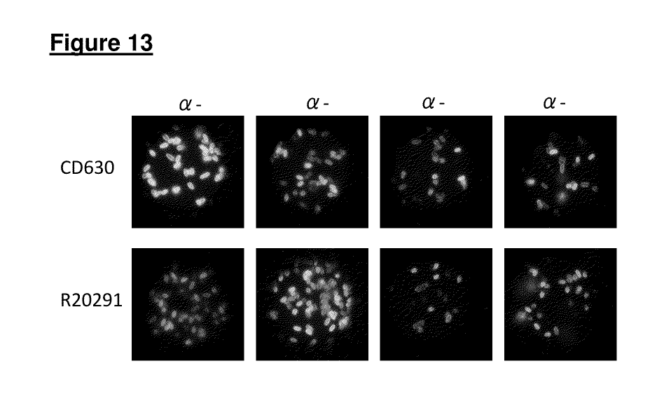

As shown in FIG. 12, the truncated polypeptide sequence of C. difficile bclA1 from the hypervirulent “027” strain, R20291, is provided herein as SEQ ID No:7, as follows:

[SEQ ID No: 7]
MRKIILYLNDDTFISKKYPDKNFSNLDYCLIGSKCSNSFVKEKLITFF

The DNA sequence of this truncated C. difficile bclA1 is provided herein as SEQ ID No:8, as follows:

[SEQ ID No: 8]
ATGAGAAATATTATACTTTATTTAAATGATGATACTTTTATATCTAAAAA
ATATCCAGATAAAAACTTTAGTAATTTAGATTATTGCTTAATAGGAAGTA
AATGTTCAAATAGTTTTGTAAAAGAAAAGTTGATTACTTTTTTTTAA

Hence, in a most preferred embodiment, the BclA polypeptide used in the vaccine of the invention comprises an amino acid sequence substantially as set out in SEQ ID No:7, or is encoded by a nucleic acid sequence substantially as set out in SEQ ID No:8.

Preferably, the vaccine is used to combat various Bacillus spp. infections, including B. anthracis, and B. cereus.

More preferably however, the vaccine is used to combat an infection with Clostridium spp., for example C. difficile, C. perfringens, C. tetani, C. botulinum, C. acetobutylicum, C. cellulolyticum, C. novyi or C. thermocellum. It is most preferred that C. difficile infections are combated, and preferably C. difficile 630.

The vaccine may be prophylactic or therapeutic. Preferably, the vaccine comprises an adjuvant. In the development of a vaccine, it is preferred that the C. difficile BclA1 polypeptide, or a fragment or variant thereof, may be used as an antigen for triggering an immune response in a subject which is to be vaccinated.

Accordingly, in a third aspect, there is provided a C. difficile BclA1 polypeptide, or a fragment or variant thereof, for use in stimulating an immune response in a subject.

In an embodiment, the polypeptide, fragment or variant may be administered directly into a subject to be vaccinated on its own, i.e., just one or more polypeptide comprising an amino acid sequence substantially as set out in any one of SEQ ID No:4-6 or 7, or a fragment or variant thereof. The polypeptide may be administered by injection or mucosally. In another embodiment, the antigen may be delivered to the subject to be vaccinated on a spore. It will be appreciated that administration, into a subject to be vaccinated, of a polypeptide, fragment or variant of the invention (either as a protein or on a spore) will result in the production of corresponding antibodies exhibiting immunospecificity for the polypeptide, fragment or variant, and that these antibodies aid in preventing or combating an infection with Clostridium spp. or Bacillus spp.

The skilled person will appreciate that there are various ways in which a vaccine could be made based on the antigenic fragments represented as any one of SEQ ID No:4-6 or 7, or a fragment or variant thereof. For example, genetically engineered vaccines may be constructed where the heterologous antigen (i.e. the polypeptide, fragment or variant thereof) is fused to a promoter or gene that facilitates expression in a host vector (e.g., a bacterium), or a virus (e.g., Adenovirus). Alternatively, the vaccine may be a DNA molecule based on nucleotide sequences, SEQ ID No's: 1-3 or 8. The vaccine may comprise an excipient, which may act as an adjuvant. Thus, in another embodiment, the antigenic peptide in the vaccine may be combined with a microparticulate adjuvant, for example liposomes, or an immune stimulating complex (ISCOMS). The peptide may be combined with an adjuvant, such as cholera toxin, or a squalene-like molecule.

The examples describe how a suitable vaccine may be prepared. Firstly, C. difficile BclA1 polypeptide, or fragment or variant thereof may be chosen as an antigen against which a subsequently vaccinated subject will produce corresponding antibodies. The DNA sequence of the designated gene encoding the designated protein may then be cloned into a suitable vector to form a genetic construct. Preferably, the C. difficile BclA polypeptide comprises C. difficile BclA1, and most preferably only the N-terminal amino acids thereof, i.e. SEQ ID No: 4 or 7. Preferably, the designated gene is represented by SEQ ID No:1 or 8.

A suitable vector may be pDG364 or pDG1664, which will be known to the skilled person. These vectors enable the ectopic insertion into a suitable host bacterial cell, for example Bacillus subtilis.

The DNA sequence encoding the designated antigen may be inserted into any known target gene from the host bacterial cell (e.g. B. subtilis) that encodes a known protein. The DNA sequence encoding the antigen may be inserted into a multiple cloning site flanked by at least part of an amyE gene, which encodes an alpha amylase. Alternatively, the DNA sequence encoding the antigen may be inserted into a multiple cloning site flanked by at least part of a thrC gene. It will be appreciated that the invention is not limited to insertion at amyE and thrC genes. Insertion into any gene is permissible as long as the growth and sporulation of the host organism is not impaired, i.e. the insertion is functionally redundant.

The thus created genetic construct may be used to transform a vegetative mother cell by double cross-over recombination. Alternatively, the genetic construct may be an integrative vector (e.g. p JH101), which may be used to transform a vegetative mother cell by single cross-over recombination.

The construct may comprise a drug-resistance gene that is selectable in the host cell, for example chloramphenicol resistance. After confirmation of the plasmid clone, the plasmid may then be introduced into a host cell by suitable means. The host may be a B. subtilis cell, which itself produces spores. Transformation may be DNA-mediated transformation or by electroporation. Selection may be achieved by testing for drug resistance carried by the plasmid, and now introduced into the genome.

Expression of the hybrid or chimeric gene may be confirmed using Western blotting and probing of size-fractionated proteins (SDS-PAGE) using antibodies that recognize the introduced antigen (i.e. C. difficile BclA1). If the C. difficile gene fused to the B. subtilis gene is correctly expressed, a new band appears which is recognized only by the antibody, and not normally found in B. subtilis. Other techniques that may be used are immuno-fluorescence microscopy and FACS analysis that can show surface expression of antigens on the host's spore surface.

The resultant spores may be administered to a subject (i.e. vaccination) by an oral, intranasal and/or rectal route. The spores may be administered using one or more of the said oral or intranasal or sub-lingual or rectal routes. Oral administration of spores may be suitably via a tablet a capsule or a liquid suspension or emulsion. Alternatively the spores may be administered in the form of a fine powder or aerosol via a Dischaler® or Turbohaler®. Intranasal administration may suitably be in the form of a fine powder or aerosol nasal spray or modified Dischaler® or Turbohaler®. Sub-lingual administration would be using a fast dissolving film or tablet. Rectal administration may suitably be via a suppository. The spores according to the invention are preferably heat-inactivated prior to administration such that they do not germinate into vegetative cells.

A suitable dosing regime may be used depending on the organism to be vaccinated. For example, for a human subject to be vaccinated, normally three doses (100-500 mg as a tablet or capsule carrying about 2×1010 spores) at 2-week intervals may be used. Blood may be withdrawn for analysis of serum (IgG) responses. Saliva, vaginal fluids or faeces may be taken for analysis of mucosal (secretory IgA) responses. Indirect ELISA may be used to analyse antibody responses in serum and mucosal samples, to gauge the efficacy of the vaccination. The C. difficile BclA1 polypeptide, fragment or variant may be used to treat or prevent relapse/recolonisation of the infection.

In view of the results, the inventors believe that the efficacy of the vaccine of the invention may be further improved by combining toxin A with the C. difficile BclA1 polypeptide, or fragment of variant thereof.

Thus, in one embodiment, the vaccine may further comprise toxin A, or a functional variant or fragment thereof. In another embodiment, the vaccine may further comprise toxin B, or a functional variant or fragment thereof. In yet another embodiment, the vaccine may further comprise toxin A and toxin B, or a functional variant or fragment thereof.

In a fourth aspect, there is provided the vaccine according to the first aspect, for use in treating, ameliorating or preventing an infection with Clostridium spp. or Bacillus spp.

In a fifth aspect, there is provided a method of treating, ameliorating or preventing an infection with Clostridium spp. or Bacillus spp., the method comprising administering, to a subject in need of such treatment, the vaccine according to the first aspect.

The inventors have prepared a series of expression cassettes and vectors for use in the preparation of the vaccine.

Thus, in a sixth aspect, there is provided an isolated genetic construct comprising a nucleotide sequence encoding C. difficile BclA polypeptide, or a fragment or variant thereof.

Preferably, the nucleotide sequence encodes only the N-terminus of the C. difficile BclA1 polypeptide, preferably only the first 300, 200 or 150 amino acids forming the N-terminus of the C. difficile BclA1 polypeptide. Even more preferably, the construct comprises only the first 100, 50 or 48 amino acids forming the N-terminus of the C. difficile BclA1 polypeptide. Hence, the construct may comprise a nucleic acid sequence substantially as set out in any one of SEQ ID No's: 1-3, or 8, or a functional variant or a fragment thereof.

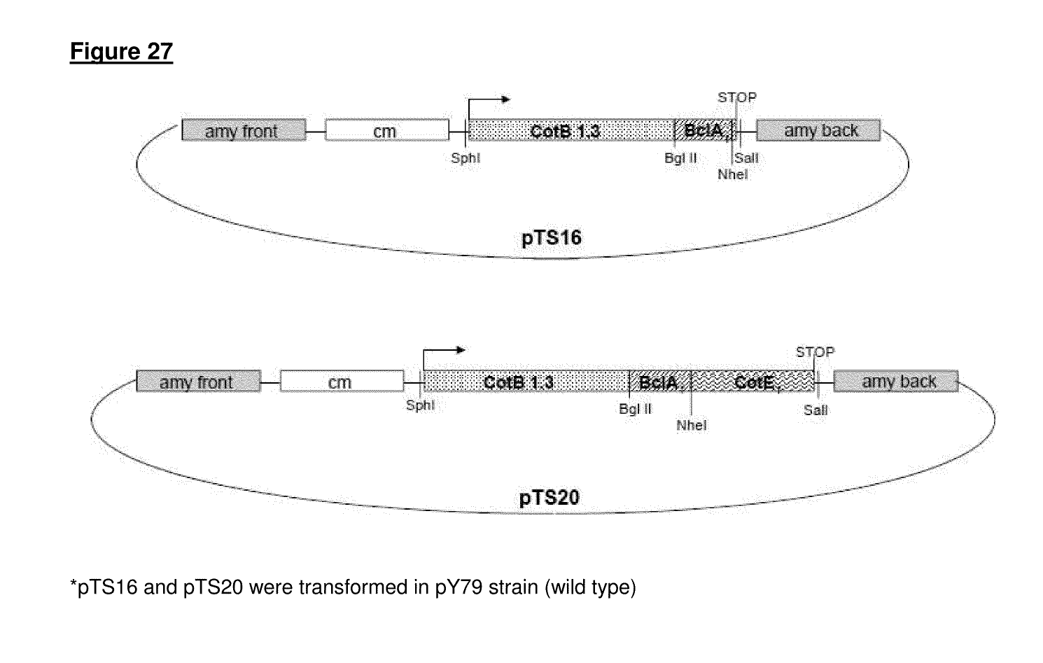

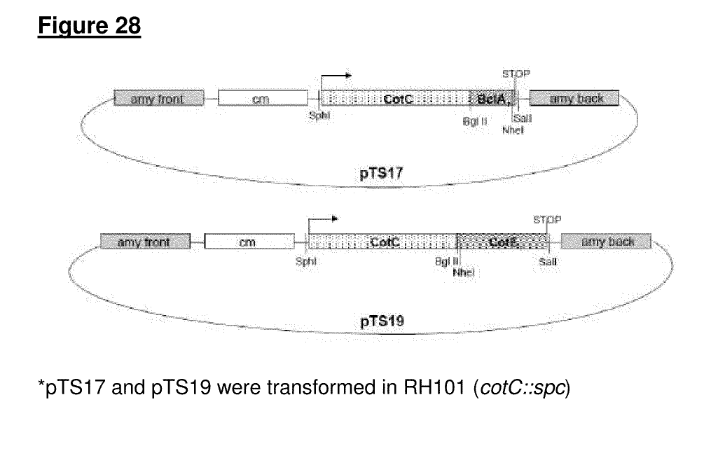

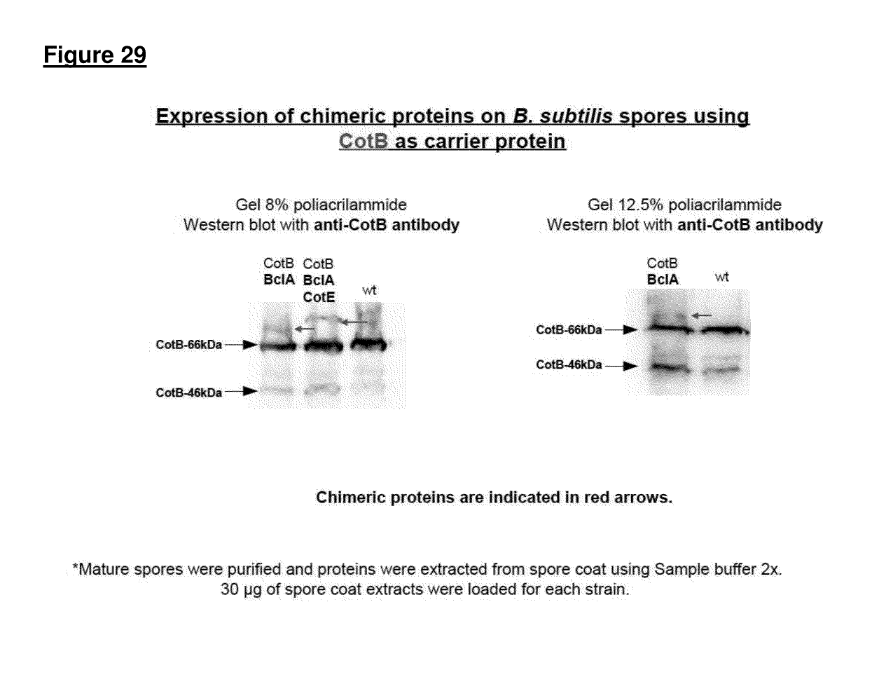

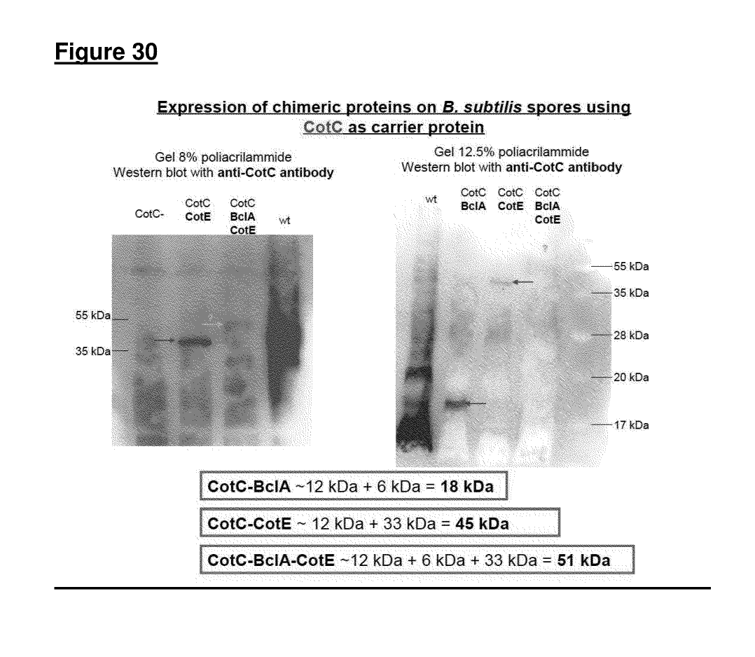

As shown in FIGS. 27 and 28, the inventors have made several constructs (pTS16, pTS20, pTS17 and pTS19) for expressing a range of different fusion proteins comprising the N-terminus of the C. difficile BclA1 polypeptide on the surface of B. subtilis spores (as shown in FIGS. 29 and 30). In one embodiment, the N-terminus of the C. difficile BclA1 polypeptide may be fused to the Bacillus genes CotB and/or CotC, which are known to the skilled person, and are provided to facilitate expression of the encoded fusion protein on the surface of a B. subtilis spore. Hence, preferably the construct further comprises a nucleotide sequence encoding Bacillus subtilis gene CotB and/or CotC or a functional fragment or variant thereof.

The B. subtilis nucleic acid sequence encoding CotB is provided herein as SEQ ID No:9, as follows:

SEQ ID No: 9
ATGAGCAAGAGGAGAATGAAATATCATTCAAATAATGAAATATCGTATTA
TAACTTTTTGCACTCAATGAAAGATAAAATTGTTACTGTATATCGTGGAG
GTCCGGAATCTAAAAAAGGAAAATTAACAGCTGTAAAATCAGATTATATA
GCTTTACAAGCTGAAAAAAAAATAATTTATTATCAGTTGGAGCATGTGAA
AAGTATTACTGAGGATACCAATAATAGCACCACAACAATTGAGACTGAGG
AAATGCTCGATGCTGATGATTTTCATAGCTTAATCGGACATTTAATAAAC
CAATCAGTTCAATTTAACCAAGGGGGTCCGGAATCTAAAAAAGGAAGATT
GGTCTGGCTGGGAGATGATTACGCTGCGTTAAACACAAATGAGGATGGGG
TAGTGTATTTTAATATCCATCACATCAAAAGTATAAGTAAACACGAGCCT
GATTTGAAAATAGAAGAGCAGACGCCAGTTGGAGTTTTGGAAGCTGATGA
TTTAAGCGAGGTTTTTAAGAGTCTGACTCATAAATGGGTTTCAATTAATC
GTGGAGGTCCGGAAGCCATTGAGGGTATCCTTGTAGATAATGCCGACGGC
CATTATACTATAGTGAAAAATCAAGAGGTGCTTCGCATCTATCCTTTTCA
CATAAAAAGCATCAGCTTAGGTCCAAAAGGGTCGTACAAAAAAGAGGATC
AAAAAAATGAACAAAACCAGGAAGACAATAATGATAAGGACAGCAATTCG
TTCATTTCTTCAAAATCATATAGCTCATCAAAATCATCTAAACGATCACT
AAAATCTTCAGATGATCAATCATCCAAATCTGGTCGTTCGTCACGTTCAA
AAAGTTCTTCAAAATCATCTAAACGATCACTAAAATCTTCGGATTATCAA
TCATCCAAATCTGGCCGTTCGTCACGTTCAAAAAGTTCTTCAAAATCATC
TAAACGATCATTAAAATCTTCAGATTATCAATCATCAAAATCATCTAAAC
GATCACCAAGATCTTCAGATTATCAATCATCAAGATCACCAGGCTATTCA
AGTTCAATAAAAAGTTCAGGAAAACAAAAGGAAGATTATAGCTATGAAAC
GATTGTCAGAACGATAGACTATCACTGGAAACGTAAATTT

The B. subtilis nucleic acid sequence encoding CotC is provided herein as SEQ ID No:10, as follows:

SEQ ID No: 10
ATGAAAAATCGGCTCTTTATTTTGATTTGTTTTTGTGTCATCTGTCTTTT
TCTATCATTTGGACAGCCCTTTTTTCCTTCTATGATTTTAACTGTCCAAG
CCGCAAAATCTACTCGCCGTATAATAAAGCGTAGTAAAAATAAAGGAGGA
GTATATAGGGTTATTACAAAAAATACAAAGAAGAGTATTATACGGTCAAA
AAAACGTATTATAAGAAGTATTACGAATATGATAAAAAAGATTATGACTG
TGATTACGACAAAAAATATGATGACTATGATAAAAAATATTATGATCACG
ATAAAAAAGACTATGATTATGTTGTAGAGTATAAAAAGCATAAAAAACAC
TACTAAACGCCATTAACATCTCCTCGTTTTTACTTTCCCCCGGCTATTGC
CGGGTCTTTTTTGTTTGTGCACTATATGTATATTTCTGAAGCTTCCCTTT
CTATGAAAACCTTGGTGACTGAATCTGAAAAAGAATAGTGAATATTTAGT
ACATAGTTTAGACAAA

Preferably, the construct comprises SEQ ID No:9 or 10, or a functional fragment or variant thereof. It will be appreciated that the genetic constructs of the invention are preferably used for expressing chimeras of BClA on the surface of a bacterial spore, preferably B. subtilis.

The inventors have also made a number of genetic constructs based on C. difficile BclA1 and C. difficile CotE using B. subtilis CotB and/or CotC as carrier proteins, and demonstrated that BClA1 (preferably the N-terminus) and CotE (preferably, C-terminus) acts as new antigens that confer some level of protection in animal models.

Hence, in another preferred embodiment, the construct comprises a nucleotide sequence encoding C. difficile gene CotE or a fragment or variant thereof, and most preferably the C-terminus thereof. The nucleic acid sequence encoding C. difficile CotE is provided herein as SEQ ID No:11, as follows:

SEQ ID No: 11
GTGATTTACATGCCAAATTTGCCAAGTTTAGGGTCAAAGGCTCCTGATTT
TAAAGCCAATACAACAAATGGTCCTATTAGACTCTCTGACTATAAGGGTA
ATTGGATTGTTTTATTTTCACATCCTGGTGATTTTACACCAGTTTGTACT
ACAGAATTTTTATGTTTTGCTAAATATTATGACGAATTTAAAAAAAGAAA
TACAGAACTAATTGGTCTAAGTGTTGATAGTAACAGTTCACATTTAGCTT
GGATGTATAATATTTCTTTACTTACAGGTGTAGAAATTCCATTTCCTATT
ATAGAAGATAGAGATATGAGAATTGCCAAGTTATACGGCATGATATCAAA
ACCAATGAGTGATACATCAACTGTTCGCTCTGTATTTATTATAGATAATA
ATCAAATTCTAAGAACGATTCTTTATTATCCACTAACTACAGGAAGAAAT
ATTCCAGAAATACTTAGAATAGTAGATGCACTTCAGACTAGTGATAGAGA
TAATATAGTTACTCCTGCAAACTGGTTTCCTGGAATGCCAGTGATTTTAC
CTTATCCTAAAAACTATAAGGAATTAAAAAATAGAGTTAACAGTTGTAAT
AAGAAATATTCATGTATGGACTGGTACTTATGTTTTGTACCAGATAATTA
TAATGATGAAGAAGTGAGCAAGAAAATTGATAATATTGTAGCTGGAAAAA
AGAACATACTAAAAACATTGAAAATGAATGTAACTGTGAACATGAACATC
ATGACTACCTGAACAAAGCTCTTGATTGTAAACAAGAACACAAGACTGAT
ATTAAAGATGATTGCAATCATGAGAAAAAACATACTAAAAATACTAACAA
AGTTCACAACTCCAAACAAGATAAGTTTAAAGATAAGTCTTGTGATGAAA
TGAATTTTAACTATGACAAAGATGAATCTTGCGACAAAATAAATTCTAGC
TATAACAAAGAAGATAGTAGTTATGAAGATTTCTATAAACATAATTATAA
AAACTACGATTATACTAGCGAAAAAAATACTAAAAAAATAGCTATGAAAA
CTTTAAAAGATTCAAAAAAATTAGTTAGACCACAAATAACAGACCCATAC
AATCCAATAGTTGAAAATGCAAACTGTCCAGATATAAATCCAATTGTAGC
AGAATATGTTCTTGGAAATCCAACTAATGTAGATGCTCAACTATTAGATG
CAGTTATATTTGCTTTTGCTGAGATAGACCAGTCTGGAAATTTGTTTATT
CCTTATCCTAGATTTTTAAACCAATTACTTGCTCTTAAAGGTGAAAAACC
TAGCTTAAAAGTAATTGTAGCTATTGGAGGTTGGGGAGCTGAAGGTTTCT
CTGATGCAGCATTAACACCTACATCTAGATATAATTTTGCAAGACAGGTC
AATCAGATGATAAATGAATATGCTTTAGATGGAATAGATATAGACTGGGA
ATATCCTGGAAGTAGTGCATCTGGAATAACATCAAGACCTCAAGATAGAG
AAAACTTTACACTCTTACTAACTGCCATAAGAGATGTTATAGGGGATGAT
AAATGGCTTAGTGTAGCTGGAACAGGAGATAGAGGATATATAAATTCAAG
TGCTGAAATAGATAAAATAGCTCCTATAATAGATTATTTTAATCTTATGA
GTTATGATTTTACAGCAGGTGAAACAGGCCCAAATGGTAGAAAACATCAA
GCAAATCTTTTTGATTCAGACTTATCTTTGCCAGGATATAGTGTTGATGC
AATGGTGAGAAATCTTGAGAATGCTGGAATGCCTTCTGAAAAAATCCTTC
TCGGTATACCATTTTATGGAAGATTAGGTGCTACTATAACAAGAACTTAT
GATGAGCTTAGAAGGGATTATATAAATAAAAATGGATATGAATATAGATT
TGATAATACTGCTCAAGTTCCGTATTTAGTTAAGGATGGAGATTTTGCAA
TGTCATATGATGATGCTTTATCAATATTCTTAAAAACTCAATATGTTCTT
AGAAATTGTCTAGGTGGTGTATTCTCATGGACATCAACTTATGACCAAGC
AAATATACTGGCTAGAACCATGTCTATTGGTATAAATGACCCTGAAGTAT
TAAAAGAAGAACTTGAAGGTATTTATGGGCAATTCTAA

The amino acid sequence of C. difficile CotE is provided herein as SEQ ID No:12, as follows:

SEQ ID No. 12
MIYMPNLPSLGSKAPDFKANTTNGPIRLSDYKGNWIVLFSHPGDFTPVCT
TEFLCFAKYYDEFKKRNTELIGLSVDSNSSHLAWMYNISLLTGVEIPFPI
IEDRDMRIAKLYGMISKPMSDTSTVRSVFIIDNNQILRTILYYPLTTGRN
IPEILRIVDALQTSDRDNIVTPANWFPGMPVILPYPKNYKELKNRVNSCN
KKYSCMDWYLCFVPDNYNDEEVSKKIDNTCSWKKEHTKNIENECNCEHEH
HDYLNKALDCKQEHKTDIKDDCNHEKKHTKNTNKVHNSKQDKFKDKSCDE
MNFNYDKDESCDKINSSYNKEDSSYEDFYKHNYKNYDYTSEKNTKKIAMK
TLKDSKKLVRPQITDPYNPIVENANCPDINPIVAEYVLGNPTNVDAQLLD
AVIFAFAEIDQSGNLFIPYPRFLNQLLALKGEKPSLKVIVAIGGWGAEGF
SDAALTPTSRYNFARQVNQMINEYALDGIDIDWEYPGSSASGITSRPQDR
ENFTLLLTAIRDVIGDDKWLSVAGTGDRGYINSSAEIDKIAPIIDYFNLM
SYDFTAGETGPNGRKHQANLFDSDLSLPGYSVDAMVRNLENAGMPSEKIL
LGIPFYGRLGATITRTYDELRRDYINKNGYEYRFDNTAQVPYLVKDGDFA
MSYDDALSIFLKTQYVLRNCLGGVFSWTSTYDQANILARTMSIGINDPEV
LKEELEGIYGQF

Thus, preferably the construct comprises SEQ ID No: n or encodes SEQ ID No:12, or a functional fragment or variant thereof. Preferably, the C-terminus of CotE is formed by the last 150, 100 or 50 amino acids.

In one embodiment, the construct of the sixth aspect may comprise a nucleotide sequence encoding C. difficile gene BclA and B. subtilis CotB or a fragment or variant thereof. The nucleic acid sequence (harboured in a vector called pTS16) is provided herein as SEQ ID No:13, as follows:

SEQ ID No. 13
ATGAGCAAGAGGAGAATGAAATATCATTCAAATAATGAAATATCGTATTA
TAACTTTTTGCACTCAATGAAAGATAAAATTGTTACTGTATATCGTGGAG
GTCCGGAATCTAAAAAAGGAAAATTAACAGCTGTAAAATCAGATTATATA
GCTTTACAAGCTGAAAAAAAAATAATTTATTATCAGTTGGAGCATGTGAA
AAGTATTACTGAGGATACCAATAATAGCACCACAACAATTGAGACTGAGG
AAATGCTCGATGCTGATGATTTTCATAGCTTAATCGGACATTTAATAAAC
CAATCAGTTCAATTTAACCAAGGGGGTCCGGAATCTAAAAAAGGAAGATT
GGTCTGGCTGGGAGATGATTACGCTGCGTTAAACACAAATGAGGATGGGG
TAGTGTATTTTAATATCCATCACATCAAAAGTATAAGTAAACACGAGCCT
GATTTGAAAATAGAAGAGCAGACGCCAGTTGGAGTTTTGGAAGCTGATGA
TTTAAGCGAGGTTTTTAAGAGTCTGACTCATAAATGGGTTTCAATTAATC
GTGGAGGTCCGGAAGCCATTGAGGGTATCCTTGTAGATAATGCCGACGGC
CATTATACTATAGTGAAAAATCAAGAGGTGCTTCGCATCTATCCTTTTCA
CATAAAAAGCATCAGCTTAGGTCCAAAAGGGTCGTACAAAAAAGAGGATC
AAAAAAATGAACAAAACCAGAAGACAATAATGATAAGGACAGCAATTCGT
TCATTTCTTCAAAATCATATAGCTCATCAAAATCATCTAAACGATCACTA
AAATCTTCAGATGATCAATCATCCAAATCTGGTCGTTCGTCACGTTCAAA
AAGTTCTTCAAAATCATCTAAACGATCACTAAAATCTTCGGATTATCAAT
CATCCAAATCTGGCCGTTCGTCACGTTCAAAAAGTTCTTCAAAATCATCT
AAACGATCATTAAAATCTTCAGATTATCAATCATCAAAATCATCTAAACG
ATCACCAAGATCTATGAGAAATATTATACTTTATTTAAATGATGATACTT
TTATATCTAAAAAATATCCAGATAAAAACTTTAGTAATTTAGATTATTGC
TTAATAGGAAGTAAATGTTCAAATAGTTTTGTAAAAGAAAAGTTGATTAC
TTTTTTTGCTAGCTAATAA

The construct may comprise an amino acid sequence which is provided herein as SEQ ID No:14, as follows:

SEQ ID No. 14
MSKRRMKYHSNNEISYYNFLHSMKDKIVTVYRGGPESKKGKLTAVKSDYI
ALQAEKKIIYYQLEHVKSITEDTNNSTTTIETEEMLDADDFHSLIGHLIN
QSVQFNQGGPESKKGRLVWLGDDYAALNTNEDGVVYFNIHHIKSISKHEP
DLKIEEQTPVGVLEADDLSEVFKSLTHKWVSINRGGPEAIEGILVDNADG
HYTIVKNQEVLRIYPFHIKSISLGPKGSYKKEDQKNEQNQEDNNDKDSNS
FISSKSYSSSKSSKRSLKSSDDQSSKSGRSSRSKSSSKSSKRSLKSSDYQ
SSKSGRSSRSKSSSKSSKRSLKSSDYQSSKSSKRSPRSMRNIILYLNDDT
FISKKYPDKNFSNLDYCLIGSKCSNSFVKEKLITFFAS

In another embodiment, the construct of the sixth aspect may comprise a nucleotide sequence encoding C. difficile gene BclA, B. subtilis CotB and C. difficile CotE, or a fragment or variant thereof. The nucleic acid sequence (harboured in a vector called pTS20) is provided herein as SEQ ID No:15, as follows:

SEQ ID No. 15
ATGAGCAAGAGGAGAATGAAATATCATTCAAATAATGAAATATCGTATTA
TAACTTTTTGCACTCAATGAAAGATAAAATTGTTACTGTATATCGTGGAG
GTCCGGAATCTAAAAAAGGAAAATTAACAGCTGTAAAATCAGATTATATA
GCTTTACAAGCTGAAAAAAAAATAATTTATTATCAGTTGGAGCATGTGAA
AAGTATTACTGAGGATACCAATAATAGCACCACAACAATTGAGACTGAGG
AAATGCTCGATGCTGATGATTTTCATAGCTTAATCGGACATTTAATAAAC
CAATCAGTTCAATTTAACCAAGGGGGTCCGGAATCTAAAAAAGGAAGATT
GGTCTGGCTGGGAGATGATTACGCTGCGTTAAACACAAATGAGGATGGGG
TAGTGTATTTTAATATCCATCACATCAAAAGTATAAGTAAACACGAGCCT
GATTTGAAAATAGAAGAGCAGACGCCAGTTGGAGTTTTGGAAGCTGATGA
TTTAAGCGAGGTTTTTAAGAGTCTGACTCATAAATGGGTTTCAATTAATC
GTGGAGGTCCGGAAGCCATTGAGGGTATCCTTGTAGATAATGCCGACGGC
CATTATACTATAGTGAAAAATCAAGAGGTGCTTCGCATCTATCCTTTTCA
CATAAAAAGCATCAGCTTAGGTCCAAAAGGGTCGTACAAAAAAGAGGATC
AAAAAAATGAACAAAACCAGGAAGACAATAATGATAAGGACAGCAATTCG
TTCATTTCTTCAAAATCATATAGCTCATCAAAATCATCTAAACGATCACT
AAAATCTTCAGATGATCAATCATCCAAATCTGGTCGTTCGTCACGTTCAA
AAAGTTCTTCAAAATCATCTAAACGATCACTAAAATCTTCGGATTATCAA
TCATCCAAATCTGGCCGTTCGTCACGTTCAAAAAGTTCTTCAAAATCATC
TAAACGATCATTAAAATCTTCAGATTATCAATCATCAAAATCATCTAAAC
GATCACCAAGATCTATGAGAAATATTATACTTTATTTAAATGATGATACT
TTTATATCTAAAAAATATCCAGATAAAAACTTTAGTAATTTAGATTATTG
CTTAATAGGAAGTAAATGTTCAAATAGTTTTGTAAAAGAAAAGTTGATTA
CTTTTTTTGCTAGCCCAACTAATGTAGATGCTCAACTATTAGATGCAGTT
ATATTTGCTTTTGCTGAGATAGACCAGTCTGGAAATTTGTTTATTCCTTA
TCCTAGATTTTTAAACCAATTACTTGCTCTTAAAGGTGAAAAACCTAGCT
TAAAAGTAATTGTAGCTATTGGAGGTTGGGGAGCTGAAGGTTTCTCTGAT
GCAGCATTAACACCTACATCTAGATATAATTTTGCAAGACAGGTCAATCA
GATGATAAATGAATATGCTTTAGATGGAATAGATATAGACTGGGAATATC
CTGGAAGTAGTGCATCTGGAATAACATCAAGACCTCAAGATAGAGAAAAC
TTTACACTCTTACTAACTGCCATAAGAGATGTTATAGGGGATGATAAATG
GCTTAGTGTAGCTGGAACAGGAGATAGAGGATATATAAATTCAAGTGCTG
AAATAGATAAAATAGCTCCTATAATAGATTATTTTAATCTTATGAGTTAT
GATTTTACAGCAGGTGAAACAGGCCCAAATGGTAGAAAACATCAAGCAAA
TCTTTTTGATTCAGACTTATCTTTGCCAGGATATAGTGTTGATGCAATGG
TGAGAAATCTTGAGAATGCTGGAATGCCTTCTGAAAAAATCCTTCTCGGT
ATACCATTTTATGGAAGATTAGGTGCTACTATAACAAGAACTTATGATGA
GCTTAGAAGGGATTATATAAATAAAAATGGATATGAATATAGATTTGATA
ATACTGCTCAAGTTCCGTATTTAGTTAAGGATGGAGATTTTGCAATGTCA
TATGATGATGCTTTATCAATATTCTTAAAAACTCAATATGTTCTTAGAAA
TTGTCTAGGTGGTGTATTCTCATGGACATCAACTTATGACCAATAA

The construct may comprise an amino acid sequence which is provided herein as SEQ ID No: 16, as follows:

SEQ ID No. 16
MSKRRMKYHSNNEISYYNFLHSMKDKIVTVYRGGPESKKGKLTAVKSDYI
ALQAEKKIIYYQLEHVKSITEDTNNSTTTIETEEMLDADDFHSLIGHLIN
QSVQFNQGGPESKKGRLVWLGDDYAALNTNEDGVVYFNIHHIKSISKHEP
DLKIEEQTPVGVLEADDLSEVFKSLTHKWVSINRGGPEAIEGILVDNADG
HYTIVKNQEVLRIYPFHIKSISLGPKGSYKKEDQKNEQNQEDNNDKDSNS
FISSKSYSSSKSSKRSLKSSDDQSSKSGRSSRSKSSSKSSKRSLKSSDYQ
SSKSGRSSRSKSSSKSSKRSLKSSDYQSSKSSKRSPRSMRNIILYLNDDT
FISKKYPDKNFSNLDYCLIGSKCSNSFVKEKLITFFASPTNVDAQLLDAV
IFAFAEIDQSGNLFIPYPRFLNQLLALKGEKPSLKVIVAIGGWGAEGFSD
AALTPTSRYNFARQVNQMINEYALDGIDIDWEYPGSSASGITSRPQDREN
FTLLLTAIRDVIGDDKWLSVAGTGDRGYINSSAEIDKIAPIIDYFNLMSY
DFTAGETGPNGRKHQANLFDSDLSLPGYSVDAMVRNLENAGMPSEKILLG
IPFYGRLGATITRTYDELRRDYINKNGYEYRFDNTAQVPYLVKDGDFAMS
YDDALSIFLKTQYVLRNCLGGVFSWTSTYDQ

It will be appreciated that SEQ ID No's 13-16 involve the use of B. subtilis CotB as a carrier. However, as mentioned above, B. subtilis CotC may also be used as a carrier.

Thus, in one embodiment, the construct of the sixth aspect may comprise a nucleotide sequence encoding C. difficile gene BclA and B. subtilis CotC or a fragment or variant thereof. The nucleic acid sequence (harboured in a vector called pTS17) is provided herein as SEQ ID No:17, as follows:

SEQ ID No. 17
ATGAAAAATCGGCTCTTTATTTTGATTTGTTTTTGTGTCATCTGTCTTTT
TCTATCATTTGGACAGCCCTTTTTTCCTTCTATGATTTTAACTGTCCAAG
CCGCAAAATCTACTCGCCGTATAATAAAGCGTAGTAAAAATAAAGGAGGA
GTATATATGGGTTATTACAAAAAATACAAAGAAGAGTATTATACGGTCAA
AAAAACGTATTATAAGAAGTATTACGAATATGATAAAAAAGATTATGACT
GTGATTACGACAAAAAATATGATGACTATGATAAAAAATATTATGATCAC
GATAAAAAAGACTATGATTATGTTGTAGAGTATAAAAAGCATAAAAAACA
CTACAGATCTATGAGAAATATTATACTTTATTTAAATGATGATACTTTTA
TATCTAAAAAATATCCAGATAAAAACTTTAGTAATTTAGATTATTGCTTA
ATAGGAAGTAAATGTTCAAATAGTTTTGTAAAAGAAAAGTTGATTACTTT
TTTTGCTAGCTAATAA

The construct may comprise an amino acid sequence which is provided herein as SEQ ID No: 18, as follows:

SEQ ID No. 18
MKNRLFILICFCVICLFLSFGQPFFPSMILTVQAAKSTRRIIKRSKNKGG
VYMGYYKKYKEEYYTVKKTYYKKYYEYDKKDYDCDYDKKYDDYDKKYYDH
DKKDYDYVVEYKKHKKHYRSMRNIILYLNDDTFISKKYPDKNFSNLDYCL
IGSKCSNSFVKEKLITFFAS

It will be appreciated that CotE (preferably the C-terminus) and BClA1 (preferably the N-terminus) of C. difficile may be delivered mucosally (e.g. by oral dosing) using heat stable bacterial spores and provide decolonisation of C. difficile. They would achieve this by inducing mucosal (secretory IgA) responses that prevent spores of C. difficile from colonising the gut epithelium. Antibodies to BclA1 and CotE are surprisingly protective. The inventors have shown that the use of spores displaying BclA1, CotE or BclA1-CotE confers greater levels of protection (using toxin production and colonisation as indicative markers of C. difficile infection) when administered in combination with B. subtilis spores expressing a C-terminal fragment of toxin A (TcdA26-39) that use of spores expressing TcdA26=39 alone.

The vaccine may therefore comprise spores expressing one or more of: toxin A, BclA1, CotE, BClA1-CotE fusion, or a functional fragments or variants thereof. Therefore, most preferably the vaccine of the first aspect comprises a combination of spores expressing TcdA26-39 and spores expressing BclA1 (preferably N-terminus), CotE (preferably C-terminus) or BclA1-CotE (fusion).

In the case of BclA1, the inventors have identified the utility of the N-terminal domain as being important for protection. They have also shown that the BclA1 and CotE domains may be combined as chimeras and be expressed as fusions to two protein components of the Bacillus subtilis spore coat, CotB and CotC. This teaches us that the N-terminal domain of BclA1 and the C-terminal domain of CotE can be stably expressed on the spore surface (fused to spore coat proteins) and be expressed together as a BclA1-CotE chimeras. Since the N-terminal domain of BclA1 is conserved among all C. difficile strains this region is important in a vaccine formulation.

Genetic constructs of the invention may be in the form of an expression cassette, which may be suitable for expression of the encoded polypeptide in a host cell. The genetic construct may be introduced in to a host cell without it being incorporated in a vector. For instance, the genetic construct, which may be a nucleic acid molecule, may be incorporated within a liposome or a virus particle. Alternatively, a purified nucleic acid molecule (e.g. histone-free DNA, or naked DNA) may be inserted directly into a host cell by suitable means, e.g. direct endocytotic uptake. The genetic construct may be introduced directly in to cells of a host subject (e.g. a bacterial cell, such as Bacillus) by transfection, infection, electroporation, microinjection, cell fusion, protoplast fusion or ballistic bombardment. Alternatively, genetic constructs of the invention may be introduced directly into a host cell using a particle gun. Alternatively, the genetic construct may be harboured within a recombinant vector, for expression in a suitable host cell.

Therefore, in a seventh aspect, there is provided a recombinant vector comprising the genetic construct according to the sixth aspect.

The recombinant vector may be a plasmid, cosmid or phage. Such recombinant vectors are useful for transforming host cells with the genetic construct of the sixth aspect, and for replicating the expression cassette therein. The skilled technician will appreciate that genetic constructs of the invention may be combined with many types of backbone vector for expression purposes. Examples of suitable backbone vectors include pDG364 (see FIGS. 27 and 28). Recombinant vectors may include a variety of other functional elements including a suitable promoter to initiate gene expression. For instance, the recombinant vector may be designed such that it autonomously replicates in the cytosol of the host cell. In this case, elements which induce or regulate DNA replication may be required in the recombinant vector. Alternatively, the recombinant vector may be designed such that it integrates into the genome of a host cell, for example when the backbone vector is pJH101. In this case, DNA sequences which favour targeted integration (e.g. by homologous recombination) are envisaged.

The recombinant vector may also comprise DNA coding for a gene that may be used as a selectable marker in the cloning process, i.e. to enable selection of cells that have been transfected or transformed, and to enable the selection of cells harbouring vectors incorporating heterologous DNA. For example, chloramphenicol (cm) resistance is envisaged. Alternatively, the selectable marker gene may be in a different vector to be used simultaneously with vector containing the gene of interest. The vector may also comprise DNA involved with regulating expression of the coding sequence, or for targeting the expressed polypeptide to a certain part of the host cell.

Preferred vectors of the invention are shown in FIGS. 28 and 29.

In an eighth aspect, there is provided a host cell comprising the genetic construct according to the sixth aspect, or the recombinant vector according to the seventh aspect.

The host cell may preferably be a bacterial cell, for example Bacillus subtilis. Alternatively, the host cell may be an animal cell, for example a mouse or rat cell. It is most preferred that the host cell is not a human cell. The host cell may be transformed with genetic constructs or vectors according to the invention, using known techniques. Suitable means for introducing the genetic construct into the host cell will depend on the type of cell.

In a ninth aspect, there is provided a transgenic host organism comprising at least one host cell according to the eighth aspect.

The genome of the host cell or the transgenic host organism of the invention may comprise a nucleic acid sequence encoding a C. difficile BclA polypeptide, variant or fragment according to the invention, preferably the N-terminus of BClA1. The host organism may be a multicellular organism, which is preferably non-human. For example, the host organism may be a mouse or rat. The host may be a bacterium, preferably Bacillus, most preferably B. subtilis.

It will be appreciated that vaccines and medicaments according to the invention may be used in a monotherapy, for treating, ameliorating or preventing an infection with Clostridium spp. or Bacillus spp. Alternatively, vaccines and medicaments according to the invention may be used as an adjunct to, or in combination with, known therapies for treating, ameliorating, or preventing infections with Clostridium spp. or Bacillus spp. For example, the vaccine may be used in combination with known agents for treating Clostridium spp. or Bacillus spp. infections. Antibiotics used for C. difficile include clindamycin, vancomycin, and metrodinazole. Probiotics used for C. difficile include Lactobacilli and Bifidobacteria.

The vaccines according to the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given, and preferably enables delivery of the agents across the blood-brain barrier.

Medicaments comprising vaccines of the invention may be used in a number of ways. For instance, oral administration may be required, in which case the polypeptides may be contained within a composition that may, for example, be ingested orally in the form of a tablet, capsule or liquid. Compositions comprising vaccines of the invention may be administered by inhalation (e.g. intranasally). Compositions may also be formulated for topical use. For instance, creams or ointments may be applied to the skin.

Vaccines according to the invention may also be incorporated within a slow- or delayed-release device. Such devices may, for example, be inserted on or under the skin, and the medicament may be released over weeks or even months. The device may be located at least adjacent the treatment site. Such devices may be particularly advantageous when long-term treatment with vaccines used according to the invention is required and which would normally require frequent administration (e.g. at least daily injection).

In a preferred embodiment, vaccines and medicaments according to the invention may be administered to a subject by injection into the blood stream or directly into a site requiring treatment. Injections may be intravenous (bolus or infusion) or subcutaneous (bolus or infusion), or intradermal (bolus or infusion).

It will be appreciated that the amount of the vaccine that is required is determined by its biological activity and bioavailability, which in turn depends on the mode of administration, the physiochemical properties of the polypeptides, vaccine and medicament, and whether it is being used as a monotherapy or in a combined therapy. The frequency of administration will also be influenced by the half-life of the vaccine within the subject being treated. Optimal dosages to be administered may be determined by those skilled in the art, and will vary with the particular agent in use, the strength of the pharmaceutical composition, the mode of administration, and the advancement of the bacterial infection. Additional factors depending on the particular subject being treated will result in a need to adjust dosages, including subject age, weight, gender, diet, and time of administration.

Generally, a daily dose of between 0.001 μg/kg of body weight and 10 mg/kg of body weight of vaccine according to the invention may be used for treating, ameliorating, or preventing bacterial infection, depending upon which vaccine is used. More preferably, the daily dose is between 0.01 μg/kg of body weight and 1 mg/kg of body weight, more preferably between 0.1 μg/kg and 100 μg/kg body weight, and most preferably between approximately 0.1 μg/kg and 10 μg/kg body weight.

The vaccine may be administered before, during or after onset of the bacterial infection. Daily doses may be given as a single administration (e.g. a single daily injection). Alternatively, the vaccine may require administration twice or more times during a day. As an example, vaccines may be administered as two (or more depending upon the severity of the bacterial infection being treated) daily doses of between 0.07 μg and 700 mg (i.e. assuming a body weight of 70 kg). A patient receiving treatment may take a first dose upon waking and then a second dose in the evening (if on a two dose regime) or at 3- or 4-hourly intervals thereafter. Alternatively, a slow release device may be used to provide optimal doses of vaccines according to the invention to a patient without the need to administer repeated doses. Known procedures, such as those conventionally employed by the pharmaceutical industry (e.g. in vivo experimentation, clinical trials, etc.), may be used to form specific formulations of the vaccines according to the invention and precise therapeutic regimes (such as daily doses of the polypeptides and the frequency of administration).

A “subject” may be a vertebrate, mammal, or domestic animal. Hence, vaccines according to the invention may be used to treat any mammal, for example livestock (e.g. a horse), pets, or may be used in other veterinary applications. Most preferably, the subject is a human being.

As well as being useful for making a vaccine, the inventors have also demonstrated that C. difficile BclA (and preferably the N-terminus of C. difficile BclA1) can be used as an effective target for detecting the presence of C. difficile in an unknown sample, and therefore diagnosing infections with this bacterium. Furthermore, the inventors have found that there are a number of orthologues of the C. difficile BclA in other spore forming bacterial species. Therefore, the inventors believe that, in addition to C. difficile, BclA may also be used as a target for detecting the presence of Clostridium spp. or Bacillus spp. spores present in a sample, and diagnosing infections with these bacteria.

Therefore, according to a tenth aspect, there is provided use of a C. difficile BclA polypeptide, or a fragment or variant thereof, in the detection of Clostridium spp. or Bacillus spp. in a sample.

In an eleventh aspect, there is provided a Clostridium spp. or Bacillus spp. detection kit, the kit comprising detection means arranged, in use, to detect, in a sample, the presence of a C. difficile BclA polypeptide, or a fragment or variant thereof, wherein detection of the polypeptide, fragment or variant thereof signifies the presence of Clostridium spp. or Bacillus spp.

In a twelfth aspect, there is provided a method of detecting Clostridium spp. or Bacillus spp., the method comprising the steps of detecting, in a sample, for the presence of a C. difficile BclA polypeptide, or a fragment or variant thereof, wherein detection of the polypeptide, fragment or variant thereof signifies the presence of Clostridium spp. or Bacillus spp.

Preferably, the C. difficile BclA polypeptide is as defined in accordance with the previous aspects. Preferably, only the N-terminus of the C. difficile BclA polypeptide is used, more preferably only the first 300, 200, 150, 100 or 50 amino acids forming the N-terminus of the C. difficile BclA polypeptide, preferably BclA1 (i.e. SEQ ID No. 1 and 4).

The use, kit and/or method may each be used to detect for the presence of a spore of Clostridium spp. or Bacillus spp. in the sample.

The use, kit and/or method may each be used to detect a wide range of Clostridium spp. in the sample, for example C. difficile, C. perfringens, C. tetani, C. botulinum, C. acetobutylicum, C. cellulolyticum, C. novyi or C. thermocellum. It is preferred that C. difficile may be detected, and preferably C. difficile 630.

The use, kit and/or method may each be used to detect a wide range of Bacillus spp. in the sample, for example B. anthracis or B. cereus. The use, kit and/or method may be used to detect B. anthracis, which has an exosporium, and proteins exhibiting homology with C. difficile proteins.

The sample may be obtained from a subject suspected of being infected with Clostridium spp. or Bacillus spp., for example a patient in a hospital. The sample may be a sample of a bodily fluid into which a Clostridium spp. or Bacillus spp. infection could result. For example, the sample may comprise blood, urine, saliva or vaginal fluid. C. difficile is normally diagnosed from faeces, and so the sample may be a faecal sample. A suitable method for sample preparation may be used prior to carrying out the detection method thereon.

The detection means is preferably arranged to bind to a C. difficile BclA polypeptide, or a fragment or variant thereof, and thereby form a complex, which complex can be detected, thereby signifying the presence of Clostridium spp. or Bacillus spp. For example, the detection means may comprise a polyclonal or monoclonal antibody, which may be prepared using techniques known to the skilled person. Polyclonal antisera/antibodies and/or monoclonal antisera/antibodies may first be made against the BclA polypeptide of the invention acting as an antigen, i.e. the C. difficile or Bacillus spp. spore coat protein.

The test sample, potentially containing Clostridium spp. (preferably C. difficile) or Bacillus spp., may then be contacted with the detection means in order to allow a complex to form, and this complex may then be subsequently evaluated using an appropriate method to diagnose the presence or absence of the antigen (i.e. any of SEQ ID No.4-7). A positive detection of Clostridium spp. or Bacillus spp. spores in the sample will occur if they display and carry the relevant antigens that react with BclA (exhibiting immunospecificity with BclA).

The method or kit of the invention may comprise a positive control and/or a negative control. Thus, the test sample may be compared to the positive and/or negative control, in order to determine whether or not the sample is infected with Clostridium spp. or Bacillus spp. The positive control may comprise any of SEQ ID No.4-7, or a fragment or variant thereof.

Several embodiments of the kit have been developed. In one embodiment, the kit may comprise latex agglutination. An antibody may be contacted with a test sample, and a positive reaction may be seen by agglutination of a complex comprising BclA antibody and the BclA antigen. The antibody may be first bound to a support structure, for example a latex bead. In the presence of antigen, the support structures will form clumps or coagulate.

In a second embodiment, the kit may comprise lateral flow. The antibodies may be applied as a thin strip to a suitable membrane. The strip may be pre-soaked with a reagent that, in the presence of the antigen-antibody complex, should one form, produced a detectable result, for example a colour change or reaction that is visible to the naked eye. The sample (containing Clostridium spp. or Bacillus spp. antigen) may be applied as a drop to one end of the strip. As the aqueous sample diffuses through the membrane, it passes through a band of membrane carrying the reagent. As it moves further, it reaches the band carrying the antibody where it will complex with the antibody and form a defined strip which, in the presence of the reagent (e.g. a colour compound), will be visible to the naked eye as a thin line.

In a third embodiment, the kit may comprise a “dipstick”. Antibody may first be applied to one end of a support surface or “dipstick”. When the pre-coated support is then spotted onto a test sample, potentially containing Clostridium spp. or Bacillus spp., the antigen-antibody complex will be visualized using a secondary substrate.

Other techniques can be used to detect BclA protein described herein, all of which rely on the detection of antibody-antigen complexes, for example surface plasmon resonance (SPR), optical methods, fluorescence-based methods or magnetic particles. Another technique which may be used includes ELISA. In this embodiment, the sample may be first diluted, and ELISA may then be used to detect antigen-antibody binding between the BclA antibodies and BclA proteins on the spore coat of any Clostridium spp. or Bacillus spp. infecting the sample. By dilution of the sample, a good indication of the quantity of antigen on the infecting bacteria in the test sample can be determined.

It will be appreciated that the invention extends to any nucleic acid or peptide or variant, derivative or analogue thereof, which comprises substantially the amino acid or nucleic acid sequences of any of the sequences referred to herein, including functional variants or functional fragments thereof. The terms “substantially the amino acid/nucleotide/peptide sequence”, “functional variant” and “functional fragment”, can be a sequence that has at least 40% sequence identity with the amino acid/nucleotide/peptide sequences of any one of the sequences referred to herein, for example 40% identity with the sequence identified as SEQ ID No: 4-7 (i.e. BClA proteins or truncation thereof) or the nucleotide identified as SEQ ID No: 1-3, 8 (i.e. BClA genes), or 40% identity with the polypeptide identified as SEQ ID No: 4-7 (i.e. BClA protein) or the nucleotide identified as SEQ ID No: 1-3, 8 (i.e. BClA gene), and so on.

Amino acid/polynucleotide/polypeptide sequences with a sequence identity which is greater than 50%, more preferably greater than 65%, 70%, 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90%, 92%, 95%, 97%, 98%, and most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (iv) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps but excluding overhangs. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence which hybridizes to the sequences shown in SEQ ID No's: 1-3, 8 or their complements under stringent conditions. By stringent conditions, we mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the sequences shown in SEQ ID No: 4-7.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying diagrammatic drawings, in which:—

FIG. 1 is a genome map showing the location of the three C. difficile bclA genes, bclA/, bclA2 and bclA3;

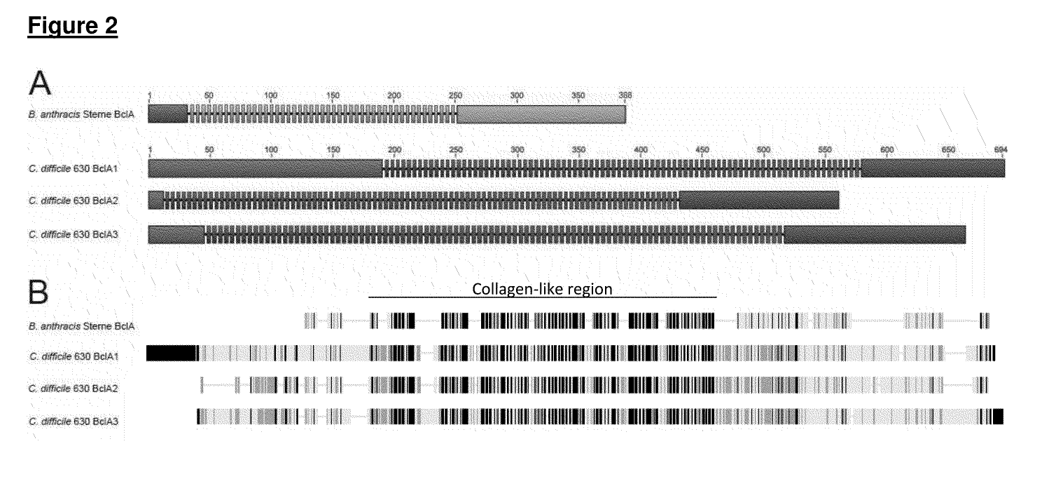

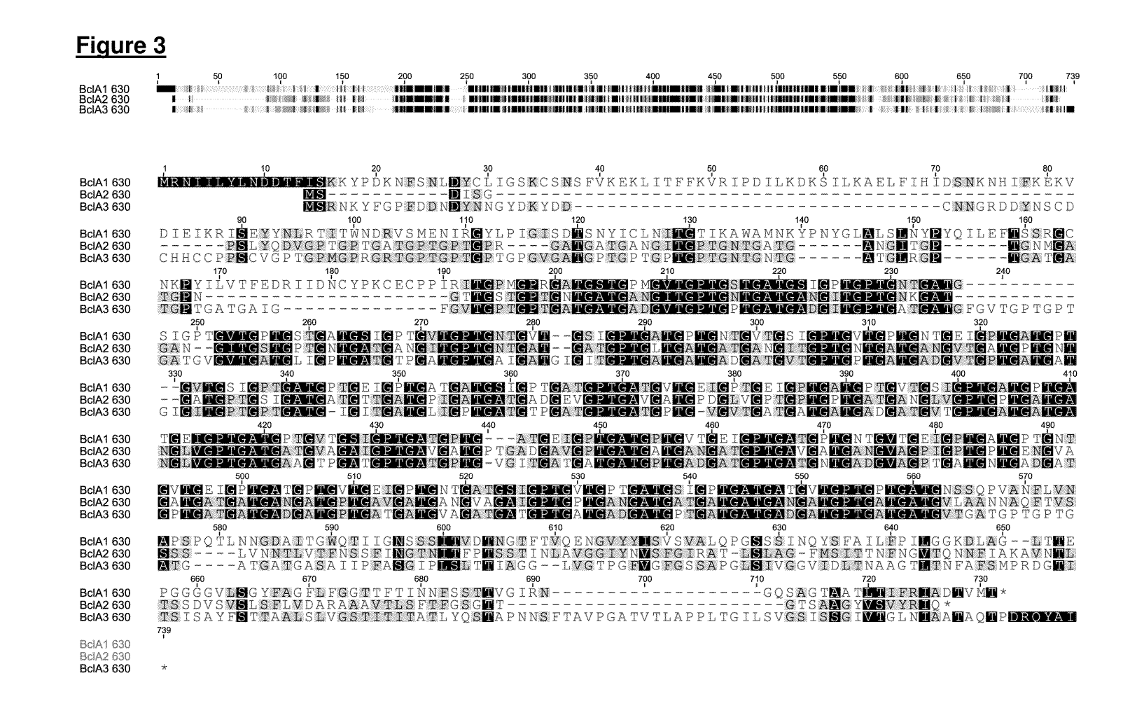

FIG. 2 is a schematic representation of the domain structure of BclA proteins (A) and a pairwise alignment of the same BclA proteins (B). Panel A, Schematic representation of the domain structure of BclA proteins from B. anthracis Sterne (AY995120.1) and C. difficile 630 (BclA1, CAJ67154.1; BclA2, CAJ70128.1; BclA3, CAJ70248.1). The central, collagen-like region (purple) contains multiple GXX repeats and is flanked by the N-terminal (green/blue) and C-terminal (orange/red) domains. In B. anthracis the C-terminal domain mediates trimerization of the BclA monomers while the N-terminal domain is implicated in anchoring the proteins to the exosporia) basal layer. The function of these domains in C. difficile remains to be confirmed. Panel B, Pairwise alignment of the same BclA protein sequences from B. anthracis Sterne and C. difficile 630. Most sequence similarity is limited to the central, collagen-like region. Key: Black, 100% similarity; dark grey, 80-100%; light grey, 60-80; white, <60%. Pairwise identity was generated using ClustalW and a Biosum62 scoring matrix and between all four proteins was 39.1%;

FIG. 3 is a sequence alignment showing the three C. difficile 630 BclA proteins. bclA1=SEQ ID No:4, bclA2=SEQ ID No:5 and bclA3=SEQ ID No:6;

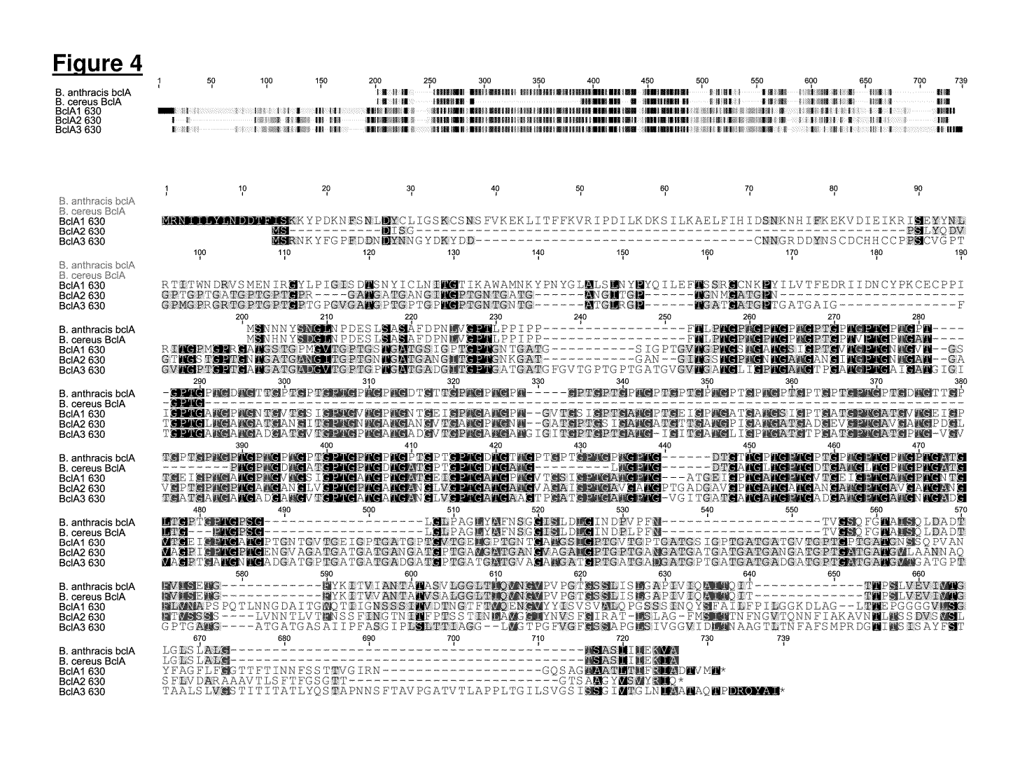

FIG. 4 is a sequence alignment showing the B. anthracis, B. cereus and C. difficile 630 BclA proteins. B. anthracis=SEQ ID No:19, B. cereus=SEQ ID No:20, and bclA/, bclA2 and bclA3 are as shown in FIG. 3;

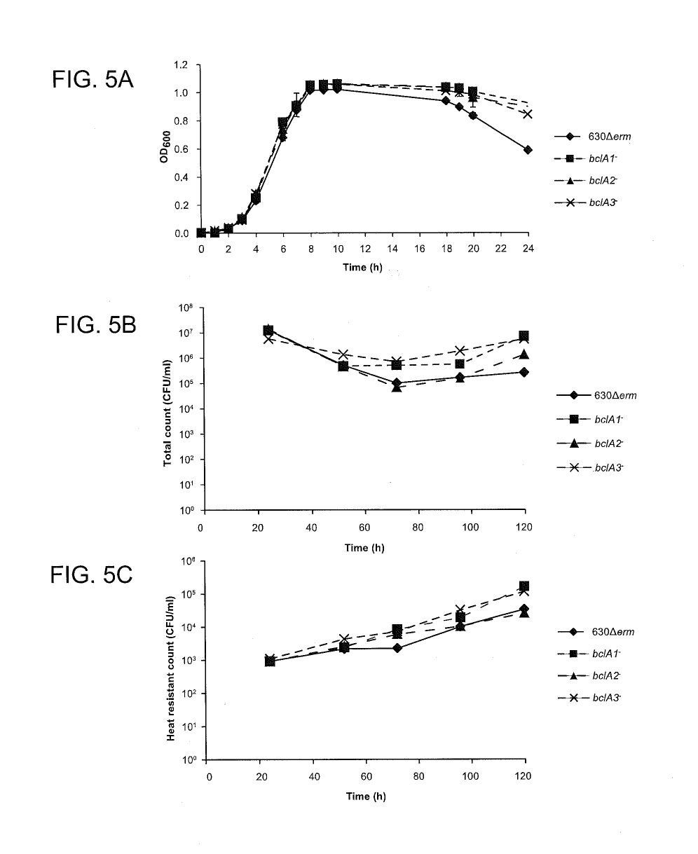

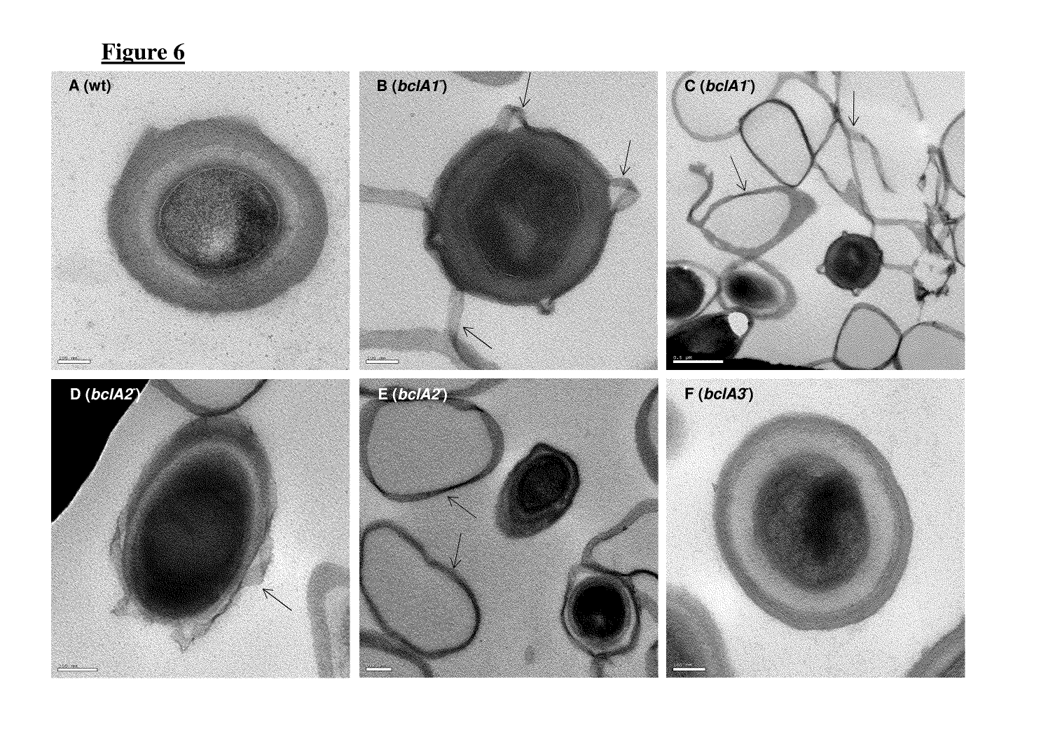

FIGS. 5A-5C show the growth of wild-type and isogenic mutant C. difficile strains grown in BHI medium at 37° C. over 24 h. FIG. 5A) OD₆₀₀, FIG. 5B) Total counts, and FIG. 5C) Heat-resistant (spore) counts. Heat resistance measured at 600 C 20 min;

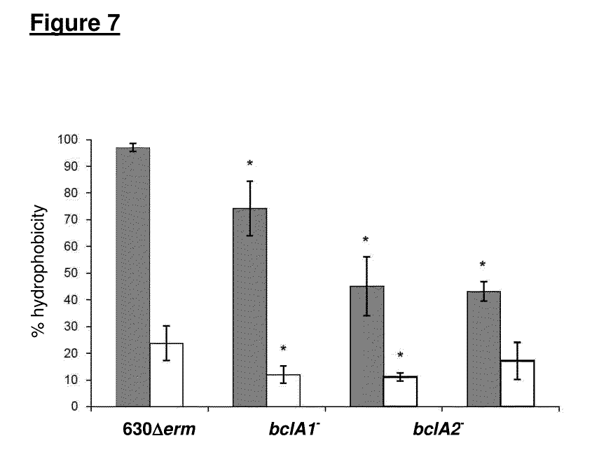

FIG. 6 is an ultrastructure analysis of C. difficile spores by TEM. Panel A, high-magnification image showing purified 630Δerm spores with a normal morphology. Bar: 100 nm. Panels B and C, bclA1⁻ purified mutant spores showing clear defects including a sheet-like material on the outermost layer (arrows indicated). Bars: 100 nm (Panel B) and 0.5 μm (Panel C). Panels D and E, ill-formed bclA2⁻ purified mutant spores with a sheet-like material (arrows indicated). Bars: 200 nm (Panel D) and 0.2 μm (Panel E). Panel F, a bclA3⁻ mutant spore showing normal morphology. Bar: 100 nm;

FIG. 7 is a graph that shows the spore hydrophobicity of wild type (630) and isogenic BclA mutant spores. The SATH assay was used to calculate % hydrophobicity of Histodenz-purified spores of wild-type and mutant spores with (open column) or without sonication (grey column). The analysis was performed three times. * indicates values significantly different between bclA mutants and 630Δerm (bclA1⁻, 0.036; bclA2⁻, 0.0064; bclA3⁻, 0.0006) and sonicated mutant and 630Δerm spores (bclA1⁻, 0.02; bclA2⁻, 0.0243);

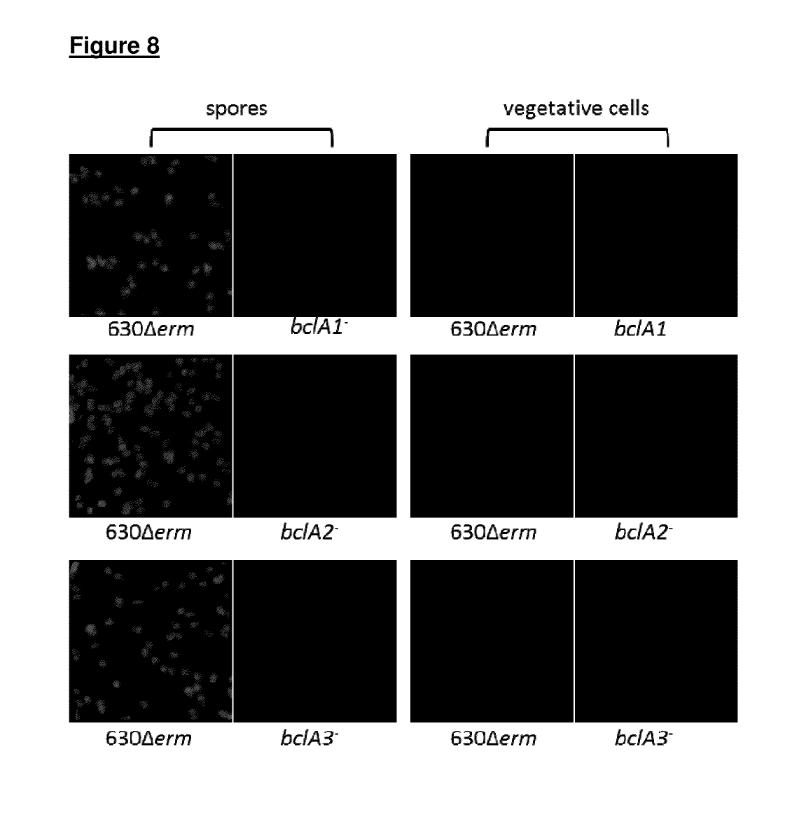

FIG. 8 shows surface display of BclA1, BclA2 and BclA3 using immunofluorescence imaging of suspensions of 630Δerm, bclA1⁻, bclA2⁻ and bclA3⁻ spores (7-day old cultures grown on solid medium) labeled with mouse serum (1:1,000 dilution) raised against each of the three BclA proteins. An anti-mouse IgG-TRITC conjugate was used for secondary labeling. BclA1, BclA2 and BclA3 proteins were detected on both purified and non-purified 630Δerm spores whereas the bclA mutants showed negative signals. Controls included vegetative cells of wild type and mutants;

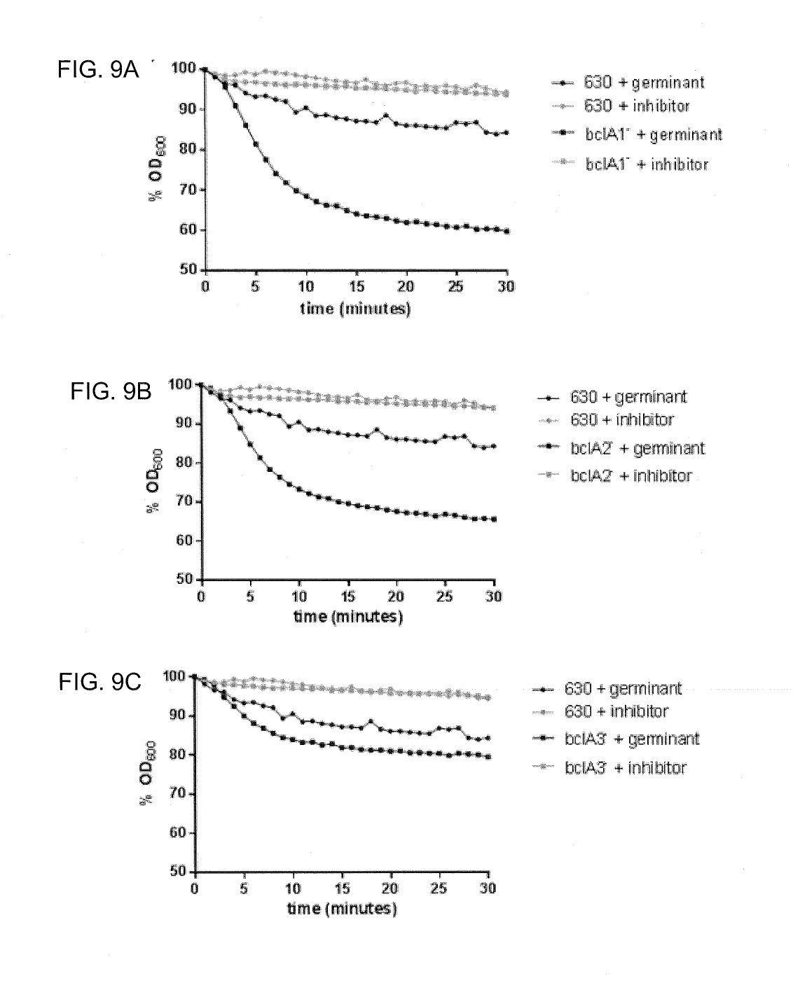

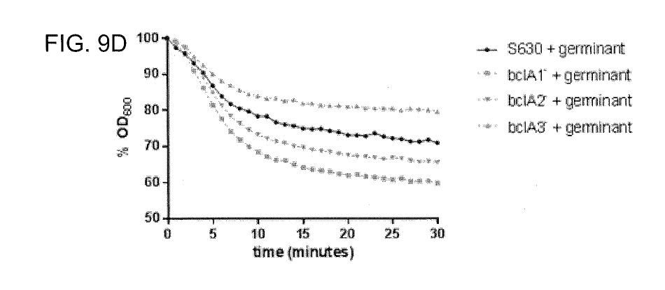

FIGS. 9A-D are graphs showing germination of bclA mutant spores. Germination of spores in BHI media, with either 0.1% sodium chenodeoxycholate (germination inhibitor) or 0.1% sodium taurocholate (germinant). FIGS. 5A-C shows germination of bclA⁻ mutant spores compared with WT 630Δerm spores. FIG. 5D shows germination of bclA⁻ mutant spores compared to sonicated (S) WT spores. Loss of OD600 from starting OD (100%) represents germination of spores as phase brightness is lost at the start of the germination process. % germination was determined as recorded OD600 at time interval/initial OD600)×100;

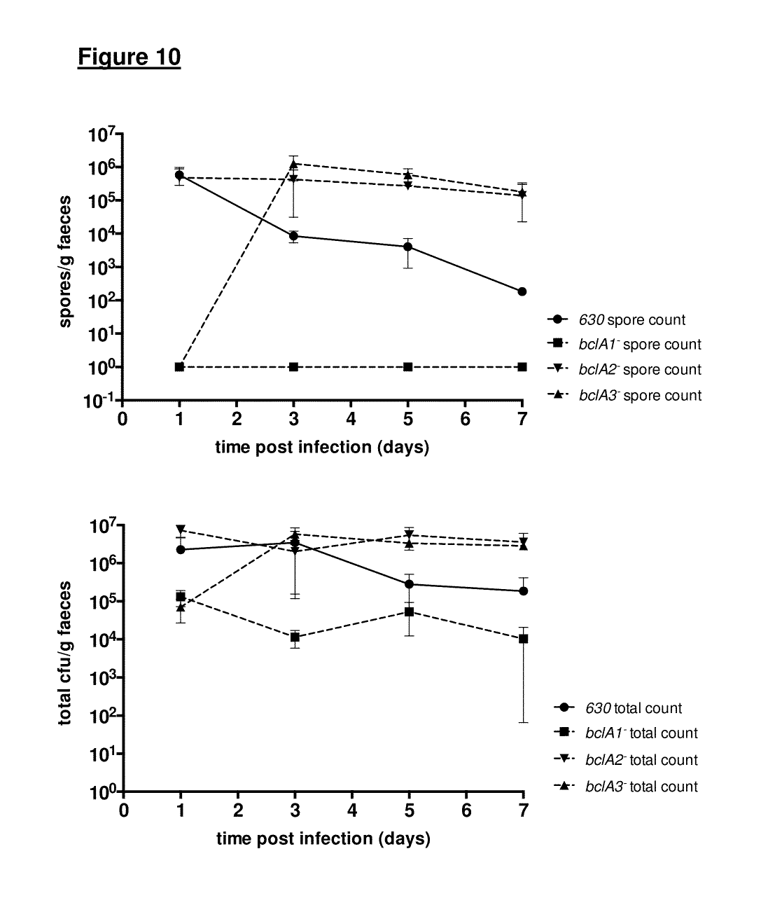

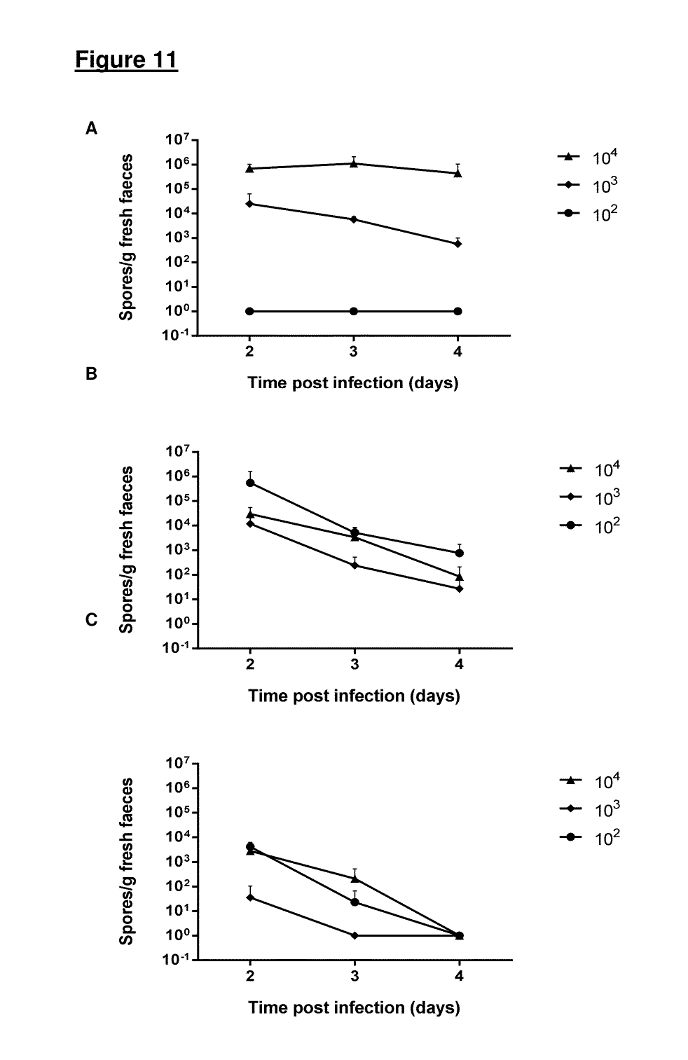

FIG. 10 shows colonization of mice with C. difficile bclA mutants. Panels A-B: Groups of mice (n=4) were orally administered a regimen of cefoperazone and then infected orally with a single dose (1×104) of 630Δerm spores or spores of one of the three bclA mutants. Freshly voided feces was analyzed for heat-resistant spore counts (panel A) and total counts (spores plus vegetative cells) (panel B) on days 1, 3, 5 and 7 post-infection;

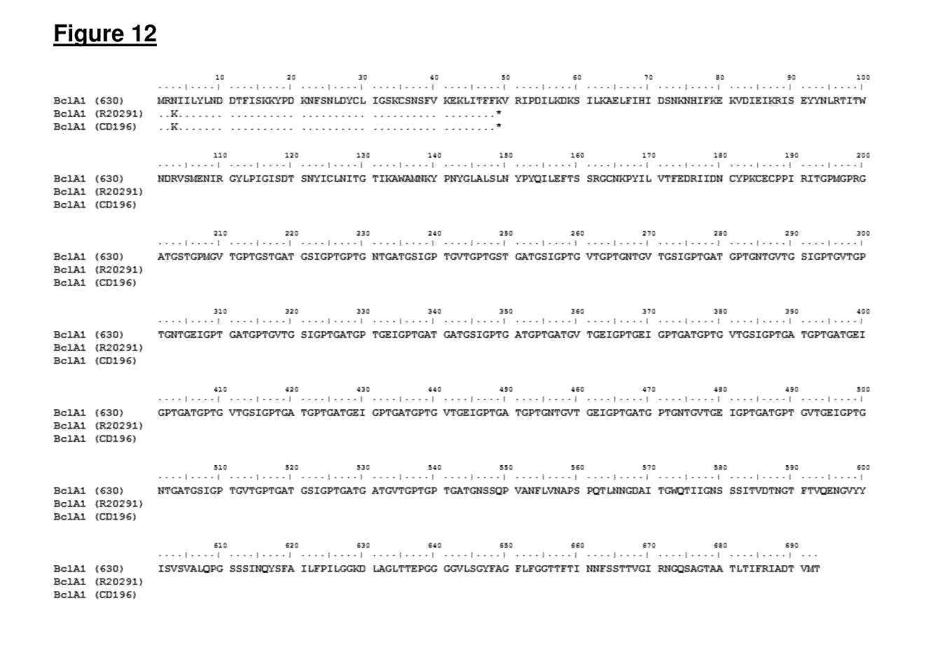

FIG. 11 shows dose-response assays in mice. Mice (n=4) were administered a single dose of clindamycin and five days later infected with R20291 (panel A), 630 Δerm (B) or bclA1− (C) ethanol-resistant spores, at three different dose levels (10², 10³ and 10⁴). Fresh feces was analysed for the presence of ethanol-resistant spore counts following infection. Results are shown as average counts;

FIG. 12 shows BclA1 polypeptides in C. difficile 630, R20291 and CD196 strains. *=stop codon present at position 49 in the bclA1 sequence of the R20291 and CD196 ‘027’ strains (substitution of A145 with T in nucleotide sequence). The R20291 and CD196 sequences are available on the Sanger database. 630 bclA1=SEQ ID No:1, YP 001086801.1 (NCBI), bclA1 R20291=SEQ ID No: 8, CAJ67154.1 (Genbank): and bclA1 CD196=SEQ ID No:21, WP 02138858.1 (NCBI);

FIG. 13 shows expression of BclA proteins in ribotype 027 strains. Immunofluorescence imaging of suspensions of spores of 630Δerm (CD630) and the ribotype 027 strains CD196 and R20291 using antibodies to BclA1, BclA2 and BclA3. In each case spores were prepared in purified and unpurified states from seven day-old plate cultures. Controls included naïve serum and antiserum raised to C. difficile spores and previously described Permpoonpattana, P., E. H. Tolls, R. Nadem, S. Tan, A. Brisson & S. M. Cutting, (2011b) Surface layers of Clostridium difficile endospores. J Bacteriol 193: 6461-6470;

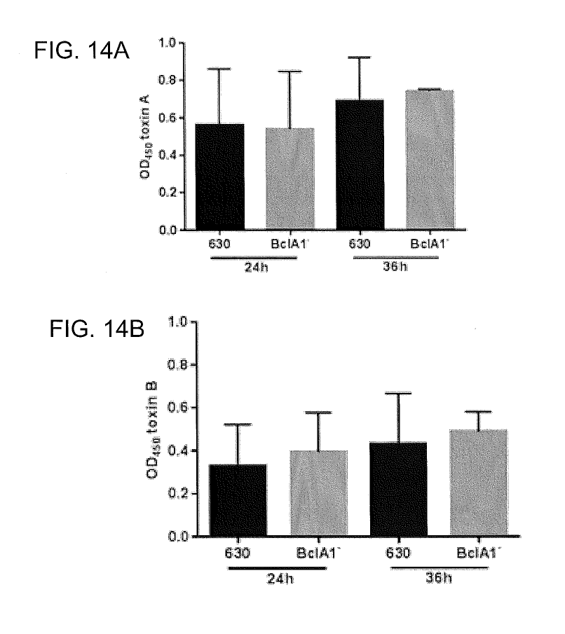

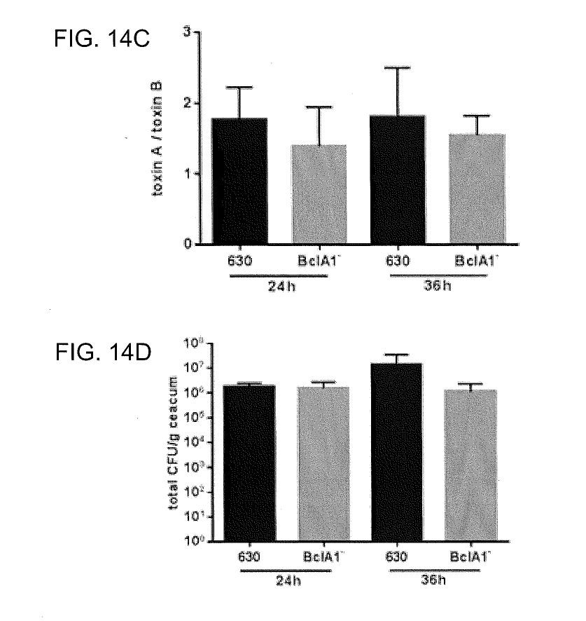

FIGS. 14A-D show Toxins A and B in vivo kinetics; FIGS. 14A and 14B: levels of toxins A (FIG. 14A) and B (FIG. 14B) at different time points (24 or 36 hours) in the caeca of mice infected with 1×105 spores/mouse of the C. difficile 630Δerm wild type strain or bclA1⁻ mutant; FIG. 14C: Ratio between toxin A and toxin B levels in infected mice; FIG. 14D: Total C. difficile CFU counts (cfu/g) in caecum tissues excised from infected mice;

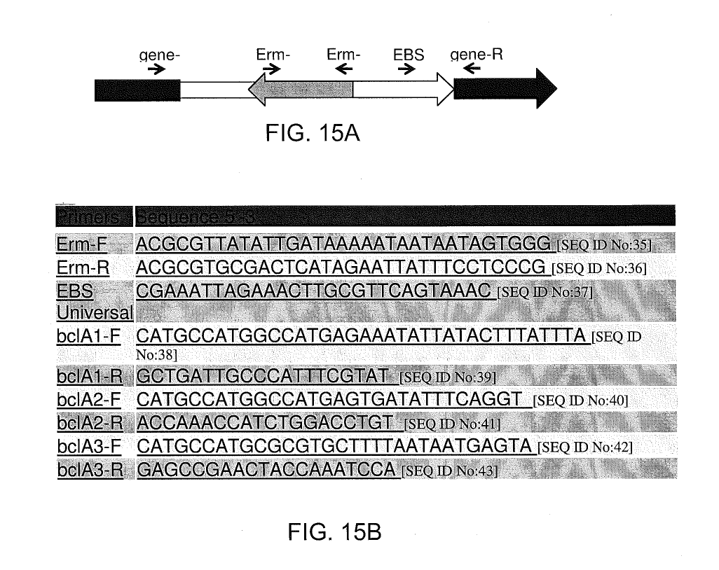

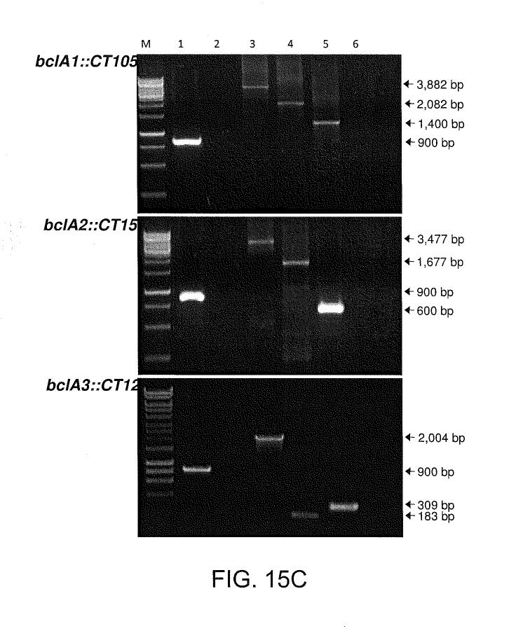

FIGS. 15A-C show inactivation of bclA genes in C. difficile 630 using the ClosTron system. FIG. 15A, gene annotation. FIG. 15B, oligos used for screening mutants by PCR. FIG. 15C, PCR validations of bclA1::CT1050a and bclA2::150a using mutant (odd number lanes) and 630Δerm (even number lanes) genomic DNA amplified with following pairs of primers; lanes 1, 2: ERM-F and ERM-R, lanes 3,4: gene-F and gene-R and lanes 5,6: gene-R and EBS universal. M is a gene marker;

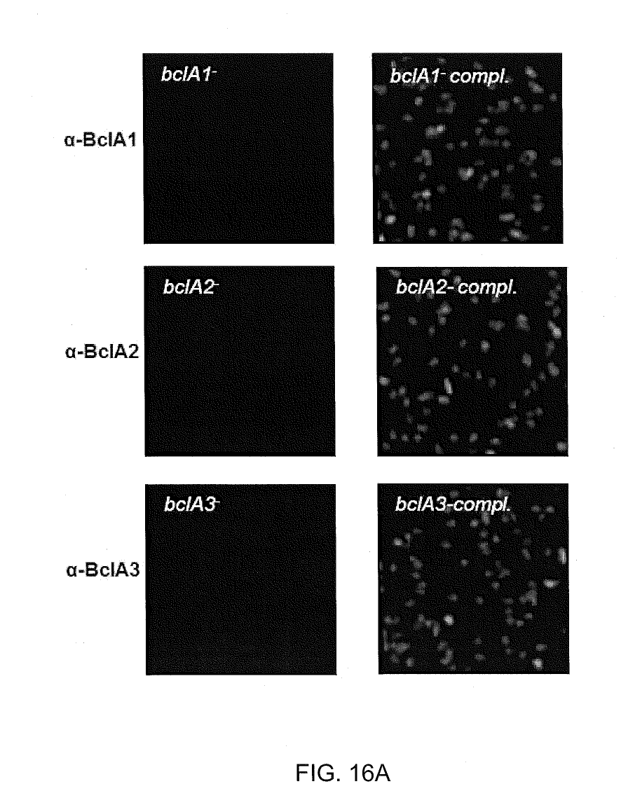

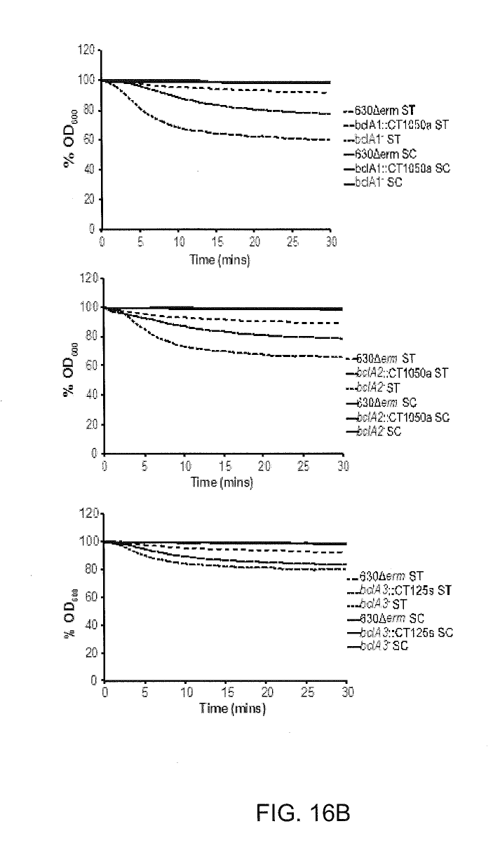

FIGS. 16A-B show complementation analysis of bclA mutants. FIG. 16A pRPF185 plasmids carrying the complete bclA1, bclA2 or bclA3 genes were introduced into the bclA/, bclA2 or bclA3 mutants by conjugation. Spores (purified or unpurified) were prepared and expression of the respective BclA proteins visualised by immunofluorescence microscopy using polyclonal antibodies as shown (right column) and compared to the mutants alone (left column). FIG. 16B Germination studies using Histodenz-purified suspensions of spores (630Δerm), bclA mutants or complemented mutants (::CT105a or CT125s) in the presence of germinant (ST) or inhibitor (SC);

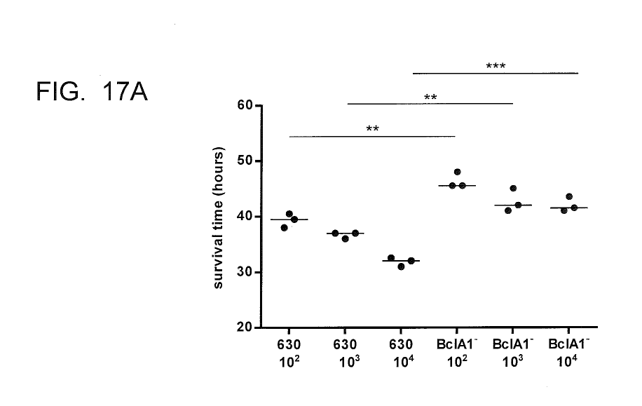

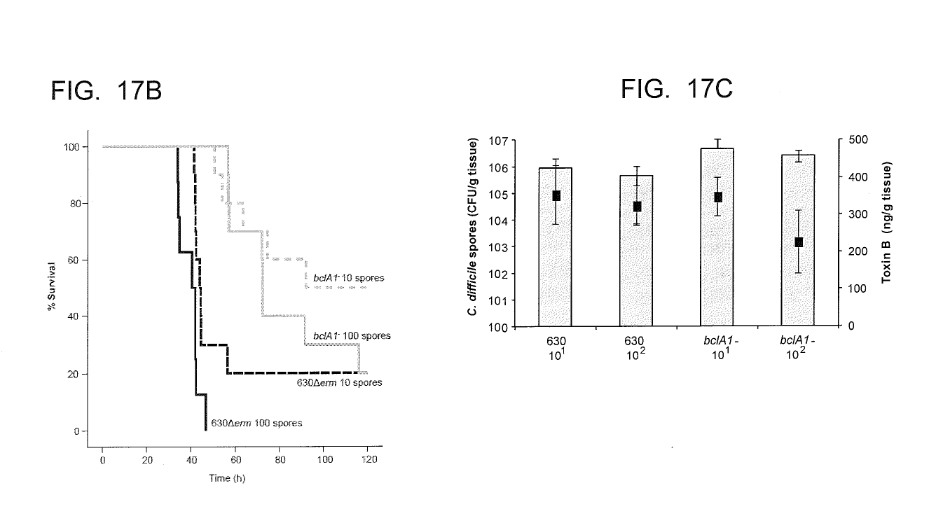

FIGS. 17A-C show infection of hamsters with bclA mutants. The hamster model of infection was used to assess the virulence of the bclA1 mutant. Animals were pre-treated by oral gavage with clindamycin followed by C. difficile spores. FIG. 17A: Survival time for hamsters infected with spores of strain 630Δerm or bclA1⁻. Doses of 10², 10³ or 10⁴ spores were used to infect hamsters (n=3) by oral gavage. 630Δerm black symbols and bclA1⁻ grey symbols. ** p value<0.01; *** p value<0.001. FIG. 17B: Kaplan Meier survival plot of hamsters (n=10) infected with doses of 10 or 10² spores of 630Δerm (black lines) or bclA1⁻ spores. FIG. 17C: Caecum tissue excised from infected hamsters (from FIG. 17A) was evaluated for average counts of ethanol-resistant spores (columns; cfu/g) and toxin B (ng/g; internal black squares). All samples were taken from caecum post-death at the clinical end point of infection;

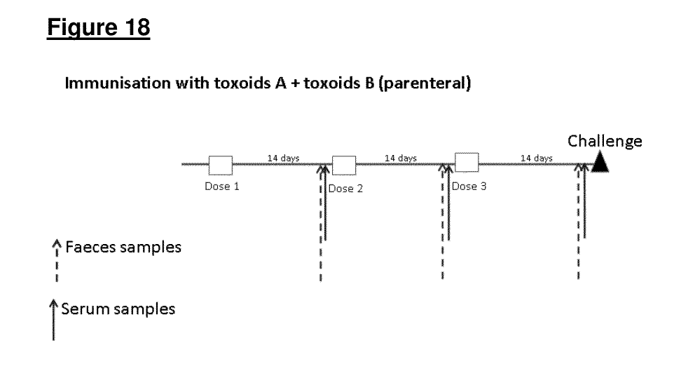

FIG. 18 is an immunisation protocol for toxoids A and B. Toxoids are administered parenterally;

FIG. 19 shows the average spore count of in the cecum and dried feces of mices dosed with toxoids A and B. Experiment repeated 2 times (mouse groups 1(R20291) and 2 (630) with each group being 6 mice). In all cases all mice dosed with toxoids A+B were fully colonised;

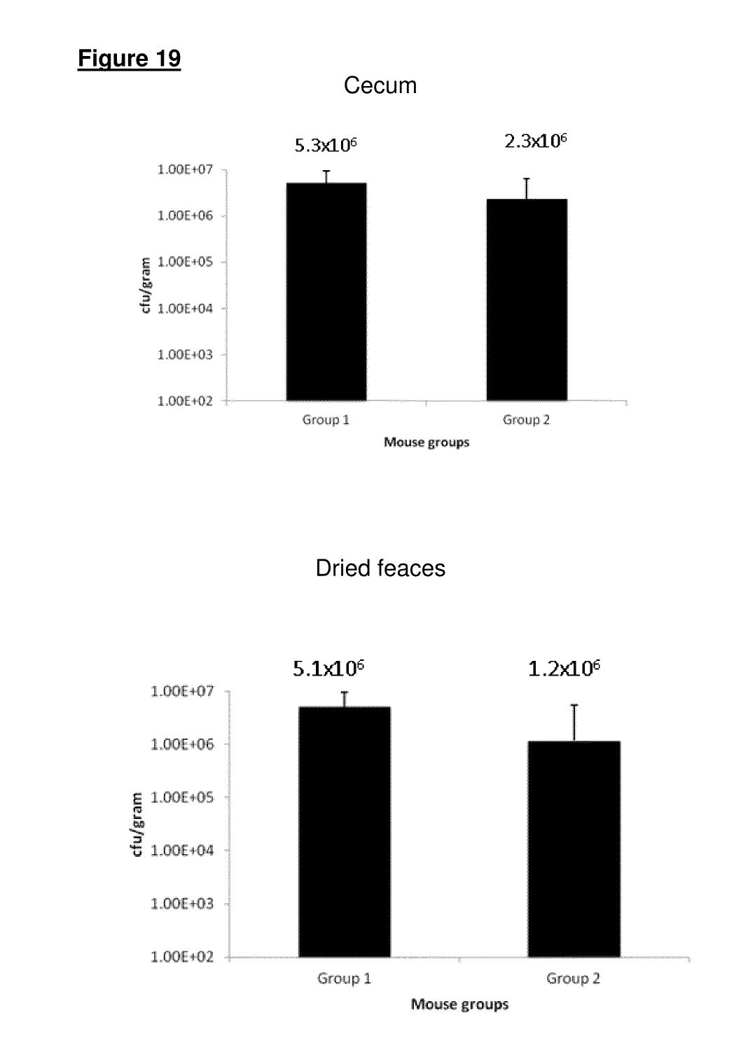

FIG. 20 shows the level of toxin in fresh faeces post infection after injection of toxoids A+B. Average levels of toxin were >2 mg/ml of cecum content for toxA and >0.03 mg/ml toxB;

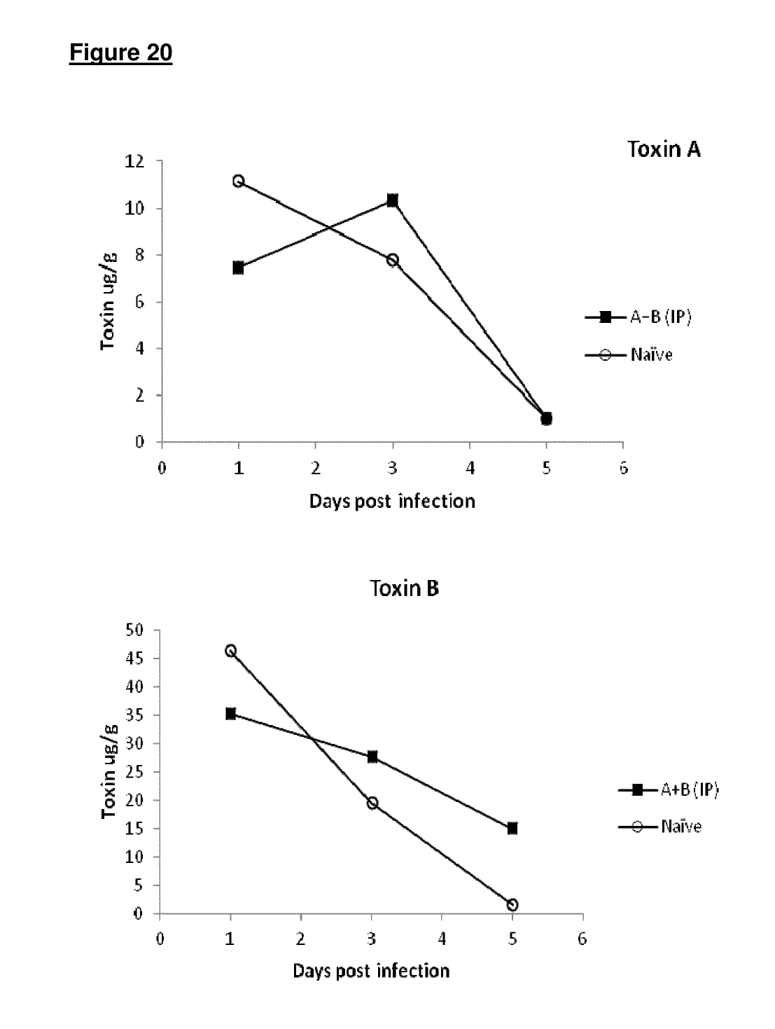

FIG. 21 shows C. difficile colonisation of cecum after oral immunisation with spores of PP108 (CotB-TcdA26-39 CotC-TcdA26-39), PP108+LS1 (CotB-BclA1) of LS1 alone. In each case 5×10¹⁰ spores were given orally to mice per dose. 4 doses were used. Spores CFU were determined in cecum treated with ethanol (20 minutes) to kill vegetative cells. The other 2 groups shown are not relevant.

Viable count=cfu/ml of cecum content;

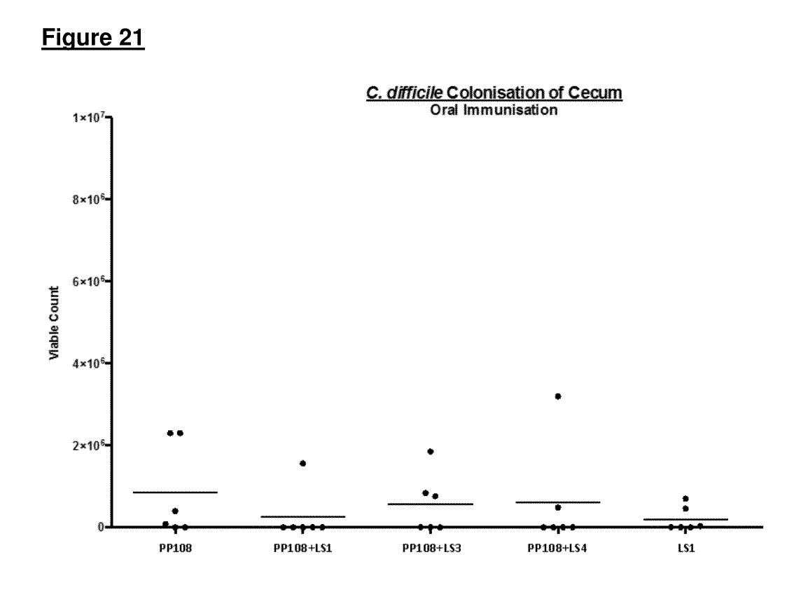

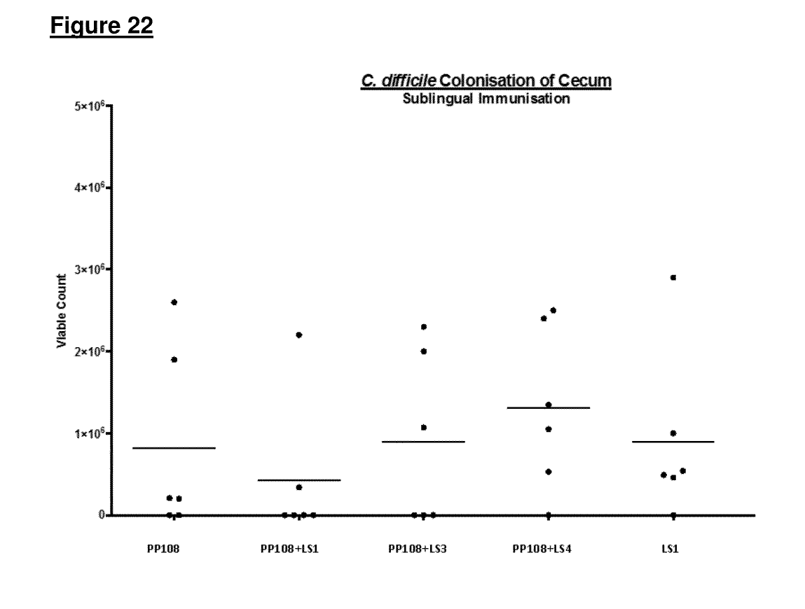

FIG. 22 shows C. difficile colonisation of cecum after sublingual immunisation spores of PP108 (CotB-TcdA26-39 CotC-TcdA26-39), PP108+LS1 (CotB-BclA1) of LS1 alone. In each case 2×10² spores were given sublingually to mice per dose. 4 doses were used. Spores CFU were determined in cecum treated with ethanol (20 minutes) to kill vegetative cells. The other 2 groups shown are not relevant. Viable count=cfu/ml of cecum content;

FIG. 23 shows the levels of toxin A in cecum after oral immunisation with spores of PP108 (CotB-TcdA26-39 CotC-TcdA26-39), PP108+LS1 (CotB-BclA1) of LS1 alone. In each case 5×10⁵ spores were given orally to mice per dose. 4 doses were used. Spores CFU were determined in cecum treated with ethanol (20 minutes) to kill vegetative cells. The other 2 groups shown are not relevant. Naïve are animals dosed with PBC buffer animals;

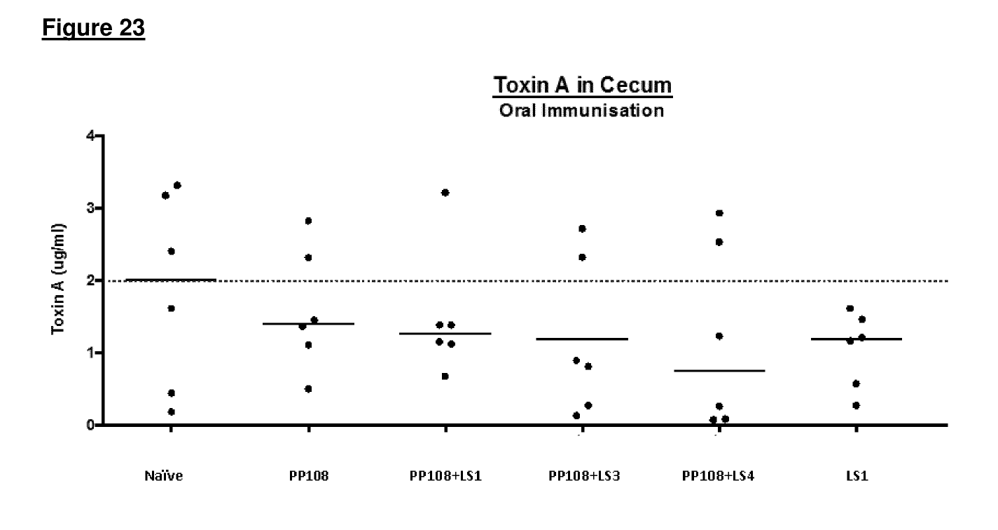

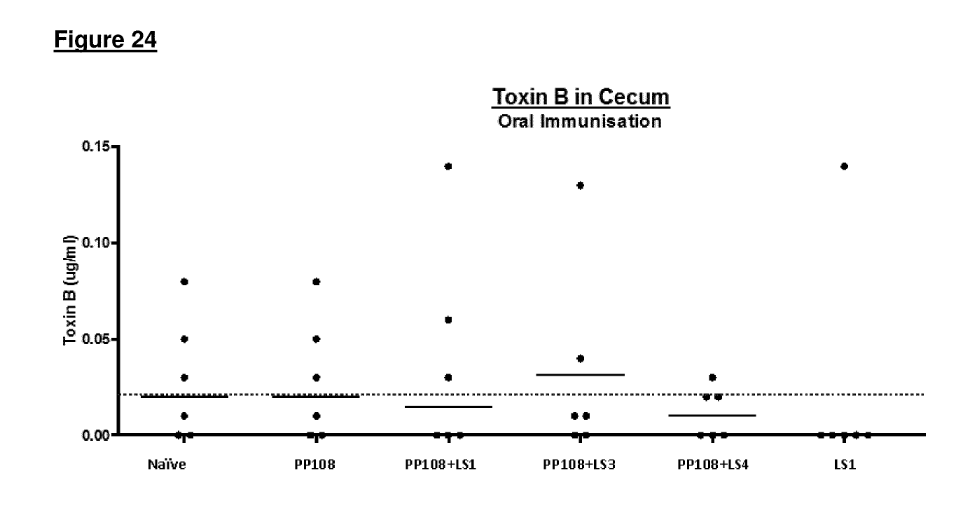

FIG. 24 shows the levels of toxin B in cecum after oral immunisation with spores of PP108 (CotB-TcdA26-39 CotC-TcdA26-39), PP108+LS1 (CotB-BclA1) of LS1 alone. In each case 5×10⁵ spores were given orally to mice per dose. 4 doses were used. Spores CFU were determined in cecum treated with ethanol (20 minutes) to kill vegetative cells. The other 2 groups shown are not relevant;

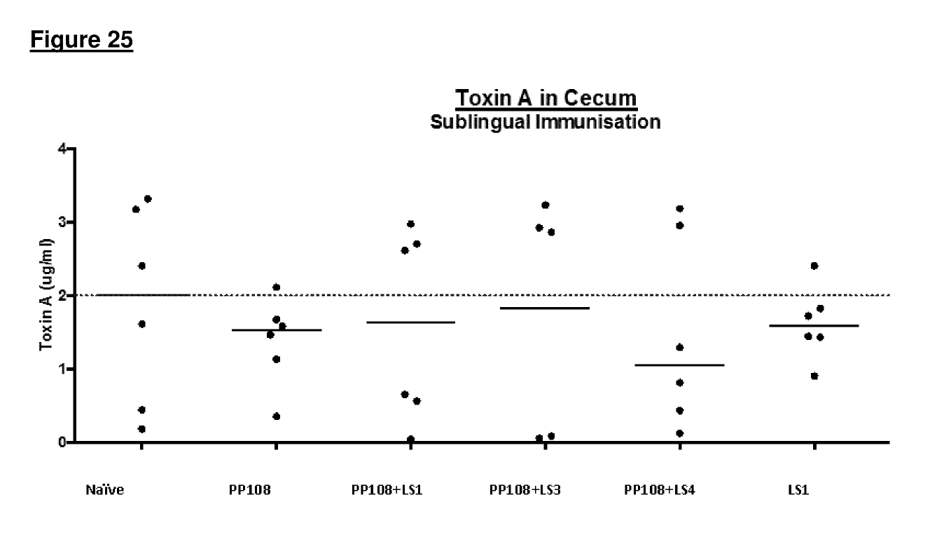

FIG. 25 shows the levels of toxin A in cecum after sublingual immunisation with spores of PP108 (CotB-TcdA26-39 CotC-TcdA26-39), PP108+LS1 (CotB-BclA1) of LS1 alone. In each case 2×10⁵ spores were given orally to mice per dose. 4 doses were used. Spores CFU were determined in cecum treated with ethanol (20 minutes) to kill vegetative cells. The other 2 groups shown are not relevant;

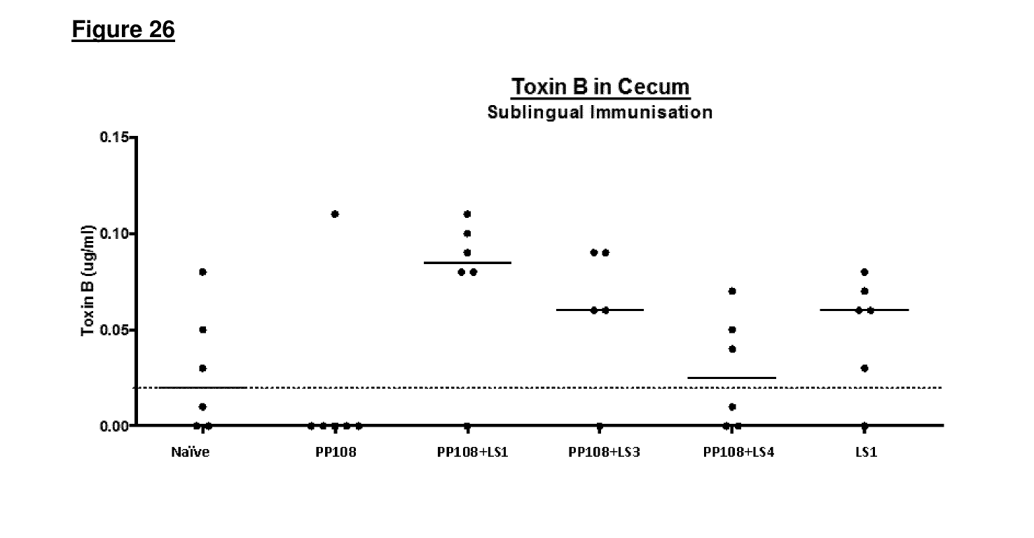

FIG. 26 shows the levels of toxin B in cecum after sublingual immunisation with spores of PP108 (CotB-TcdA26-39 CotC-TcdA26-39), PP108+LS1 (CotB-BclA1) of LS1 alone. In each case 2×10⁵ spores were given orally to mice per dose. 4 doses were used. Spores CFU were determined in cecum treated with ethanol (20 minutes) to kill vegetative cells. The other 2 groups shown are not relevant;

FIG. 27 shows schematic drawings of plasmids (pTS16 and pTS20) used to create fusion proteins using CotB as a carrier; and the 48 amino N-terminal amino acids of BclA1 from 630;

FIG. 28 shows schematic drawings of plasmids (pTS17 and pTS19) used to create fusion proteins using CotC as a carrier;

FIG. 29 shows expression of chimeric proteins on B. subtilis spores using CotB as a carrier protein; and

FIG. 30 shows expression of chimeric proteins on B. subtilis spores using CotC as a carrier protein

### RESULTS

The C. difficile bclA Genes

Three genes encoding BclA-like proteins, annotated as bclA1, bclA2 and bclA3 are present on the genome of strain 630 (FIG. 1) and encode proteins with predicted masses of 67.8, 49.0 and 58.2 kDa respectively. Similar to the BclA proteins found in B. anthracis and B. cereus, all three C. difficile BclA proteins consist of an extensive, central, collagen-like region with multiple GXX repeats flanked by N- and C-terminal domains of variable length (FIG. 2). Most of the triplet repeats are GPT with nearly all containing a threonine residue, that could provide multiple potential sites for O-glycosylation as seen in B. anthracis (Daubenspeck et al., 2004) (FIG. 3). Bioinformatic comparison of BclA protein sequences from B. anthracis, B. cereus and C. difficile revealed that most of the similarity between the proteins is limited to the collagen-like central region since both the C- and N-terminal domains of the C. difficile BclA proteins seem to be distinct from those found in other species (FIG. 4). In B. anthracis BclA these terminal regions are implicated in trimerization of the BclA monomers and their attachment to the exosporia) basal layer (Thompson & Stewart, 2008, Boydston et al., 2005). Notably, the C. difficile BclA proteins do not appear to carry at their N-termini a sequence resembling the motif (LIGPTLPPIPP) that targets the BclA and BclB proteins of B. anthracis to the exosporium (Thompson et al., 2011a, Tan & Turnbough, 2010, Thompson et al., 2011b).

Phenotypes of bclA Mutant Spores

ClosTron mutagenesis can be used to inactivate genes by using a group II intron to insert an erythromycin resistance allele within a target gene (Heap et al., 2009). Using this technique, the three bclA genes were inactivated in strain 630Δerm creating the mutants bclA1⁻, bclA2⁻ and bclA3⁻ (Table 1).

TABLE 1
C. difficile bclA genes and mutations

| | Locus | Encoded | | |
|---|---|---|---|---|
| Gene | Tag¹ | Protein² | Mutant allele³ | Retargeted sequence⁴ |
| bclA1 | CD0332 | Putative | bclA1::CT1050a | ACTCCTGTCGCTCCTGTT |
| | | exosporium | | GGACCTGTTGCT<intron>C |
| | | glycoprotein | | CTGTTGGTCCTATA [SEQ |
| | | (83 kDa) | | ID No: 22] |
| bclA2 | CD3230 | Putative | bclA2::CT150a | GCTCCATTTGCTCCTGTTG |
| | | exosporium | | CTCCTGTCGCC<intron>CC |
| | | glycoprotein | | TGTTGCTCCTGTC [SEQ ID |
| | | (67 kDa) | | No: 23] |
| bclA3 | CD3349 | Putative | bclA3::CT125s | GTCGTGATGATTATAATA |
| | | exosporium | | GCTGTGATTGC<intron>CA |
| | | glycoprotein | | TCATTGCTGTCCAC [SEQ |
| | | (79 kDa) | | ID No: 24] |

¹as described in Sebaihia et al., 2006 and schematically in FIG. 1.
²predicted MW of full-length protein in brackets.
³The mutant allele is shown with a)CT designing ClosTron insertion, b) the number showing the bp within the ORF immediately preceding the ClosTron insertion and c) letter a indicating insertion in the antisense strand.
⁴The 45-bp targeting sequence produced using the www.clostron.com aligorithm and used for mutant construction. The intron insertion site within the 45-mer target sequence is shown.

In bclA1 and bclA2, the erythromycin was inserted in the anti-sense direction, while in bclA3, it was in the sense direction. Mutants were examined for their sporulation and germination phenotypes in parallel with the isogenic Spo⁺ parent strain, 630Δerm. Growth and sporulation of mutants in liquid medium was essentially identical between strains with approximately 10⁴-10⁵ spores/ml produced after five days (FIG. 5). Histodenz-purified spores of all mutants showed no susceptibility to treatment with heat, ethanol and lysozyme (Table 2).

TABLE 2
Resistance properties of bclA mutants¹

| | Untreated | ±SD | Heat | ±SD | Ethanol | ±SD | Lysozyme | ±SD |
|---|---|---|---|---|---|---|---|---|
| 630Δerm | 1.08 × 10⁷ | 1.41 × 10⁶ | 1.46 × 10⁷ | 2.03 × 10⁶ | 5.20 × 10⁶ | 1.05 × 10⁶ | 2.27 × 10⁶ | 7.23 × 10⁵ |
| bclA1⁻ | 1.03 × 10⁷ | 1.77 × 10⁶ | 3.90 × 10⁶ | 4.58 × 10⁵ | 2.87 × 10⁶ | 1.01 × 10⁶ | 4.80 × 10⁶ | 6.08 × 10⁵ |
| bclA2⁻ | 9.20 × 10⁶ | 1.21 × 10⁶ | 4.40 × 10⁶ | 6.24 × 10⁵ | 3.07 × 10⁶ | 4.04 × 10⁵ | 2.23 × 10⁶ | 1.18 × 10⁶ |
| bclA3⁻ | 1.00 × 10⁷ | 1.57 × 10⁶ | 4.20 × 10⁶ | 5.57 × 10⁵ | 2.13 × 10⁶ | 3.51 × 10⁵ | 4.17 × 10⁶ | 9.61 × 10⁵ |

¹Spores purified using Histodenz (Sigma) were tested for resistance to heat, ethanol and lysozyme.
Heat: 10⁷ spores suspended in sterile water were incubated at 60° C. for 24 h.
Ethanol: 10⁷ spores were suspended in 70% ethanol and incubated at RT with agitation for 24 h. After incubation period, spores were washed once with sterile water.
Lysozyme: 10⁷ spores were suspended in a buffer (20 mM Tris HCl (pH 8.0) and 300 mM NaCl) containing lysozyme (1 mg/ml) and incubated with agitation at 37° C. for 20 min.
Serial dilutions for enumeration of surviving spores were plated on BHI agar supplemented with 0.1% sodium taurocholate. Plates were incubated in anaerobic conditions at 37° C. for 48 h.

Transmission electron microscopy (TEM) was used to examine the structure of wild-type and mutant spores (FIG. 6). Compared to 630 Δerm spores (FIG. 6) it was clear that for the bclA1⁻ and bclA2⁻ mutants they carried substantial aberrations in the spore coats. In both cases sheets of coat-like material were present in the medium (FIGS. 6C and 6E) as well as angular projections of material at the spore surface (FIGS. 6B and 6D). The bclA3⁻mutant did not present any apparent defect compared to wild-type spores nor was any coat-like material shed into the culture medium (FIG. 6F). The hydrophobicity of spores was assessed by measurement of the optical density of the aqueous layer after mixing with hexadecane. All three bclA mutants were found to be significantly (P<0.035) less hydrophobic than spores of the wild type 630Δerm (FIG. 7). It has been shown recently that sonication of C. difficile spores is efficient at removing the putative exosporium (Escobar-Cortes et al., 2013) and for comparison we demonstrated here that sonication of spores significantly (P<0.024) reduced the surface hydrophobicity of 630Δerm, bclA1 and bclA2 mutant spores. Polyclonal antibodies raised against recombinant BclA1, BclA2 and BclA3 proteins were used to confirm that each protein was i) located on the surface of 630Δerm spores, ii) absent in vegetative cells and iii) not present in spores of the corresponding isogenic mutant (FIG. 8).

Purified spores were assessed for their ability to germinate in BHI medium supplemented with 0.1% sodium taurocholate as a germinant (Table 3 and FIG. 9).

TABLE 3
Germination phenotypes

% germination^{a}

| Spores | +inhibitor | +germinant |
|---|---|---|
| 630Δerm | 5.6 ± 3.3 | 15.6 ± 4.3 |
| bclA1⁻ | 6.2 ± 2.2 | 40.2 ± 0.7 |
| bclA2⁻ | 5.9 ± 2.0 | 34.4 ± 1.3 |
| bclA3⁻ | 5.3 ± 2.5 | 20.5 ± 1.8 |
| 630Δerm | 5.6 ± 0.6 | 29.1 ± 0.9 |
| (sonicated) | | |

^{a}% germination was determined as % loss in OD₆₀₀ in the presence of inhibitor (0.1% sodium chenodeoxycholate) or germinant (0.1% sodium taurocholate).

Germination correlates to a loss in OD₆₀₀ as spores rehydrate and become phase dark. 630Δerm spores germinated relatively slowly with a 16% reduction in OD₆₀₀ over a 30-minute period. All three mutants germinated faster than the wild-type strain with the bclA1⁻ and bclA2⁻ mutants exhibiting the highest germination rates with 40% and 34% loss in OD₆₀₀ respectively, over 30 min. As a control, spore germination was conducted in parallel in the presence of the inhibitor sodium chenodeoxycholate. In the presence of this inhibitor, spores of wild-type and all bclA mutant strains remained stable and exhibited a maximum OD₆₀₀ drop of 5-6% over 30 minutes. Germination of sonicated spores of the wild-type strain, evaluated in parallel, revealed an OD₆₀₀ drop of 29%, indicating that disruption of the spore surface layers enhanced germination (FIG. 9).

Infectivity of bclA Mutants in the Mouse Model of Infection

The recently described mouse model of cefoperazone pre-treatment to induce C. difficile infection (Theriot et al., 2011) was used to evaluate the progress of shedding of C. difficile spores. Animals were given a single dose (10⁴) of mutant or wild-type spores (FIG. 10). Total counts (spores and vegetative cells) of C. difficile shed in the feces ranged from 10⁴ to 10⁶ per gram although somewhat lower counts were found for the bclA1⁻ mutant (FIG. 10B). Mice body weights remained similar with no significant differences between groups (data not shown). Heat resistant spore counts of 630Δerm-dosed mice declined over time (FIG. 10A). Spores were not found in the feces from mice dosed with the bclA3⁻ mutant on day 1, even if substantial levels of spores were detected on days 3, 5 and 7. Spore counts of both the bclA2⁻ and bclA3⁻ mutants increased after days and were substantially higher (>1-log) on days 3, 5 and 7 if compared to that of wild-type infected animals (FIG. 10A). Surprisingly no heat resistant spores were detected for the bCLA1⁻ mutant in the feces post-infection (FIG. 10A) in all the time points. The experiment has been repeated with similar findings. However, using a dose-response assay (Table 4 and FIG. 11) where counts were detected following ethanol treatment spores of the bclA1⁻ mutant were clearly detected in the feces albeit at lower levels than in mice infected with wild type 630 spores.

TABLE 4
Infectivity of spores of different C. difficile strains in mice^{a}

| Strain | ID₅₀^{b} |
|---|---|
| 630Δerm | 1 × 10² |
| R20291 | 1 × 10³ |
| bclA1⁻ | >1 × 10⁴ |

^{a}Groups of mice were first treated with clindamycin followed by a 5-day interval before being given three doses (10², 10³ or 10⁴) of spores followed by determination of ethanol-resistant spores counted in fresh fecal samples (cfu data is shown in Sup. F6). Colonization was defined as animals carrying >10³ spores/g feces at 48 h post-infection. Using the Reed-Munch equation (Ozanne, 1984) the dose of spores required to infect 50% of mice (ID₅₀) was determined.

This suggested that the bclA1⁻ mutant spores in fecal samples were susceptible to heat treatment but not to ethanol. One explanation might be that the bclA1⁻ mutant, being more germination proficient than the isogenic parent strain 630 was more susceptible to heat treatment, or more likely, that heat was producing premature germination of bclA1⁻ mutant spores. For this reason, in subsequent analysis we used ethanol for measurement of wild type and mutant spores. Returning to the dose response assay this showed that the number of spores required to infect 50% of mice (ID₅₀) was 2 logs higher in the bclA1⁻ mutant compared to the wild-type control. In contrast to 630, spores of the bclA1⁻ mutant were not detectable three and four days post infection (FIG. 11). Together these data show that bclA1⁻ mutants are less infective than wild-type strains.

### Reduced Colonization by a ‘Hypervirulent’ 027 Strain

Analysis of the bclA1 genes in the genome sequences of two ribotype 027 strains, R20291 and CD196 (Stabler et al., 2009) revealed a stop codon at position 48 in addition to an asparagine to lysine change at position 3 in the ORF (FIG. 12). We have independently sequenced the bclA1gene in R20291 and confirm that the stop codon is present and is not a sequencing error. As such, the 027 strains must each encode a BclA1 protein of approximately 6 kDa and, being significantly smaller than the one found in strain 630, would presumably lack function. Using antibodies raised against BclA1 (from strain 630) we have been able to identify BclA1 on the surface of both R20291 and CD196 spores suggesting that the truncated protein can assemble into the exosporium in these 027 strains (FIG. 13).

R20291 is a so-called ‘hypervirulent’ strain (Stabler et al., 2009, Buckley et al., 2011), is clinically relevant and would, prima facie, be considered more virulent than the 630 strain. Our studies suggest that bclA1 deletion would impair colonization. Therefore, to determine the infectivity of a 027 strain carrying a truncated BclA1 protein, the ability of R20291 spores to colonize mice was analyzed as previously described for 630Δerm and the bclA1 mutant (Table 4 and FIG. 11). The ID₅₀ for R20291 was 1×10³ and therefore ten-times less infectious than 630 but more infectious than a strain completely devoid of BclA1, suggesting a correlation between the presence of an intact BclA1 protein and the susceptibility of mice to colonization. Interestingly, compared to the 630Δerm spores, R20291 spores (i.e., at doses≧10³) were able to persist longer in the GI-tract and were maintained at higher levels.

### BclA1 is a Virulence Determinant in Hamsters

Hamsters provide a more acute model of C. difficile infection (Sambol et al., 2001, Goulding et al., 2009) with wild-type strains causing a rapid fulminant infection most likely due to the sensitivity of these animals to C. difficile toxins. Accordingly, this model was used to evaluate the infectivity of bclA1⁻ mutant spores. In a preliminary study, groups of three hamsters were dosed with 10², 10³ or 10⁴ of 630Δerm or bclA1⁻ spores (FIG. 14). Significant differences were observed in survival times between wild type and mutant (10², P=0.003; 10³, P=0.008; 104, P=0.0003) as well as in the dose-dependent response. Using an infective dose of 102 630Δerm spores the clinical end point was reached in approximately 40 h while this was delayed until approximately 47 h with the same dose of bclA1⁻ mutant spores. This study was repeated using ten hamsters per group and two doses, 10 and 10² spores, of either 630Δerm or the bclA1⁻ mutant. As shown in FIG. 14B, a dose of 102 spores of 630Δerm resulted in no survival of infected animals while a lower dose of 10² spores resulted in the survival of two animals. By contrast, the bclA1⁻ mutant was clearly less infective with 50% survival following a dose of 10 spores and 20% survival using 102 spores. The calculated ID₅₀ for 630Δerm spores was 2.37×101 and for the bclA1⁻ mutant 2×10², indicating that the bclA1⁻ mutant was ten-times less infective. Animals infected with either 630Δerm or bclA1⁻ had similar levels of C. difficile spores in the caecum (FIG. 14C), although levels were somewhat higher in bclA1⁻ infected animals, possibly reflecting the ability of this mutant to germinate more efficiently and proliferate. The levels of toxin B in caecum samples were measured and found to be similar in all samples, showing no significant differences (FIG. 14C). In surviving animals no viable C. difficile or toxin B could be detected in caeca. This data supports the murine study demonstrating that bclA1⁻ mutant strains, although able to produce toxins, are clearly less infectious than the wild-type.

It was possible that the low infectivity of the bclA1⁻ mutant might have arisen if toxin production was reduced or delayed in vivo. This is unlikely though since based on the morphogenesis of the spore, we would predict that the bclA1 gene would be expressed in the late phase of spore formation, while toxin production is associated with the stationary phase of vegetative cell growth (Rupnik et al., 2009) and should occur before bclA1 expression. Preliminary qPCR data (not shown) demonstrated that tcdA and tcdB are expressed during stationary phase and the early stages of spore formation, while the bclA1-3 genes are expressed at the terminal stages of sporulation (approx. 9 h following the onset of development). To rule out differences in production of toxins in vivo between 630Δerm and the bclA1 mutant, we infected mice eight days post clindamycin treatment with a high dose (10⁵/mouse) of 630Δerm or bclA1⁻ spores sufficient to cause infection in most of the mice (see Table 4). At 24 and 36 hours post infection the total CFU of C. difficile and toxin A and B levels were determined in caeca. As shown in FIG. 14, the total CFU in mice infected with 630Δerm or bclA1⁻ spores were equivalent at both time points and no differences were observed between toxin A and B levels in the caeca and in the ratio between the two toxins.

### Use of Fusion Genes/Proteins

FIG. 27 shows schematic drawings of plasmids (pTS16 and pTS20) used to create fusion proteins using CotB as a carrier, and FIG. 28 shows schematic drawings of plasmids (pTS17, and pTS19) used to create fusion proteins using CotC as a carrier. These plasmids were used to express chimeras of BclA1 on the surface of B. subtilis spores, as shown in FIGS. 29 and 30.

### Mucosal Vaccinations

Parenteral dosing of toxoids A+B (FIG. 18) using 3 doses showed no effect on colonization with C. difficile strains 630 or R20291 (FIG. 19). Toxin levels were unaffected and no different from naïve mice in animals dosed intra-peritoneal with toxoid A+ toxoid B. Toxins were measured in faeces (FIG. 20) as well as in cecum.

In the mouse model, a combination of spores expressing CDTA14, CotE and BclA1 were evaluated.

### Strains

PP108=spores expressing CDTA14
LS1=spores expressing an N-terminal fragment of BclA1 (an exosporia) protein)
LS3=spores expressing BclA1-CotE fusion
LS4=spores expressing CotE

Initial results show that all combinations of spores produced a positive effect on colonization and significantly reduced colonization vs injection of toxoids A+B. In some cases no colonization of the cecum was observed. A combination of spores expressing CDTA14 (the C-terminus of toxin A) and either CotE (C-terminus) or BclA1 (N-terminal 48 amino acids) provides better decolonization than CDTA14 spores (PP108) alone.

Oral dosing (FIG. 21) performed better than sublingual (FIG. 22) based on reduction of both toxin A and toxin B levels (FIG. 23-26). Based on this data the inventors predict that CotE spores (LS4) combined with CDTA14 spores (PP108) or PP108 combined with BclA1 spores (LS1) would provide superior levels of protection (compared to PP108 alone) in the hamster model of C. difficile infection.

Finally, as can be seen in FIGS. 29 and 30, BclA and CotE chimeric proteins can be effectively expressed on the surface of B. subtilis spores using B. subtilis CotB and CotC as carrier proteins, respectively.

### Discussion

The exosporium is poorly defined in C. difficile and images of this ‘sac-like’ outer layer vary from a well-defined thick, electron dense laminated structure (Lawley et al., 2009b) to more diffuse layers that are easily removed from the underlying spore coat (Permpoonpattana et al., 2011b, Permpoonpattana et al., 2013, Escobar-Cortes et al., 2013). Most probably the exosporium of C. difficile is particularly fragile at least under the conditions commonly used in the laboratory to prepare spores so defining this structure in C. difficile remains elusive. One of the major immunodominant proteins found in the exosporium of B. anthracis and B. cereus is the BclA protein (Sylvestre et al., 2002, Redmond et al., 2004, Steichen et al., 2003, Todd et al., 2003). Filaments of the BclA protein form the hairy nap which is characteristic of the exosporia of the Bacillus/anthracis/thuringiensis family of spores (Kailas et al., 2011) but in the case of C. difficile these hair-like filaments have yet to be observed. C. difficile carries three bclA genes whose products share similarity with the BclA proteins of B. anthracis and B. cereus. However, the composition of these proteins differ significantly especially with regard to the absence of the N-terminal (targeting the exosporium) and C-terminal (oligomerization) domains. Our evidence suggests that the C. difficile BclA proteins reside in the outermost layers of the spore and most probably the putative exosporium. Antibodies against all three BclA proteins confirmed expression on the spore surface and mutagenesis of the three genes also revealed noticeable defects in the spore coat. First, in two mutants, bclA1 and bclA2, aberrations in the spore coat were clearly evident and presumably assembly of the outer coat or exosporium is defective in these mutants emphasizing that both proteins are likely major exosporia) proteins. Second, spores of all three mutants had significantly reduced hydrophobicity. Reduced hydrophobicity was also apparent in spores that had been sonicated, an approach that has been shown elsewhere to remove the exosporium (Permpoonpattana et al., 2011b, Permpoonpattana et al., 2013, Escobar-Cortes et al., 2013). In B. anthracis, bclA mutants also have a much-reduced hydrophobicity where the exosporium is thought to provide a water repellant shield reducing its ability to interact with the host matrix (Brahmbhatt et al., 2007). Third, all three bclA mutants showed increased germination rates, a characteristic also found in the B. anthracis bclA mutant and presumably a result of a defective exosporium allowing access of germinants to receptors situated in the innermost spore membranes (Brahmbhatt et al., 2007, Carr et al., 2010). Finally, in vivo infection studies in mice revealed that the bclA1 and bclA3 mutants had impaired colonization efficiencies although this was most striking with the bclA1 mutant that completely failed to colonize the mouse GI-tract. Thus, the three BclA proteins are integral components of the outermost layers of the spore (and most probably the exosporium) and whose removal severely destabilizes this outermost layer allowing access of germinants and reducing surface hydrophobicity.

In B. anthracis BclA has not been shown to play a significant role in virulence with a bclA mutant having no effect on pathogenicity in mice or in guinea pigs (Bozue et al., 2007) and with mutant and wild-type strains having similar LD₅₀ values (Brahmbhatt et al., 2007). This is in marked contrast to our study where we show that in C. difficile at least one BclA protein, BclA1, is involved in the initial stages of colonization and infection. In mice and in hamster models of infection spores devoid of BclA1 were up to 2-logs less infective (i.e., by ID₅₀) than isogenic wild-type spores and showed increased times to death in hamsters. This suggests that BclA1 could be involved in the initial stages of host colonization and that this event must be mediated by the spore, an event occurring before spore germination. Even more intriguing was the observation that two 027 strains carried truncated BclA1 proteins and that one of them, R20291, a so-called ‘hypervirulent’ strain, was actually less infective in a mouse model of infection than its counterpart 630 suggesting a relationship of animal susceptibility to the presence of an intact BclA1 protein in the C. difficile spore. Spores of strains carrying a full length BclA1 protein (i.e., 630) were more infectious than those carrying a defective or truncated bclA1 gene. Only 102 spores of 630 were required for 100% colonization in hamsters but using the same dose lower levels of infection were found with a variety of B1 strains (Razaq et al., 2007). Similarly, 104 spores of R20291 have been shown to produce complete infection in hamsters (Buckley et al., 2011). Finally there is now evidence showing that hamsters are more susceptible to colonization with non-toxigenic strains of C. difficile than with toxigenic strains (e.g., M68 and B1-7) (Buckley et al., 2013).

It has been proposed that hypervirulent 027 strains may have acquired additional virulence genes based on the considerable genetic differences between the epidemic and non-epidemic strains (Stabler et al., 2009). However, we suggest that in terms of initial colonization the hypervirulent R20291 strain is actually less effective, that is, animals are less susceptible. This then raises some interesting and provocative questions. We wonder whether animals including humans are actually less susceptible to ‘hypervirulent’ strains yet once colonization occurs the severity of disease is much greater. In many ways this resembles the situation of influenza where seasonal flu strains are typically highly infective but of low severity compared to the low infectivity-high severity nature of H5N1 strains. If what happens in humans mirrors that in mice then the virulence of R20291 must arise not due to its infectivity but rather, due to some other factor affecting the severity of infection, e.g., levels of toxin production, increased persistence or faster germination. For the 027 ‘hypervirulent strains increased toxin production and biofilm formation (Dawson et al., 2012, Dapa & Unnikrishnan, 2013) have been identified as potential virulence factors. However, the presence of an intact BclA1 protein would correlate with the susceptibility of the host to infection and we assume that BclA1 may interact with a specific host target. It is clear that BclA1 plays a key role in the initial stages of infection and host susceptibility. Current thought is that C. difficile is acquired primarily from the environment but is it possible for hypervirulent strains to remain as latent members of the gut flora and to be rendered infectious only after their numbers reach a critical level resulting from antibiotic-disturbance?

In B. anthracis it has recently been shown that BclA interacts with the integrin Mac-1 leading to uptake by professional phagocytes. Rhamnose residues within BclA have been shown to interact directly with CD14 molecules (Oliva et al., 2009). If C. difficile BclA1 also recognizes a specific target then it is a prime candidate for inclusion in a more robust vaccine to C. difficile infection. In preliminary trials we have expressed the 48 amino acid N-terminus of BclA1 on the surface of B. subtilis spores. This segment is that which is present in the 027 strain R20291 (FIG. 12). When combined, 50:50, with B. subtilis recombinant spores expressing the carboxy-terminus of toxin A (strain PP108 as described elsewhere (Permpoonpattana et al., 2011a)) they were able to provide 100% protection when administered orally to mice compared to about 50% protection when immunized with PP108 spores alone (data not shown). This is encouraging and suggests that BclA1 could act as a decolonization factor and could be combined with an anti-toxin based vaccine to prevent C. difficile infection.

In summary, BclA1 (N-terminus) and CotE (C-terminus) are new antigens that confer some level of protection in animal models. They can be delivered mucosally (by oral dosing) using heat stable bacterial spores and provide decolonisation of C. difficile. They would achieve this by inducing mucosal (secretory IgA) responses that prevent spores of C. difficile from colonising the gut epithelium. Antibodies to BclA1 and CotE are therefore protective. We show here that the use of spores displaying BclA1, CotE or BclA1-CotE confers greater levels of protection (using toxin production and colonisation as indicative markers of C. difficile infection) when adminstered in combination with B. subtilis spores expressing a C-terminal fragment of toxin A (TcdA26-39) that use of spores expressing TcdA26=39 alone. Therefore in a vaccine formulation we would consider a combination of spores expressing TcdA26-39 and spores expressing BclA1, CotE or BclA1-CotE.

In the case of BclA1 we have identified the utility of the N-terminal domain as being important for protection. We also show that the BclA1 and CotE domains can be combined as chimeras and be expressed as fusions to two protein components of the Bacillus subtilis spore coat, CotB and CotC. This teaches us that the N-terminal domain of BclA1 and the C-terminal domain of CotE can be stably expressed on the spore surface (fused to spore coat proteins) and be expressed together as a BclA1-CotE chimeras.

Since the N-terminal domain of BclA1 is conserved among all C. difficile strains this region is critical in a vaccine formulation.

### Experimental Procedures

### Strains

630 is a toxigenic (tcdA⁺tcdB⁺) strain of C. difficile isolated from a patient with pseudomembranous colitis during an outbreak of C. difficile infection (CDI) (Wust et al., 1982). For ClosTron mutagenesis and mutant analysis an erythromycin-sensitive derivative 630Δerm (Hussain et al., 2005) was used (provided by N. Minton, Univ. Nottingham, UK). R20291 is an epidemic strain of ribotype 027 isolated from Stoke Mandeville Hospital in 2006 (Stabler et al., 2009) and was obtained from T. Lawley (Wellcome Trust Sanger Institute, UK).

Growth of C. difficile and Preparation of Spores

C. difficile was routinely grown in vegetative culture by overnight growth in TGY-medium (Paredes-Sabja et al., 2008). Spores of C. difficile were prepared by growth on SMC agar plates using an anaerobic incubator (Don Whitley, UK) as described previously (Permpoonpattana et al., 2011a). After growth for seven days at 37° C. spores were harvested and either washed three times with water or purified using HistoDenz as follows. Crude spore suspensions were washed five times with ice-cold sterile water, re-suspended in 500 μl of 20% HistoDenz (Sigma) and layered over 1 ml of 50% HistoDenz in a 1.5 ml tube. Tubes were centrifuged at 10,000×g for 15 min. The spore pellet was recovered and washed three times with ice-cold sterile water. Spore purity was assessed by phase contrast microscopy and spore yields in individual preparations were estimated by counting colony-forming units (CFU) of heat-treated (60° C., 20 min) aliquots on BHIS agar plates (Brain heart infusion supplemented with 0.1% L-cysteine and 5 mg ml⁻¹ yeast extract) supplemented with 0.1% sodium taurocholate (BHISS).

### ClosTron Mutagenesis

Insertional mutations in the bclA genes were made using the ClosTron system developed at the University of Nottingham (Heap et al., 2007, Heap et al., 2009, Heap et al., 2010). The Perutka algorithm (Perutka et al., 2004) available at www.clostron.com was used to design 45-bp retargeting sequences for each gene (Table 1). Derivatives of plasmid pMTL007C-E2 carrying these retargeting sequences were obtained from DNA2.0 (DNA20.com, Menlo Park, USA). Using the protocols provided by Heap et al (Heap et al., 2007, Heap et al., 2009, Heap et al., 2010) plasmids were first introduced into E. coli and then conjugated with C. difficile 6300 erm. For each mutant five erythromycin-resistant (ErmR) transconjugants were checked by PCR for ClosTron insertion. Genomic DNA was prepared as described (Antunes et al., 2011) and then three PCR reactions were performed (FIG. 15). First, PCR using the ErmRAM primers resulted in a 900 bp product confirming that the ErmR phenotype was due to splicing of the group I intron from the group II intron following integration. Second, primers targeting the gene left and right ends of the insertion site were used to check the site of insertion. If insertion occurred a PCR product 1800 bp greater than that obtained in the wild-type strain would be found. Third, PCR was made using primers flanking the gene and intron (EBS-universal) to confirm the insertion site where no product would be expected in the wild type strain.

Complementation of bclA Mutants

All three bclA mutants were complemented with wild-type copies of the respective genes using pRPF185 (Fagan & Fairweather, 2011). Briefly, a DNA fragment including the entire coding sequence of each gene and Shine-Dalgarno sequence was PCR amplified using KOD Hot Start polymerase (Merck) and primers listed in Table 5.

TABLE 5
Primers for construction of complementation vectors

| | | | Restriction |
|---|---|---|---|
| | Direction | Sequences¹ 5′-3′ | site |
| bclA1-SacI-F | forward | GATCGAGCTCTGATATAGACCCAAAATGGAG [SEQ ID | SacI |
| | | No: 25] | |
| bclA1-BamHI-R | reverse | GATCGGATCCAGTTTTTAAGATTATTTTAGACACG | BamHI |
| | | [SEQ ID No: 26] | |
| bclA2-BamHI-F | forward | GATCGGATCCCTTTTCATCATATAAACTATTGTATTC | BamHI |
| | | [SEQ ID No: 27] | |
| bclA2-SacI-R | reverse | GATCGAGCTCATTACTCTAACTTTAAAAAAGGAGG | SacI |
| | | [SEQ ID No: 28] | |
| bclA3-BamHI-F | forward | GATCGGATCCCACTTATATGGCATACTGTCT [SEQ ID | BamHI |
| | | No: 29] | |
| bclA3-SacI-R | reverse | GATCGAGCTCGCTTAAAAGCTCAAATATATCAGG | SacI |
| | | [SEQ ID No: 30] | |

The resulting fragments were cloned using Sad and BamHI sites into pRPF185 under the control of the inducible Pₜₑₜ promoter. Plasmids were transferred into the corresponding bclA mutant strains by conjugation. Gene expression was induced using anhydrous tetracycline (ATc) at 500 ng ml⁻¹. To confirm that the bclA mutants were due to a single insertional mutation we used in trans complementation analysis to demonstrate that the wild-type phenotype could be restored using two methods; i) immunofluorescence microscopy of spores to demonstrate surface expression of the BclA protein on spores of the complemented strain, and ii) restoration of wild type levels of germination (FIG. 16).

### Germination Assays

Spore germination was carried out in a 96-well plate (Greiner Bio-One) and germination of spores was measured by the percentage change in OD₆₀₀. HistoDenz-purified spores at an OD₆₀₀ of ˜0.8-1.0 (˜1×10⁸ ml⁻¹) were pelleted by centrifugation (10,000 g, 1 min) and suspended in 1 ml of BHIS supplemented with 0.1% sodium taurocholate (germinant) or 0.1% sodium chenodeoxycholate (inhibitor). The initial OD₆₀₀ was recorded and then measured at 1 minute intervals over 30 minutes using a microplate reader (Molecular Devices, Spectramax plus). % germination was determined as recorded OD600 at time interval/initial OD₆₀₀)×100. The experiment was performed three times. For preparations of sonicated spores ten cycles of sonication were used as described elsewhere (Permpoonpattana et al., 2011b).

### SATH (Spore Adhesion to Hydrocarbon) Assay

As described elsewhere (Huang et al., 2010) HistoDenz-purified spores were washed in 1M NaCl and then suspended in 0.1M NaCl for assay. 500 μl of spore suspension was added to 800 μl n-hexadecane (Sigma) and vortexed for 1 min. Samples were then incubated for 10 min at 37° C. with mild agitation, vortexed (30 s) and absorbance (OD₆₀₀ₙₘ) read. % hydrophobicity was determined from the absorbance of the original spore suspension (A₁) and the absorbance of the aqueous phase after incubation with hydrocarbon (A₂) using the equation: % H=[(A₁−A₂)/A₁].

### Recombinant Proteins and Antibody Production

E. coli pET28b expression vectors carrying the bclA1, bclA2 and bclA3 ORFs were used to express rBclA proteins. The segments of BclA used for expression were rBclA1 (Met-1 to Pro-393), rBclA2 (Met-1 to Gly-302) and rBclA3 (Thr-489 to Ala-645). High levels of expression were obtained upon IPTG induction and purification of proteins made by passage of the cell lysate through a HiTrap chelating HP column on a Pharmacia AKTA liquid chromatography system. Polyclonal antibodies were raised in Balb/c mice immunized by the intra-peritoneal route with 2 μg of purified recombinant proteins on days 1, 14 and 28. Antibodies were first purified using a Protein G HP Spin-Trap column (GE Healthcare).

### Transmission Electron Microscopy (TEM)

Spores were processed for ultra-microtomy according to standard procedures (Hong et al., 2009). Briefly, spore suspensions were diluted 10× in dH₂O and washed twice by centrifugation (10,000 g for 10 min) to eliminate residual debris. Spore pellets were fixed for 12 h at 4° C. in a mixture of 2.5% glutaraldehyde and 4% paraformaldehyde in 0.2 M cacodylate buffer (pH 7.4), then post-fixed for 1 h at RT with 1% osmium tetroxide in the same buffer. Sample pellets were dehydrated with ethanol and embedded in Epon-Araldite. Thin sections were stained successively with 5% uranyl acetate and 1% lead citrate. TEM observation was performed with a FEI CM120 operated at 120 kV.

### Immunofluorescence Microscopy

The procedure followed was as described in Duc et al (Duc et al., 2003) with minor modifications. Microscope coverslips were first treated with 0.01% poly-L-lysine overnight. Spores (1×10⁷ were added to the slide and dried for 1 h at RT. After three washes with PBS (pH 7.4) and blocking in PBS+2% BSA+0.05% Tween-20 for 1.5 h, the first antibody was added (1:1000). Spores were incubated for 30 min at RT followed by three washes with PBS+0.05% Tween-20 after which anti-mouse-TTFC sera (1:1000) was added and incubated for 30 min at RT. After six more washes the slide was viewed under a Nikon Eclipse Ti—S fluorescence microscope.

### Colonization Experiments

a) infection of mice using cefoperazone pre-treatment: the cefoperazone murine model was initially used since the erythromycin-resistance cassette used in ClosTron mutants may not confer the same level of resistance to clindamycin as seen in the parental strain, depending upon its chromosomal location although this was found in this work to be unfounded (N. Fairweather per. comm.). Groups (n=4) of C57BL/6 mice (6-8 week old; female, Charles River) were administered with five doses of cefoperazone (MP Biomedicals), LLC (100 mg/kg; by intra-gastric gavage) on day 1, 3, 5, 7 and 9 using a procedure previously described (Theriot et al., 2011). Animals were kept in IVCs (independently ventilated cages) under sterile conditions. On day 10, mice were orogastrically (o.g.) infected with C. difficile 1×10⁴ spores/mouse of the wild type 630Δerm strain or one of the three bclA mutants (one group/mutant). Fresh feces from individually infected mice were collected on day 1, 3, 5, and 7 post-challenge. Samples were reconstituted in PBS supplemented with protease inhibitor (Thermo Scientific) using a ratio of 1:5 (weight feces (g): volume (ml)). Total counts and spore counts of C. difficile were performed by plating serial dilutions on BHIS and BHISS respectively, media was supplemented with cefoxitin and cycloserine (Bioconnections, Knypersley, UK). Spore counts were determined after heat-treating (60° C., 30 min) samples, serial dilution and plating for CFU/ml.

b) infection of mice using clindamycin pre-treatment: on days 1 and 3 animals received a single dose of clindamycin (30 mg/kg) as described above for cefoperazone (a) and they were kept in IVCs under sterile conditions. On day 8, animals were o.g. infected with different doses (ranging from 10² to 10⁴ spores/mouse) of C. difficile strains R20291, 630Δerm or bclA1⁻mutant (n.b., the 630Δerm and bclA1⁻ mutant are sensitive to clindamycin). Spore counts in freshly voided feces were determined after ethanol treatment (100% ethanol, 20 min) by plating as described above (a).

c) analysis in mice of in vivo toxin levels and spore kinetics: groups of 9-10 mice were administered with clindamycin as described above (b) and housed in IVCs. On day 8, mice were o.g. infected with spores of C. difficile wild type 630Δerm and bclA1 mutant strains at the dose of 1×10⁵ spores/mouse. Caeca from infected mice were aseptically removed 24 or 36 hours post-challenge. Samples were processed as described above (a). For detection of levels of toxin A and toxin B in caecum, samples were centrifuged for 10 min (10,000 g; 4° C.) and supernatants sterilized using 0.2 μM filters. An ELISA assay was performed following the method described below (toxin detection, e).

d) hamster infections: Golden Syrian Hamsters (female, aged 10 months; ˜100 g; Charles River) housed in IVCs were dosed o.g. with clindamycin (30 mg/kg) and infected 5 days later with C. difficile spores of the wild type 630Δerm strain or bclA1⁻ mutant at doses of either 102, 10³ or 10⁴ spores/hamster. Hamsters were then monitored for signs of disease progression and, based on severity of symptoms, culled upon reaching the clinical end point. Cecum samples were examined for toxin B by ELISA as described below. Toxin cytotoxicity assays using HT29 cells was assessed as described previously (Permpoonpattana et al., 2011a). Spore counts in caeca was performed as described above (b). Statistical significance between groups was calculated using a student's t-test.

e) toxin detection: toxins were extracted using a protease inhibitor buffer as described previously (Permpoonpattana et al., 2011a) and detected by a capture ELISA method. ELISA plates (Greiner, high binding) were coated with rabbit polyclonal antibodies against toxin A or toxin B (Meridian Life Science; 1 μg/mL in PBS buffer). Nates were blocked with 2% BSA (1 h, 30° C.), 10 μg of samples and 2 μl of reference toxin A or toxin B (Ab Serotec) were added to plates and incubated at 30° C. for 3 h. Monoclonal antibodies against toxin A (1/500) and toxin B (1/500) were used for detection (1 h, 30° C.). HRP-conjugated anti-mouse IgG was added as secondary antibody (1 h, RT). Nates were developed with TMB (Sigma). The sensitivity of the assays for both toxin A and B is 7 ng/ml.

### Creation of Fusion Genes/Proteins

FIG. 27 shows schematic drawings of plasmids (pTS16 and pTS20) used to create fusion proteins using CotB as a carrier, and FIG. 28 shows schematic drawings of plasmids (pTS17, and pTS19) used to create fusion proteins using CotC as a carrier.

### Plasmids

| Plasmid name | Genotype | Antibiotic resistance |
|---|---|---|
| pTS16 | cotB-bclA1 in pDG364 | amp^{R} |
| pTS17 | cotC-bclA1 in pDG364 | amp^{R} |
| pTS19 | cotC-cotE in pDG364 | amp^{R} |
| pTS20 | cotB-bclA1-cotE in | amp^{R} |
| | pDG364 | |
| pTS21 | cotC-bclA1-cotE in | amp^{R} |
| | pDG364 | |
| pTS16 | cotB-bclA1 in pDG364 | amp^{R} |

### Primers

| | | | Position of |
|---|---|---|---|
| | | | annealing |
| Primer | | Restriction | within |
| name | Sequence | site | sequence |
| BclA-for | AGATCTatgagaaatattatac [SEQ ID No: 31] | BglII | +1/+16 |
| BclA-rev | GTCGACTTATTAGCTAGCaaaaaaagtaatcaac | SalI, NheI | +128/+144 |
| | [SEQ ID No: 32] | | |
| CotE-for | AGATCTGCTAGCccaactaatgtagatgc [SEQ | BglII, NheI | +1171/+1188 |
| | ID No: 33] | | |
| CotE-rev | GTCGACTTAttggtcataagttgatg [SEQ ID | SalI | +2033/+2050 |
| | No: 34] | | |

^{a}Lowercase and capital letters indicate nucleotides complementary to corresponding gene DNA of C. difficile and unpaired flanking sequences carrying a restriction site, respectively.
^{b}Underlined letters indicate stop codons which have been inserted.
^{c}Referred to bclA or cotE sequences, taking the first nucleotide of the initiation codon as +1.

### Strains Made

| Strain | Genotype | BclA1^{a} | CotE^{a} | Resistance |
|---|---|---|---|---|
| PY79 | wild type | − | − | — |
| LS1 | cotB-bclA1 | + | − | cm^{R} |
| LS2 | cotC-bclA1 | + | − | spc^{R} cm^{R} |
| LS3 | cotB-bclA1-cotE | + | + | cm^{R} |
| LS4 | cotC-cotE | − | + | spc^{R} cm^{R} |
| LS5 | cotC-bclA1-cotE | + | + | spc^{R} cm^{R} |

^{a}Information based on the Western blot analysis performed with specific anti-CotB and anti-CotC antibodies.
spc^{R} = spectinomycin resistance (100 μg/ml),
cm^{R} = chloramphenicol resistance (5 μg/ml). Spectinomycin resistance originates from the spc gene inserted into the cotC gene of PY79.

### Methods

Fragments of cotB and cotC DNA (promoter plus coding sequence) were obtained from pNS₄ [1] and pM10 [2] plasmids. PCR was used to amplify these sequences and cloned into the pDG364 plasmid [3, 4]. Next PCR was used to clone the BclA1 and CotE sequences from C. difficile and which were cloned into the pDG364 clones that carried the corresponding CotB or CotC N-terminal sequences. Cloning was achieved by using embedded restriction endonuclease sites in the primers.

For example, to construct LS1 which expresses CotB-BclA1. That is the N-terminal 48 aa of BclA1 is fused to the C-terminus of the CotB protein and displayed on the spore coat of PY79 spores. To achieve this, PCR primers were used to first amplify CotB from pNS₄ which were cloned into the plasmid pDG364 to create pDG364-CotB. Cloning was achieved by PCR evaluation. Next, the BclA1 gene was amplified using PCR from C. difficile such that the PCR product carried BglII and NheI ends. This then enabled cloning into precut pDG364-CotB using ligation of sticky ends. The recombinant plasmid pTS16 was then linearized using restriction enzymes that cut the pDG364 backbone and linearized DNA transformed into competent cells of B. subtilis strain PY79 with selection of Cm^{R} (5 μg/ml) as described elsewhere. Transformants were then purified by restreaking and spores of the strain made as described [5]. Proteins were extracted from the spore coats as described [6] and fractionated on SDS-PAGE gels and western blotted with antibodies to CotB (see FIGS. 29 and 30).

For clones LS1 and LS3 coat proteins were proved with anti-CotB antibodies which demonstrated a band shift for the chimeric protein.

For LS2, LS4 and LS5 anti-CotC antibodies were used. Note that for these 3 strains the linearized pDG364 plasmid is transferred into a cotC::spc mutant which carries Spc^{R} (spectinomycin resistance). Expression of CotC-chimeras has been shown to be enhanced in the absence of a wild type CotC protein whereas for CotB it is preferred to have a wild type copy of CotB present. This is in contrast to LS1 and LS3 where the linearized pDG364 plasmid is transformed into a PY79 wild type strain which carries no resistance gene.

### REFERENCES

[1] Isticato R, Cangiano G, Tran H T, Ciabattini A, Medaglini D, Oggioni M R, et al. Surface display of recombinant proteins on Bacillus subtilis spores. Journal of bacteriology. 2001; 183:6294-301.
[2] Mauriello E M, Duc le H, Isticato R, Cangiano G, Hong H A, De Felice M, et al. Display of heterologous antigens on the Bacillus subtilis spore coat using CotC as a fusion partner. Vaccine. 2004; 22:1177-87.
[3] Cutting S M, Azevedo V. Genetic mapping in Bacillus subtilis. In: Adolph. K W, editor. Microbial Gene Techniques. San Diego, Calif.: Academic Press, Inc.; 1995. p. 323-38.
[4] Guerout-Fleury A M, Frandsen N, Stragier P. Plasmids for ectopic integration in Bacillus subtilis. Gene. 1996; 180:57-61.
[5] Cutting S M, Vander-Horn P B. Genetic Analysis. In: Harwood C R, Cutting S M, editors. Molecular Biological Methods for Bacillus. Chichester, England: John Wiley & Sons Ltd.; 1990. p. 27-74.
[6] Nicholson W L, Setlow P. Sporulation, germination and outgrowth. In: Harwood. C R, Cutting. S M, editors. Molecular Biological Methods for Bacillus. Chichester, U K.: John Wiley & Sons Ltd.; 1990. p. 391-450.

### Further References

Antunes, A., I. Martin-Verstraete & B. Dupuy, (2011) CcpA-mediated repression of Clostridium difficile toxin gene expression. Mol Microbiol 79: 882-899.
Boydston, J. A., P. Chen, C. T. Steichen & C. L. Turnbough, Jr., (2005) Orientation within the exosporium and structural stability of the collagen-like glycoprotein BclA of Bacillus anthracis. J Bacteriol 187: 5310-5317.
Bozue, J., C. K. Cote, K. L. Moody & S. L. Welkos, (2007) Fully virulent Bacillus anthracis does not require the immunodominant protein BclA for pathogenesis. Infect Immun 75: 508-511.
Brahmbhatt, T. N., B. K. Janes, E. S. Stibitz, S. C. Darnell, P. Sanz, S. B. Rasmussen & A. D. O'Brien, (2007) Bacillus anthracis exosporium protein BclA affects spore germination, interaction with extracellular matrix proteins, and hydrophobicity. Infect Immun 75: 5233-5239.
Buckley, A. M., J. Spencer, D. Candlish, J. J. Irvine & G. R. Douce, (2011) Infection of hamsters with the U K Clostridium difficile ribotype 027 outbreak strain R20291. J Med Microbiol 60: 1174-1180.
Buckley, A. M., J. Spencer, L. M. Maclellan, D. Candlish, J. J. Irvine & G. R. Douce, (2013) Susceptibility of hamsters to Clostridium difficile isolates of differing Toxinotype. PLoS One 8: e64121.
Carr, K. A., S. R. Lybarger, E. C. Anderson, B. K. Janes & P. C. Hanna, (2010) The role of Bacillus anthracis germinant receptors in germination and virulence. Mol Microbiol 75: 365-375.
Dapa, T. & M. Unnikrishnan, (2013) Biofilm formation by Clostridium difficile. Gut microbes 4.
Daubenspeck, J. M., H. Zeng, P. Chen, S. Dong, C. T. Steichen, N. R. Krishna, D. G. Pritchard & C. L. Turnbough, Jr., (2004) Novel oligosaccharide side chains of the collagen-like region of BclA, the major glycoprotein of the Bacillus anthracis exosporium. The Journal of Biological Chemistry 279: 30945-30953.
Dawson, L. F., E. Valiente, A. Faulds-Pain, E. H. Donahue & B. W. Wren, (2012) Characterisation of Clostridium difficile biofilm formation, a role for SpooA. PLoS One 7: e50527.
Deakin, L. J., S. Clare, R. P. Fagan, L. F. Dawson, D. J. Pickard, M. R. West, B. W. Wren, N. F. Fairweather, G. Dougan & T. D. Lawley, (2012) The Clostridium difficile spooA Gene Is a Persistence and Transmission Factor. Infect Immun 80: 2704-2711.
Duc, L. H., H. A. Hong, N. Fairweather, E. Ricca & S. M. Cutting, (2003) Bacterial spores as vaccine vehicles. Infection and Immunity 71: 2810-2818.
Escobar-Cortes, K., J. Barra-Carrasco & D. Paredes-Sabja, (2013) Proteases and sonication specifically remove the exosporium layer of spores of Clostridium difficile strain 630. J Microbiol Methods 93: 25-31.
Fagan, R. P. & N. F. Fairweather, (2011) Clostridium difficile has two parallel and essential Sec secretion systems. J Biol Chem 286: 27483-27493.
Gerding, D. N., C. A. Muto & R. C. Owens, Jr., (2008) Measures to control and prevent Clostridium difficile infection. Clin Infect Dis 46 Suppl 1: S43-49.
Goulding, D., H. Thompson, J. Emerson, N. F. Fairweather, G. Dougan & G. R. Douce, (2009) Distinctive profiles of infection and pathology in hamsters infected with Clostridium difficile strains 630 and B1. Infect Immun 77: 5478-5485.
Heap, J. T., S. A. Kuehne, M. Ehsaan, S. T. Cartman, C. M. Cooksley, J. C. Scott & N. P. Minton, (2010) The ClosTron: Mutagenesis in Clostridium refined and streamlined. J Microbiol Methods 80: 49-55.
Heap, J. T., O. J. Pennington, S. T. Cartman, G. P. Carter & N. P. Minton, (2007) The ClosTron: a universal gene knock-out system for the genus Clostridium. J Microbiol Methods 70: 452-464.
Heap, J. T., O. J. Pennington, S. T. Cartman & N. P. Minton, (2009) A modular system for Clostridium shuttle plasmids. J Microbiol Methods 78: 79-85.
Henriques, A. O. & C. P. Moran, Jr., (2007) Structure, assembly, and function of the spore surface layers. Annu Rev Microbiol 61: 555-588.
Hong, H. A., R. Khaneja, N. M. Tam, A. Cazzato, S. Tan, M. Urdaci, A. Brisson, A. Gasbarrini, I. Barnes & S. M. Cutting, (2009) Bacillus subtilis isolated from the human gastrointestinal tract. Res Microbiol 160: 134-143.
Huang, J. M., H. A. Hong, H. Van Tong, T. H. Hoang, A. Brisson & S. M. Cutting, (2010) Mucosal delivery of antigens using adsorption to bacterial spores. Vaccine 28: 1021-1030.
Hussain, H. A., A. P. Roberts & P. Mullany, (2005) Generation of an erythromycin-sensitive derivative of Clostridium difficile strain 630 (630Derm) and demonstration that the conjugative transposon Tn916D E enters the genome of this strain at multiple sites. J Med Microbiol 54: 137-141.
Kailas, L., C. Terry, N. Abbott, R. Taylor, N. Mullin, S. B. Tzokov, S. J. Todd, B. A. Wallace, J. K. Hobbs, A. Moir & P. A. Bullough, (2011) Surface architecture of endospores of the Bacillus cereus/anthracis/thuringiensis family at the subnanometer scale. Proc Natl Acad Sci USA 25 108:16014-16019.
Kuehne, S. A., S. T. Cartman, J. T. Heap, M. L. Kelly, A. Cockayne & N. P. Minton, (2010) The role of toxin A and toxin B in Clostridium difficile infection. Nature 467: 711-713.
Lawley, T. D., S. Clare, A. W. Walker, D. Goulding, R. A. Stabler, N. Croucher, P. Mastroeni, P. Scott, C. Raisen, L. Mottram, N. F. Fairweather, B. W. Wren, J. Parkhill & G. Dougan, (2009a) Antibiotic treatment of Clostridium difficile carrier mice triggers a supershedder state, spore-mediated transmission, and severe disease in immunocompromised hosts. Infect Immun 77: 3661-3669.
Lawley, T. D., N. J. Croucher, L. Yu, S. Clare, M. Sebaihia, D. Goulding, D. J. Pickard, J. Parkhill, J. Choudhary & G. Dougan, (2009b) Proteomic and genomic characterization of highly infectious Clostridium difficile 630 spores. J Bacteriol 191: 5377-5386.
Nagaro, K. J., S. T. Phillips, A. K. Cheknis, S. P. Sambol, W. E. Zukowski, S. Johnson & D. N. Gerding, (2013) Nontoxigenic Clostridium difficile protects hamsters against challenge with historic and epidemic strains of toxigenic BI/NAP1/027 C. difficile. Antimicrob Agents Chemother 57: 5266-5270.
Oliva, C., C. L. Turnbough, Jr. & J. F. Kearney, (2009) CD14-Mac-1 interactions in Bacillus anthracis spore internalization by macrophages. Proc Natl Acad Sci USA 106: 13957-13962.
Ozanne, G., (1984) Estimation of endpoints in biological systems. Computers in biology and medicine 14: 377-384.
Paredes-Sabja, D., C. Bond, R. J. Carman, P. Setlow & M. R. Sarker, (2008) Germination of spores of Clostridium difficile strains, including isolates from a hospital outbreak of Clostridium difficile-associated disease (CDAD). Microbiology 154: 2241-2250.
Permpoonpattana, P., H. A. Hong, J. Phetcharaburanin, J. M. Huang, J. Cook, N. F. Fairweather & S. M. Cutting, (2011a) Immunization with Bacillus spores expressing toxin A peptide repeats protects against infection with Clostridium difficile strains producing toxins A and B. Infection and immunity 79: 2295-2302.
Permpoonpattana, P., J. Phetcharaburanin, A. Mikelsone, M. Dembek, S. Tan, M. C. Brisson, R. La Ragione, A. R. Brisson, N. Fairweather, H. A. Hong & S. M. Cutting, (2013) Functional Characterization of Clostridium difficile Spore Coat Proteins. J Bacteriol 195: 1492-1503.
Permpoonpattana, P., E. H. Tolls, R. Nadem, S. Tan, A. Brisson & S. M. Cutting, (2011b) Surface layers of Clostridium difficile endospores. J Bacteriol 193: 6461-6470.
Perutka, J., W. Wang, D. Goerlitz & A. M. Lambowitz, (2004) Use of computer-designed group II introns to disrupt Escherichia coli DExH/D-box protein and DNA helicase genes. J Mol Biol 336: 421-439.
Pizarro-Guajardo, M., V. Olguín-Araneda, J. Barra-Carrasco, C. Brito-Silva, M. R. Sarker & D. Paredes-Sabja., (2013) Characterization of the collagen-like exosporium protein, BclA1 of Clostridium difficile spores. Anaerobe In press.
Razaq, N., S. Sambol, K. Nagaro, W. Zukowski, A. Cheknis, S. Johnson & D. N. Gerding, (2007) Infection of hamsters with historical and epidemic B I types of Clostridium difficile. J Infect Dis 196: 1813-1819.
Redmond, C., L. W. Baillie, S. Hibbs, A. J. Moir & A. Moir, (2004) Identification of proteins in the exosporium of Bacillus anthracis. Microbiology 150: 355-363.
Rupnik, M., M. H. Wilcox & D. N. Gerding, (2009) Clostridium difficile infection: new developments in epidemiology and pathogenesis. Nat Rev Microbiol 7: 526-536.
Sambol, S. P., J. K. Tang, M. M. Merrigan, S. Johnson & D. N. Gerding, (2001) Infection of hamsters with epidemiologically important strains of Clostridium difficile. J Infect Dis 183: 1760-1766.
Sebaihia, M., B. W. Wren, P. Mullany, N. F. Fairweather, N. Minton, R. Stabler, N. R. Thomson, A. P. Roberts, A. M. Cerdeno-Tarraga, H. Wang, M. T. Holden, A. Wright, C. Churcher, M. A. Quail, S. Baker, N. Bason, K. Brooks, T. Chillingworth, A. Cronin, P. Davis, L. Dowd, A. Fraser, T. Feltwell, Z. Hance, S. Holroyd, K. Jagels, S. Moule, K. Mungall, C. Price, E. Rabbinowitsch, S. Sharp, M. Simmonds, K. Stevens, L. Unwin, S. Whithead, B. Dupuy, G. Dougan, B. Barrell & J. Parkhill, (2006) The multidrug-resistant human pathogen Clostridium difficile has a highly mobile, mosaic genome. Nat Genet 38: 779-786.
Songer, J. G. & M. A. Anderson, (2006) Clostridium difficile: an important pathogen of food animals. Anaerobe 12: 1-4.
Stabler, R. A., M. He, L. Dawson, M. Martin, E. Valiente, C. Corton, T. D. Lawley, M. Sebaihia, M. A. Quail, G. Rose, D. N. Gerding, M. Gibert, M. R. Popoff, J. Parkhill, G. Dougan & B. W. Wren, (2009) Comparative genome and phenotypic analysis of Clostridium difficile 027 strains provides insight into the evolution of a hypervirulent bacterium. Genome Biol 10: R102.
Steichen, C., P. Chen, J. F. Kearney & C. L. Turnbough, Jr., (2003) Identification of the immunodominant protein and other proteins of the Bacillus anthracis exosporium. J Bacteriol 185: 1903-1910.
Sylvestre, P., E. Couture-Tosi & M. Mock, (2002) A collagen-like surface glycoprotein is a structural component of the Bacillus anthracis exosporium. Mol Microbiol 45: 169-178.
Sylvestre, P., E. Couture-Tosi & M. Mock, (2003) Polymorphism in the collagen-like region of the Bacillus anthracis BclA protein leads to variation in exosporium filament length. J Bacteriol 185: 1555-1563.
Tan, L. & C. L. Turnbough, Jr., (2010) Sequence motifs and proteolytic cleavage of the collagen-like glycoprotein BclA required for its attachment to the exosporium of Bacillus anthracis. J Bacteriol 192: 1259-1268.
Theriot, C. M., C. C. Koumpouras, P. E. Carlson, Bergin, I I—, D. M. Aronoff & V. B. Young, (2011) Cefoperazone-treated mice as an experimental platform to assess differential virulence of Clostridium difficile strains. Gut microbes 2: 326-334.
Thompson, B. M., J. M. Binkley & G. C. Stewart, (2011a) Current physical and SDS extraction methods do not efficiently remove exosporium proteins from Bacillus anthracis spores. J Microbiol Methods 85: 143-148.
Thompson, B. M., H. Y. Hsieh, K. A. Spreng & G. C. Stewart, (2011b) The co-dependence of BxpB/ExsFA and BclA for proper incorporation into the exosporium of Bacillus anthraces. Mol Microbiol 79: 799-813.
Thompson, B. M. & G. C. Stewart, (2008) Targeting of the BclA and BclB proteins to the Bacillus anthracis spore surface. Mol Microbiol 70: 421-434.
Todd, S. J., A. J. Moir, M. J. Johnson & A. Moir, (2003) Genes of Bacillus cereus and Bacillus anthracis encoding proteins of the exosporium. J Bacteriol 185: 3373-3378.
Waller, L. N., M. J. Stump, K. F. Fox, W. M. Harley, A. Fox, G. C. Stewart & M. Shahgholi, (2005) Identification of a second collagen-like glycoprotein produced by Bacillus anthracis and demonstration of associated spore-specific sugars. J Bacteriol 187: 4592-4597.
Wust, J., N. M. Sullivan, U. Hardegger & T. D. Wilkins, (1982) Investigation of an outbreak of antibiotic-associated colitis by various typing methods. J Clin Microbiol 16: 1096-1101.

1. A vaccine comprising a C. difficile BclA polypeptide, or a fragment thereof, and an adjuvant.

2. A vaccine according to claim 1, wherein the BclA polypeptide comprises an amino acid sequence of any one of SEQ ID No:4-6, or a fragment thereof, or the polypeptide is encoded by a nucleic acid sequence of any one of SEQ ID No:1-3, or a fragment thereof.

3. A vaccine according to claim 1, wherein the BclA polypeptide comprises the amino acid sequence of SEQ ID No:4, or a fragment thereof, or the polypeptide is encoded by the nucleic acid sequence of SEQ ID No:1, or a fragment thereof.

4. A vaccine according to claim 1, wherein the vaccine comprises the N-terminal 50 amino acids of the C. difficile BclA polypeptide.

5. A vaccine according to claim 1, wherein the vaccine consists of the first 48 amino acids forming the N-terminus of the C. difficile BclA polypeptide.

6. A vaccine according to claim 1, wherein the BclA polypeptide comprises or consists of amino acid residues 1-48, as set out in SEQ ID No:4.

7. A vaccine according to claim 1, wherein the BclA1 polypeptide comprises or consists of the amino acid sequence of SEQ ID No:7, or is encoded by the nucleic acid sequence of SEQ ID No:8.

8. A vaccine according to claim 1, wherein the vaccine comprises toxin A or toxin B, or C. difficile CotE, or a fragment thereof, or spores expressing one or more of: toxin A, CotE, BClA1-CotE fusion, or a fragment thereof.

9. A vaccine according to claim 1, wherein the vaccine comprises spores expressing said polypeptide or said fragment.

10. A vaccine according to claim 1 wherein the polypeptide or fragment is expressed as a fusion protein on spores.

11. A method of treating, ameliorating or preventing an infection with Clostridium spp. or Bacillus spp., the method comprising: administering, to a subject in need of such treating, ameliorating, or preventing, the vaccine according to claim 1.

12. A method according to claim 11, wherein the method comprises treating or ameoliorating a subject with an infection of B. anthracis, B. cereus, C. difficile, C. perfringens, C. tetani, C. botulinum, C. acetobutylicum, C. cellulolyticum, C. novyi or C. thermocellum, or preventing such an infection in a subject.

13. The method of claim 11, wherein the subject has a C. difficile infection.

14. A Clostridium spp. or Bacillus spp. detection kit comprising detection means arranged, in use, to detect, in a sample, the presence of a C. difficile BclA polypeptide, or a fragment thereof, wherein detection of the polypeptide or fragment thereof signifies the presence of Clostridium spp. or Bacillus spp., said kit comprising a C. difficile BclA polypeptide or a fragment thereof which is attached to a detectable label or to a solid support.

15. The kit according to claim 14, wherein the detection means detects only the N-terminus of the C. difficile BclA polypeptide.

16. The kit according to claim 14, wherein the detection means detects only the first 300, 200, 150, 100 or 50 amino acids forming the N-terminus of the C. difficile BclA polypeptide.

17. The kit according to claim 14, wherein the detection means comprises a polyclonal or monoclonal antibody.

18. A method of detecting Clostridium spp. or Bacillus spp., the method comprising the steps of detecting, in a sample, the presence of a C. difficile BclA polypeptide, or a fragment thereof, wherein detection of the polypeptide, or fragment thereof signifies the presence of Clostridium spp. or Bacillus spp.

19. The method according to claim 18, wherein the sample comprises Bacillus spp., B. anthracis, B. cereus, Clostridium spp., C. difficile, C. perfringens, C. tetani, C. botulinum, C. acetobutylicum, C. cellulolyticum, C. novyi or C. thermocellum.

20. The method according to claim 18, wherein the detection means detects the N-terminus of the C. difficile BclA polypeptide.

21. The method according to claim 18, wherein the detection means detects the first 300, 200, 150, 100 or 50 amino acids forming the N-terminus of the C. difficile BclA polypeptide.


SEQUENCE LISTING (34 sequences)

SEQ ID NO: 1 - DNA, 2082 bp, Clostridium difficile
atgagaaatattatactttatttaaatgatgatacttttatatctaaaaaatatccagataaaaactttagtaatttagattattgcttaataggaagtaaatgttcaaatagttttgtaaaagaaaagttgattactttttttaaagtgagaataccagatatattaaaagacaaaagtatattaaaagcagagttatttattcatattgattcaaataagaatcatatttttaaagaaaaagtagatattgaaattaaaagaataagtgaatattataatttacgaactataacatggaatgatagagtgtctatggaaaatatcaggggatatttaccaattgggataagtgatacatccaactatatttgtttaaatattacgggaactataaaagcatgggcaatgaataaatatcctaattatgggttagctttatctttaaattacccttatcagattcttgaatttacatctagtagaggttgtaacaaaccgtatatacttgtaacatttgaagatagaattatagataattgttatcctaaatgtgagtgtcctccaattagaattacaggtccaatgggaccaagaggagcgacaggaagtacaggaccaatgggagtaacaggcccaaccggaagtacaggagcgacaggaagcataggaccaacaggcccaaccggaaatacaggagcaacaggaagtatagggccaacgggagtaacaggcccaaccggaagtacaggagcgacaggaagtataggaccaacaggagtaacaggtccgacaggaaatacgggagtgacaggaagtataggaccaacgggagcaacaggcccgacaggaaatacgggagtgacaggaagtataggaccaacaggagtaacaggcccaacaggaaatacaggagaaataggaccaacgggagcaacaggtccaacaggagtgacaggaagtataggaccaacaggagcaacaggaccaacaggagaaataggaccaacgggagcaacaggagcgacaggaagtataggaccaacaggagcaacaggtccaacaggagcgacaggagtgacaggagaaatagggccaacaggagaaataggaccaacgggagcaacaggcccaacaggagtgacaggaagtataggaccaacgggagcaacaggcccaacaggagcgacaggagaaataggaccaacaggagcaacaggcccaacaggagtgacaggaagtataggaccaacgggagcaacaggcccaacaggagcgacaggagaaataggaccaacgggagcaacaggcccaacaggagtaacaggagaaataggaccaacgggagcaacaggcccaacaggaaatacaggagtaacaggagaaataggaccaacgggagcaacgggtccgacaggaaatacaggagtgacaggagaaataggaccaacgggagcaacaggaccaacaggagtgacaggagaaatagggccaacaggaaatacaggagcgacaggaagtatagggccaacgggagtaacaggtccaacaggagcgacaggaagtataggaccaacgggagcaacaggagcgacaggagtaacaggaccaacaggtccaacaggagcaacaggcaattcctctcagccagttgctaacttcctcgtaaatgcaccatctccacaaacactaaataatggagatgctataacaggttggcaaacaataataggaaatagttcaagtataacagtagatacaaatggtacgtttacagtacaagaaaatggtgtgtattatatatcagtttcagtagcattacaaccaggttcatcaagtataaatcaatattctttcgctatcctattcccaattttaggaggaaaagatttggcagggcttactactgagccaggaggcggaggagtactttctggatattttgctggttttttatttggtgggactacttttacaataaataatttttcatctacaacagtagggatacgaaatgggcaatcagcaggaactgcggctactttgacgatatttagaatagctgatactgttatgacttaa

SEQ ID NO: 2 - DNA, 1677 bp, Clostridium difficile
atgagtgatatttcaggtccaagtttatatcaagatgtaggtccaacagggccaacaggtgctactggtccaacaggaccgacggggcctagaggtgcaactggagcgaccggagcaaatggaataacaggaccaacaggaaatacaggagcaactggagcgaatggaataacgggtccaacaggaaatatgggagcgactggaccaaatggaacaacaggttctacaggaccaacaggaaatacaggagcgactggagcgaatggaataacaggtccgacagggaatacaggagcaaccggagcaaatggaataacaggaccaacaggaaacaaaggagcaaccggagcaaatggaataacaggttctacaggaccaacaggaaatacaggagcgactggagcaaatggaataacaggtccaacagggaatacaggagcaacaggagcaacaggtccaaccggactaacaggagcaacaggagcaaccggagcaaatggaataacaggaccaacaggaaatacaggagcaaccggagcaaatggagtaacaggtgctacaggcccaacaggaaatacaggagcaacaggtccaacaggaagtataggagcgactggagcaacaggaacaactggggcaacaggcccaataggagcaacaggagcaacaggagcagatggagaggtaggtccaacaggagcagtaggagcaacaggtccagatggtttggtaggtccaacaggcccaacaggcccaaccggagcaaccggagcaaatggtttggtaggcccaacaggcccaaccggagcaaccggagcaaatggtttggtaggtccaacaggagcgacaggagcaacaggagtagctggggcaataggtccaacgggagcagtaggagcaacaggcccaacgggagcagatggagcagtaggtccaacaggagcgacaggagcaacaggggcaaatggagcaacaggcccaacgggagcagtaggagcaacgggagcgaatggagtagcaggtccaataggtccaacaggtccaacgggagaaaatggagtagcaggagcaacaggagcgacaggagcaacaggggcaaatggagcaacaggcccaacaggagcagtaggagcaacgggagcaaatggagtagcaggagcgataggaccaacaggcccaaccggagcaaatggagcgacaggagcaacaggggcgacaggagcaacaggagcaaatggagcaacaggtccaaccggagcgacaggagcaacaggagtgttagcagcaaacaatgcacaatttacagtgtcctcttcaagtttagtgaataatacattagtgacatttaattcatcatttataaatggaactaatataacttttccaacaagtagtactataaatcttgcagttggagggatatacaatgtatctttcggtatacgtgccacactttcacttgcaggatttatgtcaattactactaactttaatggagtaactcaaaataactttattgcaaaagcagtaaatacacttacttcatcagatgtaagtgtaagtttaagctttttagttgatgctagagcagcagctgttactttaagttttacatttggttcaggcacgacaggtacttctgcagctggatatgtatcagtttatagaatacaatag

SEQ ID NO: 3 - DNA, 1986 bp, Clostridium difficile
gtgcttttaataatgagtagaaataaatattttggaccatttgatgataatgattacaacaatggctatgataaatatgatgattgtaacaatggtcgtgatgattataatagctgtgattgccatcattgctgtccaccatcatgtgtaggtccaacaggcccaatgggtccaagaggtagaaccggcccaacaggtccaacgggtccaacaggtccaggagtaggggcaacaggcccaacaggaccaaccggtccgactggtccaacaggaaatacagggaatacaggagcaacaggattaagaggtccaacaggagcaacaggggcaacaggcccaacaggagcgacaggagctatagggtttggagtaacaggtccaacaggcccaacaggagcgacaggagcaacaggagcagatggagtaacaggtccaacaggtccaacgggagcaacaggagcagatggaataacaggtccaacaggagcaacaggggcaacaggatttggagtaacaggtccaacaggcccaacaggagcaacaggagtaggagtaacaggagcaacaggattaataggtccaacaggagcgacaggaacacctggagcaacaggtccaacaggggcaataggagcaacaggaataggaataacaggtccaacaggagcaacaggagcaacaggggcagatggagcaacaggagtaacaggcccaacaggcccaacaggggcaacaggagcagatggagtaacaggcccaacaggagcaacaggagcaacaggaataggaataacaggcccaacaggtccaacaggagcaacaggaatagggataacaggggcaacaggattaataggtccaacaggggcaacaggaacacctggagcaacaggtccaacaggagcaacaggcccaacaggagtaggagtaacaggagcaacaggagcaacaggagcaacaggagcagacggagcaacaggagtaacaggtccaacaggagcaacaggggcaacaggagcaaatggattagtaggcccaacaggagccacaggagcagcaggaacacctggagcaacaggtccaacaggagcaacaggcccaacaggagtaggaataacaggagcaacaggggcaacaggagcgacaggtccaacaggagcagatggagcaacaggtccaacaggagcaacaggaaatacaggagcagatggagtagcaggtccaacaggagcaacaggaaatacaggagcagatggagcaacaggtccaacaggagcaacaggggcaacaggagcagatggagcaacaggtccaacaggagcaacaggagcaacaggagtggcaggagcaacaggagcaacaggtccaacaggagcaacaggagcagatggagcaacaggtccaacaggagcaacaggagcaacaggggcagatggagcaacaggtccaacaggagcaacaggggcaacaggagttacaggagcaacaggcccaacaggcccaacaggagcaacaggagcaacaggagcaacaggtgctagtgcaataataccttttgcatcaggtataccactatcacttacaactatagctggaggattagtaggtacacctggatttgttggatttggtagttcggctccaggattaagtatagttggtggagtaatagaccttacaaacgcagcaggaacattgactaactttgcattttcaatgccaagagatggaacaataacatctatttcagcatacttcagtacaacagcagcactttcacttgttggttcaacaattacaattacagcaacactttaccaatctactgcaccaaataactcatttacagctgtaccaggagcgacagttacactagctccaccacttacaggtatattatcagttggttcaatttctagtggaattgtaacaggattaaatatagcagcaacagcacaaactccagacagacagtatgccatataa

SEQ ID NO: 4 - protein, 693 aa, Clostridium difficile
MRNIILYLNDDTFISKKYPDKNFSNLDYCLIGSKCSNSFVKEKLITFFKVRIPDILKDKSILKAELFIHIDSNKNHIFKEKVDIEIKRISEYYNLRTITWNDRVSMENIRGYLPIGISDTSNYICLNITGTIKAWAMNKYPNYGLALSLNYPYQILEFTSSRGCNKPYILVTFEDRIIDNCYPKCECPPIRITGPMGPRGATGSTGPMGVTGPTGSTGATGSIGPTGPTGNTGATGSIGPTGVTGPTGSTGATGSIGPTGVTGPTGNTGVTGSIGPTGATGPTGNTGVTGSIGPTGVTGPTGNTGEIGPTGATGPTGVTGSIGPTGATGPTGEIGPTGATGATGSIGPTGATGPTGATGVTGEIGPTGEIGPTGATGPTGVTGSIGPTGATGPTGATGEIGPTGATGPTGVTGSIGPTGATGPTGATGEIGPTGATGPTGVTGEIGPTGATGPTGNTGVTGEIGPTGATGPTGNTGVTGEIGPTGATGPTGVTGEIGPTGNTGATGSIGPTGVTGPTGATGSIGPTGATGATGVTGPTGPTGATGNSSQPVANFLVNAPSPQTLNNGDAITGWQTIIGNSSSITVDTNGTFTVQENGVYYISVSVALQPGSSSINQYSFAILFPILGGKDLAGLTTEPGGGGVLSGYFAGFLFGGTTFTINNFSSTTVGIRNGQSAGTAATLTIFRIADTVMT

SEQ ID NO: 5 - protein, 558 aa, Clostridium difficile
MSDISGPSLYQDVGPTGPTGATGPTGPTGPRGATGATGANGITGPTGNTGATGANGITGPTGNMGATGPNGTTGSTGPTGNTGATGANGITGPTGNTGATGANGITGPTGNKGATGANGITGSTGPTGNTGATGANGITGPTGNTGATGATGPTGLTGATGATGANGITGPTGNTGATGANGVTGATGPTGNTGATGPTGSIGATGATGTTGATGPIGATGATGADGEVGPTGAVGATGPDGLVGPTGPTGPTGATGANGLVGPTGPTGATGANGLVGPTGATGATGVAGAIGPTGAVGATGPTGADGAVGPTGATGATGANGATGPTGAVGATGANGVAGPIGPTGPTGENGVAGATGATGATGANGATGPTGAVGATGANGVAGAIGPTGPTGANGATGATGATGATGANGATGPTGATGATGVLAANNAQFTVSSSSLVNNTLVTFNSSFINGTNITFPTSSTINLAVGGIYNVSFGIRATLSLAGFMSITTNFNGVTQNNFIAKAVNTLTSSDVSVSLSFLVDARAAAVTLSFTFGSGTTGTSAAGYVSVYRIQ

SEQ ID NO: 6 - protein, 661 aa, Clostridium difficile
MLLIMSRNKYFGPFDDNDYNNGYDKYDDCNNGRDDYNSCDCHHCCPPSCVGPTGPMGPRGRTGPTGPTGPTGPGVGATGPTGPTGPTGPTGNTGNTGATGLRGPTGATGATGPTGATGAIGFGVTGPTGPTGATGATGADGVTGPTGPTGATGADGITGPTGATGATGFGVTGPTGPTGATGVGVTGATGLIGPTGATGTPGATGPTGAIGATGIGITGPTGATGATGADGATGVTGPTGPTGATGADGVTGPTGATGATGIGITGPTGPTGATGIGITGATGLIGPTGATGTPGATGPTGATGPTGVGVTGATGATGATGADGATGVTGPTGATGATGANGLVGPTGATGAAGTPGATGPTGATGPTGVGITGATGATGATGPTGADGATGPTGATGNTGADGVAGPTGATGNTGADGATGPTGATGATGADGATGPTGATGATGVAGATGATGPTGATGADGATGPTGATGATGADGATGPTGATGATGVTGATGPTGPTGATGATGATGASAIIPFASGIPLSLTTIAGGLVGTPGFVGFGSSAPGLSIVGGVIDLTNAAGTLTNFAFSMPRDGTITSISAYFSTTAALSLVGSTITITATLYQSTAPNNSFTAVPGATVTLAPPLTGILSVGSISSGIVTGLNIAATAQTPDRQYAI

SEQ ID NO: 7 - protein, 48 aa, Clostridium difficile
MRKIILYLNDDTFISKKYPDKNFSNLDYCLIGSKCSNSFVKEKLITFF

SEQ ID NO: 8 - DNA, 147 bp, Clostridium difficile
atgagaaatattatactttatttaaatgatgatacttttatatctaaaaaatatccagataaaaactttagtaatttagattattgcttaataggaagtaaatgttcaaatagttttgtaaaagaaaagttgattacttttttttaa

SEQ ID NO: 9 - DNA, 1140 bp, Bacillus subtilis
atgagcaagaggagaatgaaatatcattcaaataatgaaatatcgtattataactttttgcactcaatgaaagataaaattgttactgtatatcgtggaggtccggaatctaaaaaaggaaaattaacagctgtaaaatcagattatatagctttacaagctgaaaaaaaaataatttattatcagttggagcatgtgaaaagtattactgaggataccaataatagcaccacaacaattgagactgaggaaatgctcgatgctgatgattttcatagcttaatcggacatttaataaaccaatcagttcaatttaaccaagggggtccggaatctaaaaaaggaagattggtctggctgggagatgattacgctgcgttaaacacaaatgaggatggggtagtgtattttaatatccatcacatcaaaagtataagtaaacacgagcctgatttgaaaatagaagagcagacgccagttggagttttggaagctgatgatttaagcgaggtttttaagagtctgactcataaatgggtttcaattaatcgtggaggtccggaagccattgagggtatccttgtagataatgccgacggccattatactatagtgaaaaatcaagaggtgcttcgcatctatccttttcacataaaaagcatcagcttaggtccaaaagggtcgtacaaaaaagaggatcaaaaaaatgaacaaaaccaggaagacaataatgataaggacagcaattcgttcatttcttcaaaatcatatagctcatcaaaatcatctaaacgatcactaaaatcttcagatgatcaatcatccaaatctggtcgttcgtcacgttcaaaaagttcttcaaaatcatctaaacgatcactaaaatcttcggattatcaatcatccaaatctggccgttcgtcacgttcaaaaagttcttcaaaatcatctaaacgatcattaaaatcttcagattatcaatcatcaaaatcatctaaacgatcaccaagatcttcagattatcaatcatcaagatcaccaggctattcaagttcaataaaaagttcaggaaaacaaaaggaagattatagctatgaaacgattgtcagaacgatagactatcactggaaacgtaaattt

SEQ ID NO: 10 - DNA, 516 bp, Bacillus subtilis
atgaaaaatcggctctttattttgatttgtttttgtgtcatctgtctttttctatcatttggacagcccttttttccttctatgattttaactgtccaagccgcaaaatctactcgccgtataataaagcgtagtaaaaataaaggaggagtatatagggttattacaaaaaatacaaagaagagtattatacggtcaaaaaaacgtattataagaagtattacgaatatgataaaaaagattatgactgtgattacgacaaaaaatatgatgactatgataaaaaatattatgatcacgataaaaaagactatgattatgttgtagagtataaaaagcataaaaaacactactaaacgccattaacatctcctcgtttttactttcccccggctattgccgggtcttttttgtttgtgcactatatgtatatttctgaagcttccctttctatgaaaaccttggtgactgaatctgaaaaagaatagtgaatatttagtacatagtttagacaaa

SEQ ID NO: 11 - DNA, 2138 bp, Clostridium difficile
gtgatttacatgccaaatttgccaagtttagggtcaaaggctcctgattttaaagccaatacaacaaatggtcctattagactctctgactataagggtaattggattgttttattttcacatcctggtgattttacaccagtttgtactacagaatttttatgttttgctaaatattatgacgaatttaaaaaaagaaatacagaactaattggtctaagtgttgatagtaacagttcacatttagcttggatgtataatatttctttacttacaggtgtagaaattccatttcctattatagaagatagagatatgagaattgccaagttatacggcatgatatcaaaaccaatgagtgatacatcaactgttcgctctgtatttattatagataataatcaaattctaagaacgattctttattatccactaactacaggaagaaatattccagaaatacttagaatagtagatgcacttcagactagtgatagagataatatagttactcctgcaaactggtttcctggaatgccagtgattttaccttatcctaaaaactataaggaattaaaaaatagagttaacagttgtaataagaaatattcatgtatggactggtacttatgttttgtaccagataattataatgatgaagaagtgagcaagaaaattgataatattgtagctggaaaaaagaacatactaaaaacattgaaaatgaatgtaactgtgaacatgaacatcatgactacctgaacaaagctcttgattgtaaacaagaacacaagactgatattaaagatgattgcaatcatgagaaaaaacatactaaaaatactaacaaagttcacaactccaaacaagataagtttaaagataagtcttgtgatgaaatgaattttaactatgacaaagatgaatcttgcgacaaaataaattctagctataacaaagaagatagtagttatgaagatttctataaacataattataaaaactacgattatactagcgaaaaaaatactaaaaaaatagctatgaaaactttaaaagattcaaaaaaattagttagaccacaaataacagacccatacaatccaatagttgaaaatgcaaactgtccagatataaatccaattgtagcagaatatgttcttggaaatccaactaatgtagatgctcaactattagatgcagttatatttgcttttgctgagatagaccagtctggaaatttgtttattccttatcctagatttttaaaccaattacttgctcttaaaggtgaaaaacctagcttaaaagtaattgtagctattggaggttggggagctgaaggtttctctgatgcagcattaacacctacatctagatataattttgcaagacaggtcaatcagatgataaatgaatatgctttagatggaatagatatagactgggaatatcctggaagtagtgcatctggaataacatcaagacctcaagatagagaaaactttacactcttactaactgccataagagatgttataggggatgataaatggcttagtgtagctggaacaggagatagaggatatataaattcaagtgctgaaatagataaaatagctcctataatagattattttaatcttatgagttatgattttacagcaggtgaaacaggcccaaatggtagaaaacatcaagcaaatctttttgattcagacttatctttgccaggatatagtgttgatgcaatggtgagaaatcttgagaatgctggaatgccttctgaaaaaatccttctcggtataccattttatggaagattaggtgctactataacaagaacttatgatgagcttagaagggattatataaataaaaatggatatgaatatagatttgataatactgctcaagttccgtatttagttaaggatggagattttgcaatgtcatatgatgatgctttatcaatattcttaaaaactcaatatgttcttagaaattgtctaggtggtgtattctcatggacatcaacttatgaccaagcaaatatactggctagaaccatgtctattggtataaatgaccctgaagtattaaaagaagaacttgaaggtatttatgggcaattctaa

SEQ ID NO: 12 - protein, 712 aa, Clostridium difficile
MIYMPNLPSLGSKAPDFKANTTNGPIRLSDYKGNWIVLFSHPGDFTPVCTTEFLCFAKYYDEFKKRNTELIGLSVDSNSSHLAWMYNISLLTGVEIPFPIIEDRDMRIAKLYGMISKPMSDTSTVRSVFIIDNNQILRTILYYPLTTGRNIPEILRIVDALQTSDRDNIVTPANWFPGMPVILPYPKNYKELKNRVNSCNKKYSCMDWYLCFVPDNYNDEEVSKKIDNTCSWKKEHTKNIENECNCEHEHHDYLNKALDCKQEHKTDIKDDCNHEKKHTKNTNKVHNSKQDKFKDKSCDEMNFNYDKDESCDKINSSYNKEDSSYEDFYKHNYKNYDYTSEKNTKKIAMKTLKDSKKLVRPQITDPYNPIVENANCPDINPIVAEYVLGNPTNVDAQLLDAVIFAFAEIDQSGNLFIPYPRFLNQLLALKGEKPSLKVIVAIGGWGAEGFSDAALTPTSRYNFARQVNQMINEYALDGIDIDWEYPGSSASGITSRPQDRENFTLLLTAIRDVIGDDKWLSVAGTGDRGYINSSAEIDKIAPIIDYFNLMSYDFTAGETGPNGRKHQANLFDSDLSLPGYSVDAMVRNLENAGMPSEKILLGIPFYGRLGATITRTYDELRRDYINKNGYEYRFDNTAQVPYLVKDGDFAMSYDDALSIFLKTQYVLRNCLGGVFSWTSTYDQANILARTMSIGINDPEVLKEELEGIYGQF

SEQ ID NO: 13 - DNA, 1169 bp, Unknown
  A construct comprising a nucleic acid sequence from C. difficile and B. subtilis
atgagcaagaggagaatgaaatatcattcaaataatgaaatatcgtattataactttttgcactcaatgaaagataaaattgttactgtatatcgtggaggtccggaatctaaaaaaggaaaattaacagctgtaaaatcagattatatagctttacaagctgaaaaaaaaataatttattatcagttggagcatgtgaaaagtattactgaggataccaataatagcaccacaacaattgagactgaggaaatgctcgatgctgatgattttcatagcttaatcggacatttaataaaccaatcagttcaatttaaccaagggggtccggaatctaaaaaaggaagattggtctggctgggagatgattacgctgcgttaaacacaaatgaggatggggtagtgtattttaatatccatcacatcaaaagtataagtaaacacgagcctgatttgaaaatagaagagcagacgccagttggagttttggaagctgatgatttaagcgaggtttttaagagtctgactcataaatgggtttcaattaatcgtggaggtccggaagccattgagggtatccttgtagataatgccgacggccattatactatagtgaaaaatcaagaggtgcttcgcatctatccttttcacataaaaagcatcagcttaggtccaaaagggtcgtacaaaaaagaggatcaaaaaaatgaacaaaaccagaagacaataatgataaggacagcaattcgttcatttcttcaaaatcatatagctcatcaaaatcatctaaacgatcactaaaatcttcagatgatcaatcatccaaatctggtcgttcgtcacgttcaaaaagttcttcaaaatcatctaaacgatcactaaaatcttcggattatcaatcatccaaatctggccgttcgtcacgttcaaaaagttcttcaaaatcatctaaacgatcattaaaatcttcagattatcaatcatcaaaatcatctaaacgatcaccaagatctatgagaaatattatactttatttaaatgatgatacttttatatctaaaaaatatccagataaaaactttagtaatttagattattgcttaataggaagtaaatgttcaaatagttttgtaaaagaaaagttgattactttttttgctagctaataa

SEQ ID NO: 14 - protein, 388 aa, Unknown
  A construct comprising a nucleic acid sequence from C. difficile and B. subtilis
MSKRRMKYHSNNEISYYNFLHSMKDKIVTVYRGGPESKKGKLTAVKSDYIALQAEKKIIYYQLEHVKSITEDTNNSTTTIETEEMLDADDFHSLIGHLINQSVQFNQGGPESKKGRLVWLGDDYAALNTNEDGVVYFNIHHIKSISKHEPDLKIEEQTPVGVLEADDLSEVFKSLTHKWVSINRGGPEAIEGILVDNADGHYTIVKNQEVLRIYPFHIKSISLGPKGSYKKEDQKNEQNQEDNNDKDSNSFISSKSYSSSKSSKRSLKSSDDQSSKSGRSSRSKSSSKSSKRSLKSSDYQSSKSGRSSRSKSSSKSSKRSLKSSDYQSSKSSKRSPRSMRNIILYLNDDTFISKKYPDKNFSNLDYCLIGSKCSNSFVKEKLITFFAS

SEQ ID NO: 15 - DNA, 2046 bp, Unknown
  A construct comprising a nucleic acid sequence from C. difficile and B. subtilis
atgagcaagaggagaatgaaatatcattcaaataatgaaatatcgtattataactttttgcactcaatgaaagataaaattgttactgtatatcgtggaggtccggaatctaaaaaaggaaaattaacagctgtaaaatcagattatatagctttacaagctgaaaaaaaaataatttattatcagttggagcatgtgaaaagtattactgaggataccaataatagcaccacaacaattgagactgaggaaatgctcgatgctgatgattttcatagcttaatcggacatttaataaaccaatcagttcaatttaaccaagggggtccggaatctaaaaaaggaagattggtctggctgggagatgattacgctgcgttaaacacaaatgaggatggggtagtgtattttaatatccatcacatcaaaagtataagtaaacacgagcctgatttgaaaatagaagagcagacgccagttggagttttggaagctgatgatttaagcgaggtttttaagagtctgactcataaatgggtttcaattaatcgtggaggtccggaagccattgagggtatccttgtagataatgccgacggccattatactatagtgaaaaatcaagaggtgcttcgcatctatccttttcacataaaaagcatcagcttaggtccaaaagggtcgtacaaaaaagaggatcaaaaaaatgaacaaaaccaggaagacaataatgataaggacagcaattcgttcatttcttcaaaatcatatagctcatcaaaatcatctaaacgatcactaaaatcttcagatgatcaatcatccaaatctggtcgttcgtcacgttcaaaaagttcttcaaaatcatctaaacgatcactaaaatcttcggattatcaatcatccaaatctggccgttcgtcacgttcaaaaagttcttcaaaatcatctaaacgatcattaaaatcttcagattatcaatcatcaaaatcatctaaacgatcaccaagatctatgagaaatattatactttatttaaatgatgatacttttatatctaaaaaatatccagataaaaactttagtaatttagattattgcttaataggaagtaaatgttcaaatagttttgtaaaagaaaagttgattactttttttgctagcccaactaatgtagatgctcaactattagatgcagttatatttgcttttgctgagatagaccagtctggaaatttgtttattccttatcctagatttttaaaccaattacttgctcttaaaggtgaaaaacctagcttaaaagtaattgtagctattggaggttggggagctgaaggtttctctgatgcagcattaacacctacatctagatataattttgcaagacaggtcaatcagatgataaatgaatatgctttagatggaatagatatagactgggaatatcctggaagtagtgcatctggaataacatcaagacctcaagatagagaaaactttacactcttactaactgccataagagatgttataggggatgataaatggcttagtgtagctggaacaggagatagaggatatataaattcaagtgctgaaatagataaaatagctcctataatagattattttaatcttatgagttatgattttacagcaggtgaaacaggcccaaatggtagaaaacatcaagcaaatctttttgattcagacttatctttgccaggatatagtgttgatgcaatggtgagaaatcttgagaatgctggaatgccttctgaaaaaatccttctcggtataccattttatggaagattaggtgctactataacaagaacttatgatgagcttagaagggattatataaataaaaatggatatgaatatagatttgataatactgctcaagttccgtatttagttaaggatggagattttgcaatgtcatatgatgatgctttatcaatattcttaaaaactcaatatgttcttagaaattgtctaggtggtgtattctcatggacatcaacttatgaccaataa

SEQ ID NO: 16 - protein, 681 aa, Unknown
  A construct comprising a nucleic acid sequence from C. difficile and B. subtilis
MSKRRMKYHSNNEISYYNFLHSMKDKIVTVYRGGPESKKGKLTAVKSDYIALQAEKKIIYYQLEHVKSITEDTNNSTTTIETEEMLDADDFHSLIGHLINQSVQFNQGGPESKKGRLVWLGDDYAALNTNEDGVVYFNIHHIKSISKHEPDLKIEEQTPVGVLEADDLSEVFKSLTHKWVSINRGGPEAIEGILVDNADGHYTIVKNQEVLRIYPFHIKSISLGPKGSYKKEDQKNEQNQEDNNDKDSNSFISSKSYSSSKSSKRSLKSSDDQSSKSGRSSRSKSSSKSSKRSLKSSDYQSSKSGRSSRSKSSSKSSKRSLKSSDYQSSKSSKRSPRSMRNIILYLNDDTFISKKYPDKNFSNLDYCLIGSKCSNSFVKEKLITFFASPTNVDAQLLDAVIFAFAEIDQSGNLFIPYPRFLNQLLALKGEKPSLKVIVAIGGWGAEGFSDAALTPTSRYNFARQVNQMINEYALDGIDIDWEYPGSSASGITSRPQDRENFTLLLTAIRDVIGDDKWLSVAGTGDRGYINSSAEIDKIAPIIDYFNLMSYDFTAGETGPNGRKHQANLFDSDLSLPGYSVDAMVRNLENAGMPSEKILLGIPFYGRLGATITRTYDELRRDYINKNGYEYRFDNTAQVPYLVKDGDFAMSYDDALSIFLKTQYVLRNCLGGVFSWTSTYDQ

SEQ ID NO: 17 - DNA, 516 bp, Unknown
  A construct comprising a nucleic acid sequence from C. difficile and B. subtilis
atgaaaaatcggctctttattttgatttgtttttgtgtcatctgtctttttctatcatttggacagcccttttttccttctatgattttaactgtccaagccgcaaaatctactcgccgtataataaagcgtagtaaaaataaaggaggagtatatatgggttattacaaaaaatacaaagaagagtattatacggtcaaaaaaacgtattataagaagtattacgaatatgataaaaaagattatgactgtgattacgacaaaaaatatgatgactatgataaaaaatattatgatcacgataaaaaagactatgattatgttgtagagtataaaaagcataaaaaacactacagatctatgagaaatattatactttatttaaatgatgatacttttatatctaaaaaatatccagataaaaactttagtaatttagattattgcttaataggaagtaaatgttcaaatagttttgtaaaagaaaagttgattactttttttgctagctaataa

SEQ ID NO: 18 - protein, 170 aa, Unknown
  A construct comprising a nucleic acid sequence from C. difficile and B. subtilis
MKNRLFILICFCVICLFLSFGQPFFPSMILTVQAAKSTRRIIKRSKNKGGVYMGYYKKYKEEYYTVKKTYYKKYYEYDKKDYDCDYDKKYDDYDKKYYDHDKKDYDYVVEYKKHKKHYRSMRNIILYLNDDTFISKKYPDKNFSNLDYCLIGSKCSNSFVKEKLITFFAS

SEQ ID NO: 19 - protein, 388 aa, Bacillus anthracis
MSNNNYSNGLNPDESLSASAFDPNLVGPTLPPIPPFTLPTGPTGPTGPTGPTGPTGPTGPTGGTGPTGPTGDTGTTGPTGPTGPTGPTGPTGDTGTTGPTGPTGPTGPTGPTGPTGPTGPTGPTGPTGPTGPTGPTGPTGPTGPTGDTGTTGPTGPTGPTGPTGPTGPTGPTGPTGPTGPTGPTGPTGPTGDTGTTGPTGPTGPTGPTGPTGDTGTTGPTGPTGPTGPTGPTGPTGPTGATGLTGPTGPTGPSGLGLPAGLYAFNSGGISLDLGINDPVPFNTVGSQFGTAISQLDADTFVISETGFYKITVIANTATASVLGGLTIQVNGVPVPGTGSSLISLGAPIVIQAITQITTTPSLVEVIVTGLGLSLALGTSASIIIEKVA

SEQ ID NO: 20 - protein, 284 aa, Bacillus cereus
MSNHNYSDGLNPDESLSASAFDPNLVGPTLPPIPPFTLPTGPTGPTGPTGPTGPTVPTGPTGATGPTGPTGPTGDTGATGPTGPTGDTGATGPTGPTGDTGATGLTGPTGDTGATGLTGPTGDTGATGLTGPTGPTGATGLTGPTGPSGLGLPAGLYAFNSGGISLDLGINDPLPFNTVGSQFGTAISQLDADTFVISETGFYKITVVANTATVSALGGLTIQVNGVPVPGTGSSLISLGAPIVIQAITQITTTPSLVEVIVTGLGLSLALGTSASIIIIEKIA

SEQ ID NO: 21 - protein, 48 aa, Clostridium difficile; identical to SEQ ID NO: 7

SEQ ID NO: 22 - DNA, 45 bp, Clostridium difficile
  Intron Positioned between nucleotides 30 and 31
actcctgtcgctcctgttggacctgttgctcctgttggtcctata

SEQ ID NO: 23 - DNA, 45 bp, Clostridium difficile
  Intron Positioned between nucleotides 30 and 31
gctccatttgctcctgttgctcctgtcgcccctgttgctcctgtc

SEQ ID NO: 24 - DNA, 45 bp, Clostridium difficile
  Intron Positioned between nucleotides 29 and 30
gtcgtgatgattataatagctgtgattgccatcattgctgtccac

SEQ ID NO: 25 - DNA, 31 bp, Unknown
  Primer
gatcgagctctgatatagacccaaaatggag

SEQ ID NO: 26 - DNA, 35 bp, Unknown
  Primer
gatcggatccagtttttaagattattttagacacg

SEQ ID NO: 27 - DNA, 37 bp, Unknown
  Primer
gatcggatcccttttcatcatataaactattgtattc

SEQ ID NO: 28 - DNA, 35 bp, Unknown
  Primer
gatcgagctcattactctaactttaaaaaaggagg

SEQ ID NO: 29 - DNA, 31 bp, Unknown
  Primer
gatcggatcccacttatatggcatactgtct

SEQ ID NO: 30 - DNA, 34 bp, Unknown
  Primer
gatcgagctcgcttaaaagctcaaatatatcagg

SEQ ID NO: 31 - DNA, 22 bp, Unknown
  Primer
agatctatgagaaatattatac

SEQ ID NO: 32 - DNA, 34 bp, Unknown
  Primer
gtcgacttattagctagcaaaaaaagtaatcaac

SEQ ID NO: 33 - DNA, 29 bp, Unknown
  Primer
agatctgctagcccaactaatgtagatgc

SEQ ID NO: 34 - DNA, 26 bp, Unknown
  Primer
gtcgacttattggtcataagttgatg